SEQUENCE LISTING (20 sequences)

SEQ ID NO: 1 - DNA, 21 bp, Artificial sequence
  synthetic sequence
tctgctctgcactcgtaatac

SEQ ID NO: 2 - DNA, 160 bp, Artificial sequence
  synthetic sequence
cgtacgtacgtacgtcgacggatgacagctcgcatctacgagctcgagatgacacagagtatcgcatctacgacacagtctctcgcgagctagagatgagtgatcgagctctgctcggcgcgctatagtgagtcgtattacgagtgcagagcagactcac

SEQ ID NO: 3 - DNA, 160 bp, Artificial sequence
  synthetic sequence
cgtacgtacgtacgtcgacggatgacagctcgcatctacgagctcgagatgacacagagtatcgcatcgagtgcagagcagactcacagctagagatgagtgatcgagctctgctcggcgcgctatagtgagtcgtattacgagtgcagagcagactcac

SEQ ID NO: 4 - DNA, 200 bp, Artificial sequence
  synthetic sequence
caggtacgcagttagcactccgtacgtacgtacgcagctagctcgatgagtactctgctcgatgagtactctgctcgacgagatgagacgagtctctcgtagacgagagcagactcagtcatcgcgctagagagcatagagtcgtgatctatgctcagcgcgctatagtgagtcgtattatccgtagtcatattgccacg

SEQ ID NO: 5 - DNA, 120 bp, Artificial sequence
  synthetic sequence
gggagtaatccccttggcggtcgcgggggacagcgcgtacgtgcgtttaagcggtgctagagctgtctacgaccagcgcgcgctatagtgagtcgtattaggattctccagggcatccgg

SEQ ID NO: 6 - DNA, 23 bp, Artificial sequence
  synthetic sequence
taatacgactcactatagcgcgc

SEQ ID NO: 7 - DNA, 20 bp, Artificial sequence
  synthetic sequence
gtgagtctgctctgcactcg

SEQ ID NO: 8 - DNA, 20 bp, Artificial sequence
  synthetic sequence
cgtggcaatatgactacgga

SEQ ID NO: 9 - DNA, 20 bp, Artificial sequence
  synthetic sequence
ccggatgccctggagaatcc

SEQ ID NO: 10 - DNA, 20 bp, Artificial sequence
  synthetic sequence
ctacgacacagtctctcgcg

SEQ ID NO: 11 - DNA, 20 bp, Artificial sequence
  synthetic sequence
cgtacgtacgtacgtcgacg

SEQ ID NO: 12 - DNA, 20 bp, Artificial sequence
  synthetic sequence
caggtacgcagttagcactc

SEQ ID NO: 13 - DNA, 21 bp, Artificial sequence
  synthetic sequence
gggagtaatccccttggcggt

SEQ ID NO: 14 - DNA, 20 bp, Artificial sequence
  synthetic sequence
cgtacgtacgtacgtcgacg

SEQ ID NO: 15 - DNA, 20 bp, Artificial sequence
  synthetic sequence
gagcagagctcgatcactca

SEQ ID NO: 16 - DNA, 48 bp, Artificial sequence
  synthetic sequence
ctccatcagagtgatatgcccagcttaggtgagtctgctctgcactcg

SEQ ID NO: 17 - DNA, 48 bp, Artificial sequence
  synthetic sequence
cgagtgcagagcagactcacctaagctgggcatatcactctgatggag

SEQ ID NO: 18 - DNA, 40 bp, Artificial sequence
  synthetic sequence
tccgtagtcatattgccacggtgagtctgctctgcactcg

SEQ ID NO: 19 - DNA, 40 bp, Artificial sequence
  synthetic sequence
ggattctccagggcatccgggtgagtctgctctgcactcg

SEQ ID NO: 20 - DNA, 20 bp, Artificial sequence
  synthetic sequence
gtgagtctgctctgcactcg

(19) United States
(12) Patent Application Publication
(10) Pub. No.: US 2022/0028497 A1
(43) Pub. Date: Jan. 27, 2022

(54) METHODS AND SYSTEMS FOR NON-DESTRUCTIVELY STORING, ACCESSING, AND EDITING INFORMATION USING NUCLEIC ACIDS

(71) Applicant: North Carolina State University, Raleigh, NC (US)

(72) Inventors: Albert Jun Qi Keung, Cary, NC (US); James M. Tuck, III, Raleigh, NC (US); Kevin Volkel, Raleigh, NC (US); Kyle Tomek, Raleigh, NC (US); Kevin Lin, Raleigh, NC (US)

(73) Assignee: North Carolina State University, Raleigh, NC (US)

(21) Appl. No.: 17/291,517

(22) PCT Filed: Aug. 30, 2019
(86) PCT No.: PCT/US2019/049170

§ 371 Date: May 5, 2021

Related U.S. Application Data
(60) Provisional application No. 62/756,419, filed on Nov. 6, 2018.

(51) Int. Cl. G16B 50/30; G16B 25/10; G16B 25/20

(52) U.S. Cl. CPC G16B 50/30; G16B 25/20; G16B 25/10

(57) ABSTRACT

Processes and systems for non-destructively storing, accessing, and editing information using nucleic acids are disclosed. Representative processes include a process for extracting a data file from a database, wherein the data file comprises information encoded into one or more polynucleotide strands and wherein the database comprises a plurality of polynucleotide strands; a process for expanding a number of unique data files in a database that can be addressed with a predetermined number of oligonucleotide primers, wherein the unique data files each comprise information encoded into one or more polynucleotide strands and wherein the database comprises a plurality of polynucleotide strands; a process for differentially reading information encoded into one or more polynucleotide strands; a process for manipulating files while in storage; and a process for extracting a data file from a database, wherein the data file comprises information encoded into a polynucleotide strand. Systems for carrying out the processes are also disclosed.

### CROSS-REFERENCE TO RELATED PATENT APPLICATIONS

This application is a U.S. national phase filing based on PCT International Patent Application Serial No. PCT/US2019/049170, filed Aug. 30, 2019, incorporated herein by reference in its entirety, and which claims benefit of U.S. Provisional Application Ser. No. 62/756,419, filed Nov. 6, 2018. The disclosure of this Provisional application is incorporated herein by reference in its entirety.

### GRANT STATEMENT

This invention was made with government support under grant number 1650148 awarded by the National Science Foundation. The government has certain rights in the invention.

### TECHNICAL FIELD

The presently disclosed subject matter relates in some embodiments to methods and systems for non-destructively storing, accessing, and editing information using nucleic acids.

### BACKGROUND

The world's information is rapidly passing zettabyte levels (10²¹ bytes), well beyond the limits of current electronic storage technology. Intriguingly, the biological molecule DNA (deoxyribonucleic acid) has the potential to store zettabyte amounts of information in only a cubic centimeter volume. Furthermore, DNA requires only a small fraction of the energy to store information compared with the large cooling requirements of electronic storage media. However, to achieve extreme capacity DNA-based storage systems, new technologies will be required. In particular, new physical and computational technologies are needed to address challenges that arise specifically from the high density of DNA strands in an extreme scale storage system.

The global datasphere is rapidly surpassing the projected material, space, and energy limits of electronic storage technologies. Reinsel, D., Gantz, J. & Rydning, J. IDC White Pap. Spons. by Seagate 1-25 (2017). DNA features high raw capacity, long-term durability, and minimal energy usage, thus representing a transformative solution as an extreme-scale archival storage medium. While there are limitations centered around the economics of DNA synthesis and sequencing, costs are rapidly decreasing. Carlson, R. Synthesis.com 20-23 (2014). In six years, the field has transitioned from storing and sequencing a 0.69 MB book to a 200 MB database including a music video. Church, G. M., Gao, Y. & Kosuri, S. Science. 337, 1628-1628 (2012); Organick, L. et al. Nat. Biotechnol. 36, 242-249 (2018).

As the feasibility and practicality of DNA storage continues to be determined, new physical and architectural challenges inherent to the development of extreme-scale systems represent an ongoing need in the art.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

Provided in accordance with the presently disclosed subject matter is a process for extracting a data file from a database, wherein the data file comprises information encoded into one or more polynucleotide strands and wherein the database comprises a plurality of polynucleotide strands. In some embodiments, the process comprises: providing an oligonucleotide primer that selectively binds a polynucleotide strand bearing the data file, wherein the primer is labeled with a chemical moiety; contacting the database with the primer and with a magnetic bead comprising a corresponding chemical group that binds the primer moiety; and extracting the one or more polynucleotide strands bearing the data file using a magnet. In some embodiments, the one or more polynucleotide strands comprise a deoxyribonucleic acid (DNA) strand, optionally wherein the DNA strand can be single stranded (ss) or double stranded (ds). In some embodiments, the chemical moiety on the primer and the corresponding chemical group are selected from the group consisting of biotin-streptavidin, fluorescein-antibody, digoxigenin-antibody, and polyA-polyT oligomers.

In some embodiments, the process comprises amplifying the one or more polynucleotide strands bearing the data file prior to extracting the file using the magnet. In some embodiments, the process comprises sequencing the one or more polynucleotide strands bearing the data file. In some embodiments, the process comprises decoding the data file from sequencing data obtained from sequencing the one or more polynucleotide strands bearing the data file; performing error analysis of sequencing data to infer where errors in the process might be occurring and/or the frequency of the errors; or a combination thereof. In some embodiments, the data file can be repeatedly extracted from the same database and wherein the process is a nondestructive process.

Provided in accordance with the presently disclosed subject matter is a process for expanding a number of unique data files in a database that can be addressed with a predetermined number of oligonucleotide primers, wherein the unique data files each comprise information encoded into one or more polynucleotide strands and wherein the database comprises a plurality of polynucleotide strands. In some embodiments, the process comprises designing two or more primers within the predetermined number of oligonucleotide primers that each selectively bind the one or more polynucleotide strands; and assigning a hierarchy to the two or more oligonucleotide primers within the predetermined number of oligonucleotide primers. In some embodiments, the one or more polynucleotide strands comprise a deoxyribonucleic acid (DNA) strand, optionally wherein the DNA strand can be single stranded (ss) or double stranded (ds). In some embodiments, assigning a hierarchy to primers comprises nesting two or more primer binding sites adjacent to a data file to be amplified using an oligonucleotide primer complementary to one of the two or more primer binding sites.

In some embodiments, the process comprises amplifying a data file using a primer for which a hierarchy has been assigned. In some embodiments, the primer binds to one of the two or more primer binding sites. In some embodiments, the primer is labeled with a chemical moiety and the process further comprises contacting the database with the primer and with a magnetic bead comprising a corresponding chemical group that binds the primer moiety; and extracting the one or more polynucleotide strands bearing the data file using a magnet. In some embodiments, the chemical moiety on the primer and the corresponding chemical group are selected from the group consisting of biotin-streptavidin, fluorescein-antibody, digoxigenin-antibody, and polyA-polyT oligomers.

In some embodiments, the process comprises amplifying the polynucleotide strand bearing the data file strand prior to extracting the file using the magnet. In some embodiments, the process comprises sequencing the one or more polynucleotide strands bearing the data file. In some embodiments, the process comprises decoding the data file from sequencing data obtained from sequencing the one or more polynucleotide strands bearing the data file; performing error analysis of sequencing data to infer where errors in the process might be occurring and/or the frequency of the errors; or a combination thereof. In some embodiments, the data file can be repeatedly extracted from the same database and wherein the process is a nondestructive process.

Provided in accordance with the presently disclosed subject matter is a process for differentially reading information encoded into one or more polynucleotide strands. In some embodiments, the process comprises: providing a database comprising a plurality of the polynucleotide strands; providing an oligonucleotide primer that selectively binds one or more polynucleotide strands bearing information; contacting the database with the primer under conditions where the selective binding of the primer to the polynucleotide strand bearing information is controlled; and differentially reading information encoded into the polynucleotide strand based on the binding conditions. In some embodiments, the polynucleotide strand comprises a deoxyribonucleic acid (DNA) strand. In some embodiments, the DNA strand can be single stranded (ss) and/or double stranded (ds).

In some embodiments, the conditions where the selective binding of the primer to the polynucleotide strand bearing information is controlled comprise conditions wherein one or more mis-match interactions between the primer and the polynucleotide strand bearing information occur. In some embodiments, the conditions where the selective binding of the primer to the file is controlled comprise lowering a temperature under which the binding is allowed to proceed, increasing a concentration of primer, varying a binding buffer composition, length of primer, and combinations thereof. In some embodiments, the process comprises amplifying the polynucleotide strand bearing information under the conditions where the selective binding of the primer to the file is controlled.

Provided in accordance with the presently disclosed subject matter is a process for extracting a data file from a database, wherein the data file comprises information encoded into a polynucleotide strand. In some embodiments, the process comprises providing a database comprising a plurality of polynucleotide strands, wherein the data file comprises information encoded into one or more double stranded (ds) polynucleotide strands; providing a physical occlusion that provides selective access to the data file; contacting the database with a reagent that selectively binds a location on one or more of the polynucleotide strands not occluded by physical occlusion; and extracting the one or more polynucleotide strands bearing the data file using the reagent. In some embodiments, the database comprises a plurality of DNA strands, wherein the DNA strands comprise doubled-stranded DNA (dsDNA).

In some embodiments, the physical occlusion comprises a single strand polynucleotide overhang (ss overhang) on the one or more doubled-stranded (ds) polynucleotide strands bearing the data file; and wherein the process comprises: providing an oligonucleotide primer that selectively binds the ss overhang, wherein the primer is labeled with a chemical moiety; contacting the database with the primer and with a magnetic bead comprising a corresponding chemical group that binds the primer moiety; and extracting the one or more polynucleotide strands bearing the data file using a magnet. In some embodiments, the chemical moiety on the primer and the corresponding chemical group are selected from the group consisting of biotin-streptavidin, fluorescein-antibody, digoxigenin-antibody, and polyA-polyT oligomers.

In some embodiments, the ss overhang is hidden by a ss sequence comprising a sequence that is complementary to the ss overhang and a toehold switch sequence, whereby the data file is hidden from extraction; and extracting the one or more polynucleotide strands bearing the data file further comprises contacting the database with a key nucleic acid strand comprising the ss overhang and a sequence complementary to the toehold sequence, whereby the ss overhang on the ds polynucleotide strand is revealed and the labeled primer that selectively binds the ss overhang can bind the ss overhang. In some embodiments, the polynucleotide strand comprises a RNA polymerase promoter sequence, optionally a T7 promoter sequence, and extracting the polynucleotide strand bearing the data file comprises extracting the ss overhang strand using the labeled primer, adding a RNA polymerase to transcribe the polynucleotide strand bearing the data file, and creating ribonucleic acid (RNA), wherein information in the data file can be derived from the RNA. In some embodiments, the extracted polynucleotide strand bearing the data file can be returned to the original database while the RNA is used to derive the data file.

In some embodiments, the physical occlusion comprises a DNA binding molecule, such as but not limited to an archaeal histone proteins, a poly-cationic polymer, a dendrimer, and/or a nucleosome; and wherein the process comprises: providing a reagent that binds a sequence not occluded by the DNA binding molecule, wherein the reagent is labeled with a chemical moiety and wherein the sequence not occluded by the DNA binding molecule is associated with the data file; contacting the database with the reagent and with a magnetic bead comprising a corresponding chemical group that binds the chemical moiety; and extracting the one or more polynucleotide strands bearing the data file using a magnet. In some embodiments, the reagent comprises an oligonucleotide and/or a binding protein, optionally wherein the oligonucleotide comprises a guide RNA and the binding protein comprises dCas9, optionally wherein the oligonucleotide is labeled with a chemical moiety, or optionally wherein the binding protein is a TALE and/or a ZF.

In some embodiments the process comprises sequencing the one or more polynucleotide strands bearing the data file. In some embodiments the process comprises decoding the data file from sequencing data obtained from sequencing the one or more polynucleotide strands bearing the data file; performing error analysis of sequencing data to infer where errors in the process might be occurring and/or the frequency of the errors; or a combination thereof. In some embodiments, the data file can be repeatedly extracted from the same database and wherein the process is a nondestructive process.

Provided in accordance with the presently disclosed subject matter is a system suitable for use in carrying out any of the processes of the presently disclosed subject matter.

Accordingly, it is an object of the presently disclosed subject matter to provide methods and systems for non-destructively storing, accessing, and editing information using nucleic acids. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE DRAWINGS

FIG. 1 is a series of schematic drawings showing how thermodynamics can provide a framework by which to provide solutions and new functions that can be engineered in high capacity and high-density DNA storage systems. The drawing in the upper left shows how thermodynamics can govern DNA-DNA interactions in that the greater the Gibbs free energy of binding (ΔG), the less likely it is that two DNA strands bind. The drawing in the upper right shows how changing temperature can control the amount of mismatch binding that occurs between DNA strands and thus act as a control for data access. Concentration (not shown) can also be used to control the amount of mismatch binding. The drawing in the lower left shows how the energetics of DNA binding can be shifted so that mismatched DNA strands have greater Gibbs free energy. The drawing in the lower right shows how the sequence space of primers can be used with massively parallel measurements of mismatch interactions.

FIG. 2A is a schematic drawing showing the general framework for DNA storage systems. In a first step, digital data is converted into DNA sequences through a choice of encoding schemes. In a second step, the DNA is chemically synthesized. In a third step, all the different DNA strands are mixed into one pool in a solvent and stored in a test tube. To read the data, in a fourth step, many copies of the DNA strands are generated by polymerase chain reaction (PCR) amplification, where a particular file can be read by using PCR primers that amplify a specific set of DNA strands. In fifth and sixth steps, the DNA is read by next generation sequencing and the DNA sequences are decoded.

FIG. 2B is a schematic drawing showing a single DNA strand for DNA storage. DNA storage comprises of many strands of such DNA, typically shorter than 200 base pairs (bp). Data is split across multiple strands, with the order of strands encoding a file specified by an index. Random access is enabled by 20 bp sequences that primers can bind to and the file is amplified by polymerase chain reaction (PCR).

FIG. 3A is a schematic drawing showing a 6000 strand DNA library synthesized comprised of three North Carolina State University logos and two text files.

FIG. 3B is a schematic drawing showing a biotin-streptavidin magnetic extraction that provides specific separation of a file from undesired data.

FIG. 3C is a schematic drawing showing an extraction scheme where the elution is the desired file accessed through biotin-streptavidin linkage, and the supernatant is the retained library.

FIG. 3D is a graph showing the results of error analysis performed on sequencing data to assess the location and frequencies of errors arising from the entire process. The data is shown as the fraction of DNA strands with errors versus sequence location (i.e., base position on the DNA strand, from 0 to 200). Data is plotted for errors before extraction and for all errors.

FIG. 4A is a schematic drawing showing hierarchical primer encoding for two single stranded DNAs of less than 200 nucleotides in length.

FIG. 4B is a graph showing how hierarchy primer encoding exponentially increases the system capacity by nearly 5 orders of magnitude. This provides for specific files of sizes that can be sequenced completely (bottom solid line) to be encoded in an extreme scale, petabyte sized database.

FIG. 5A is a set of graphs showing the fraction of total strands versus sequencing depth for the next generation sequencing of strand distribution from 5 files (File 1, File 2, File 3, File 4, and File 5). Each of the files is comprised of 1000 unique strand sequences.

FIG. 5B is a graph showing the noise versus the number of polymerase chain reaction (PCR) cycles. As indicated by the graph, strand distributions became noisier (more spread out) up to 10 PCR cycles, and then decreases (but remains relatively high), indicating that extraction methods that minimize the number of PCR cycles are desirable. Noise is shown as total noise, noise due to deletions, noise due to insertions, and noise due to substitutions.

FIG. 6A is a schematic diagram showing how temperature and concentration can control which DNA strands are amplified by polymerase chain reaction (PCR).

FIG. 6B is a schematic drawing (top) and an image of a gel (bottom) showing that a DNA architecture that has a 20 base pair (bp) single strand (ss) overhang can physically occlude erroneous strand extraction.

FIG. 7A is a schematic drawing showing a procedure for screening 25 different temperature and concentration conditions for which variant primers bind to their parent sequences. Primers that bind will be adjacent to a constant primer, and will be ligated together, allowing bound primers to be distinguished in next generation sequencing.

FIG. 7B is a schematic drawing showing implementation of a “Preview” procedure. The accessing primer will bind the 100 kilobase (KB) preview strands that have perfect complementarity (left). Only when the temperature is low and primer concentration high will the primer bind the full data strands that have mismatched complementarity (right).

FIG. 8A is a schematic drawing showing that primers can bind many off-target sites in data payload regions in polymerase chain reaction (PCR)-based systems since there is a step that melts double-stranded DNA (dsDNA) into single-stranded DNA (ssDNA).

FIG. 8B is a schematic drawing showing a method to rapidly create many double-stranded DNA (dsDNA) strands with single-stranded DNA (ssDNA) overhangs.

FIG. 8C is a schematic drawing showing DNA wrapped around nucleosome protein complexes that block primers from non-specifically binding data payload regions.

FIG. 8D is a schematic drawing showing how error prone polymerase chain reaction amplification creates mutated variants of a DNA strand.

FIG. 9 is a schematic drawing showing a large library of single-stranded DNA (ssDNA) primers computationally designed and ordered based upon thermodynamic and Hamming/Edit Distance models (top). The primers are allowed to bind to each other to identify binding of mismatched primers (middle). Primers that bind each other will be ligated together and therefore will be read as a single consecutive DNA sequence in next generation sequencing.

FIG. 10A is a pair of gel images showing the effect of holding primer concentration constant and varying temperature.

FIG. 10B is a pair of gel images showing the effect of holding temperature constant and varying primary concentration.

FIG. 11 is a schematic drawing (left) and gel image (right) showing that a specific DNA strand can be extracted without polymerase chain reaction amplification, isothermally, and that specific binding to the middle (or “data payload”) region of the DNA strand can be blocked.

FIG. 12 is series of schematic drawings showing (top left)) a generic framework for DNA-based storage systems that include encoding of digital information to nucleotide sequences, DNA synthesis and storage, DNA sequencing, and decoding of the desired information; (top right) challenges faced by polymerase chain reaction (PCR)-based file access; and (bottom) a procedure (which can be referred to as Dynamic Operations and Reusable Information Storage or DORIS) where a “toehold”-based hybrid DNA provides repeatable information access through non-PCR based magnetic separation, in vitro transcription, reverse transcription, and the return of separated files to the database. Additionally, toeholds unlock in-storage file operations including “lock”, “unlock”, “rename” and “delete.”

FIG. 13 is a schematic drawing of a high level block diagram of a general purpose computer system suitable for use in performing functions described herein

FIG. 14A is a schematic drawing showing the creation of hybrid DNA toehold strands via single primer extension (upper left); a gel image showing that 3 cycles of polymerase chain reaction generate an optimal amount of 160 base pair (bp) hybrid toehold strands while minimizing excess single-stranded DNA production (lower left); and a graph showing the quantification of DBA toehold strands as a function of the ratio of single-stranded DNA:primer as measured by DNA gel fluorescence (in arbitrary units (A.U.).

FIG. 14B is a schematic diagram (top) of a scheme of how individual files can be accessed from a 3-file database created by a “one-pot” single primer extension. Each file was accessed by its corresponding biotin-linked oligo, followed by a non-polymerase chain reaction (PCR) based separation using functionalized magnetic beads. At the bottom a graph shows file access specificity as the percentage of the DNA accessed by a biotin-linked oligo that is either file A, B, or C as measured by quantitative polymerase chain reaction (qPCR).

FIG. 14C is (left) a schematic diagram showing that polymerase chain reaction (PCR) but not Dynamic Operations and Reusable Information Storage (DORIS) allows file access oligos to bind internal off-target sites and produce undesired results; (middle) DNA gels; and (right) the quantified fluorescence from the gels showing that PCR-based access results in truncated and undesired amplicons whereas DORIS accesses only the desired strands.

FIG. 14D is a pair of graphs where the graph at the left shows Monte Carlo simulations estimated the number of oligos found that will not interact with each other or the data payload. 1,000,000 oligos were tested for different density encodings. The x-axis represents density, which is inversely related to the length of “codewords” used to store discrete 1 byte data values. Codeword lengths of 12 to 4 were evaluated. For a polymerase chain reaction (PCR)-based system, longer codewords have lower density but less diverse overall data payload sequences, providing more distinct oligos to be used as addresses in a database. For Dynamic Operations and Reusable Information Storage (DORIS), the encoding density is not impacted because it need not guard against undesired binding between the oligo and data payloads. The graph at the right shows that for PCR, the number of oligos that will not bind the data payload drops as strand density increases, which means that fewer files can be stored, leading to a lower overall system capacity. For DORIS, the availability of oligos is independent of encoding, and capacity therefore, increases with denser encodings.

FIG. 15 is a schematic drawing (left) and graph (right) showing that file access temperature does not appreciably affect access efficiency from a 3-file database created through “one-pot” primer extension. After the 3-file database was created, each file was accessed by its corresponding biotin-linked oligo at 25, 35, or 45° C. and separated using magnetic beads. The amounts of each file in the samples were quantified by quantitative polymerase chain reaction (qPCR). Each oligo accessed its file specifically, and the access/annealing temperature did not appreciably affect the file access efficiency, which was calculated as the amount of DNA accessed relative to its original quantity in the database. Error bars are standard deviations of three replicate file accesses/separations.

FIG. 16A is a schematic drawing showing 5 toehold lengths generated by single primer extension and then evaluated for their file access efficiencies at 5 different temperatures (15, 25, 35, 45, and 55° C.).

FIG. 16B is a heatmap (top) and graph (bottom) showing the experimental analysis of access efficiency for the file access studies described in FIG. 16B. The access efficiency was calculated as the amount of file accessed relative to its starting total quantity as measured by quantitative polymerase chain reaction (qPCR). Error bars are the standard deviations of three replicate file accesses/separations.

FIG. 16C is a graph showing a theoretical analysis of the change in Gibbs free energy at different oligo/toehold lengths at different temperatures.

FIG. 17A is a schematic diagram showing how Dynamic Operations and Reusable Information Storage (DORIS) mimics natural transcription to non-destructively access information. File A was separated using non-polymerase chain reaction (PCR) based magnetic separation while the library was recovered (“Retained Library”). T7-based in vitro transcription was performed directly on the bead-immobilized file for up to 48 hours to generate RNA. Reverse transcription converted the RNA to complementary DNA (cDNA) while the immobilized file A was released back into the database (“Retained File”).

FIG. 17B is a pair of graphs showing the amount of Retained Library and Retained File (after file A was accessed by oligo A′) measured by quantitative polymerase chain reaction (qPCR) and plotted as a percentage of the original amount of each file that was in the database. The specificity of file access is evident by the absence of file B and C in the Retained File. The presence of T7 RNA polymerase (RNAP) did not affect the retention of file A. Error bars are standard deviations of three replicate file accesses.

FIG. 17C is a graph showing the complementary DNA (cDNA) generated from accessed file A amplified by polymerase chain reaction (PCR) to sub-saturating quantities, run on a DNA gel, and quantified by SYBR green fluorescence. IVT=in vitro transcription. RT=reverse transcriptase. Error bars are standard deviations of three replicate file accesses.

FIG. 18A is a pair of graphs showing that the presence/absence of RNA polymerase and in vitro transcription (IVT) time did not affect the retention rate of the Retained Library sample as these samples did not undergo IVT. The presence/absence of RNA polymerase did not affect the retention rate of the Retained File; however, the IVT time did. The decrease in retention rate of the retained file can be partially due to disrupted file-to-bead binding during the elevated temperature of the IVT step. Retention rate is the amount of DNA recovered relative to the starting amount of DNA. Error bars are standard deviations of three replicate IVTs.

FIG. 18B is a pair of graphs showing that a re-annealing step to 45° C. was able to rescue some of the loss described in FIG. 18A. Retention rate is the amount of DNA recovered relative to the starting amount of DNA. Error bars are standard deviations of three replicate in vitro transcriptions (IVTs).

FIG. 19 is a schematic drawing (left) and graph (right) showing that increasing in vitro transcription (IVT) time increases the quantity of RNA produced. IVT was performed directly on file A strands bound to magnetic beads for different lengths of time, with or without RNA polymerase. The amount of RNA polymerase produced was measured directly by a fluorometer. Error bars are standard deviations of three replicate separated files and their corresponding IVTs.

FIG. 20A is a schematic drawing (top) and a graph (bottom) showing how toeholds aid in-storage file operations. The schematic drawing at the top shows “locking” and “unlocking” in-storage file operations. The graph at the bottom shows access efficiency of attempts to access file A by Dynamic Operations and Reusable Information Storage (DORIS) without locking (“No-Lock”); with locking, but without a key (“No-Key”); or with locking and key added at different temperatures. The lock was added at 98° C. The key was added at the different temperatures and then cooled to 14° C. The accessing oligo A′ was added at different access temperatures of 25, 35, 45, or 75° C. for 2 minutes, followed by a temperature drop of 1° C./minute to 25° C. Access efficiency is the amount of file A recovered relative to its original quantity, as measured by quantitative polymerase chain reaction (qPCR). Error bars are standard deviations of three replicate file operations/accesses.

FIG. 20B is a schematic drawing (top) and a graph (bottom) showing how toeholds aid in-storage file operations. The schematic drawing at the top shows “rename” and “delete” in-storage file operations. File A was modified by renaming or deleting oligos. The graph at the bottom shows results of the completion of each operation by measuring how much of the file was accessed by each separate oligo: A′, B′, or C′. Access efficiency is the amount of file A recovered relative to its original quantity, as measured by quantitative polymerase chain reaction (qPCR). “No Mod” refers to no file modification/operation. Error bars are standard deviations of three replicate file operations/accesses.

FIG. 21A is a graph showing how temperature influences the extent of locking. File A was accessed by Dynamic Operations and Reusable Information Storage (DORIS) without locking, or, following provision of a lock, was accessed with or without subsequent unlocking by a key. The lock was added at 45° C. and then cooled to 14° C. The accessing oligo A′ was added at different access temperatures of 25, 35, 45, or 75° C. for 2 minutes, followed by a temperature drop of 1° C./minute to 25° C. Access efficiency is the amount of file A recovered relative to its original quantity, as measured by quantitative polymerase chain reaction (qPCR). Error bars are standard deviations of three replicate file operations/accesses.

FIG. 21B is a graph showing how temperature influences the extent of locking. File A was accessed by Dynamic Operations and Reusable Information Storage (DORIS) without locking, or, following provision of a lock, was accessed with or without subsequent unlocking by a key. The lock was added at 25° C. and then cooled to 14° C. The accessing oligo A′ was added at different access temperatures of 25, 35, 45, or 75° C. for 2 minutes, followed by a temperature drop of 1° C./minute to 25° C. Access efficiency is the amount of file A recovered relative to its original quantity, as measured by quantitative polymerase chain reaction (qPCR). Error bars are standard deviations of three replicate file operations/accesses.

FIG. 22A is a flowchart for estimating viability of a primer implemented as a Python program.

FIG. 22B is a flowchart for producing encoding and decoding tables of varying length.

### DETAILED DESCRIPTION

As the feasibility and practicality of DNA storage continues to be determined, new physical and architectural challenges inherent to the development of extreme-scale systems must be anticipated. Disclosed herein in accordance with aspects of the presently disclosed subject matter are experimental and computational models of these challenges. Further, the presently disclosed subject matter demonstrates how a combination of molecular biology technologies and data encoding strategies can break the barriers to practicality of extreme scale DNA storage systems. In some embodiments, the accessing of a 9 kB file from an overwhelming background of a terabyte quantity of DNA is physically mimicked. In some embodiments, the theoretical information capacity of DNA storage is increased by 10⁵ through new encoding and file access schemes. Furthermore, in some embodiments, molecular strategies and simulations directly address scalability barriers and emulate their solutions. The presently disclosed subject matter supports that DNA storage systems are capable of satisfying the extreme densities and capacities required to fulfill society's projected data storage needs. The presently disclosed subject matter provides processes for manipulating files while in storage.

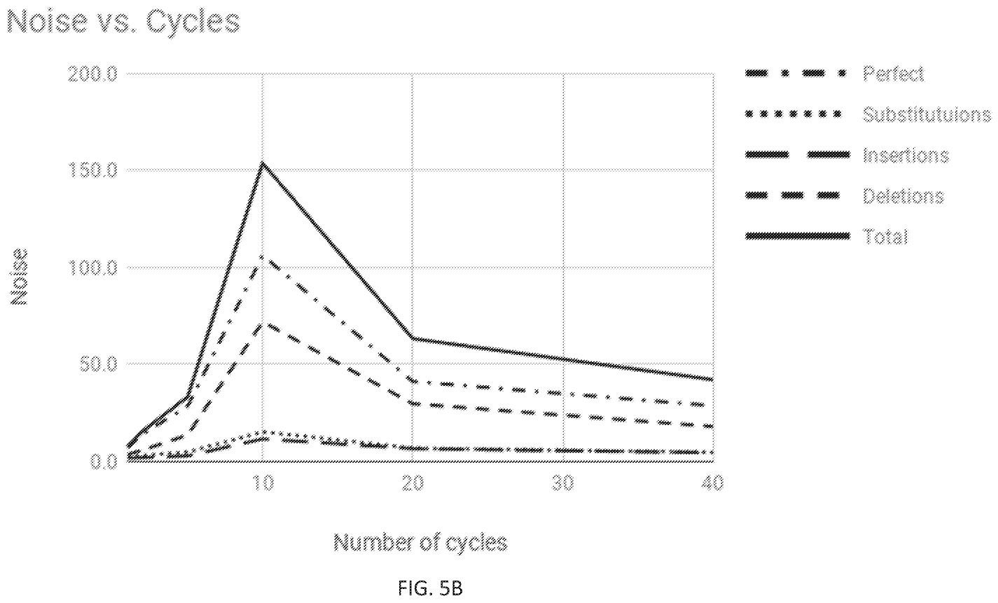

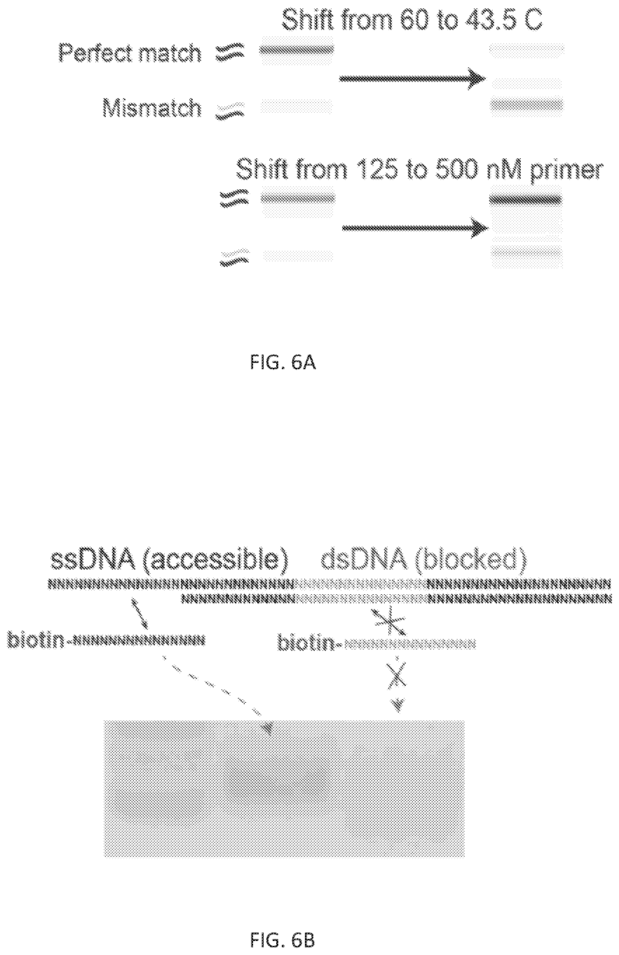

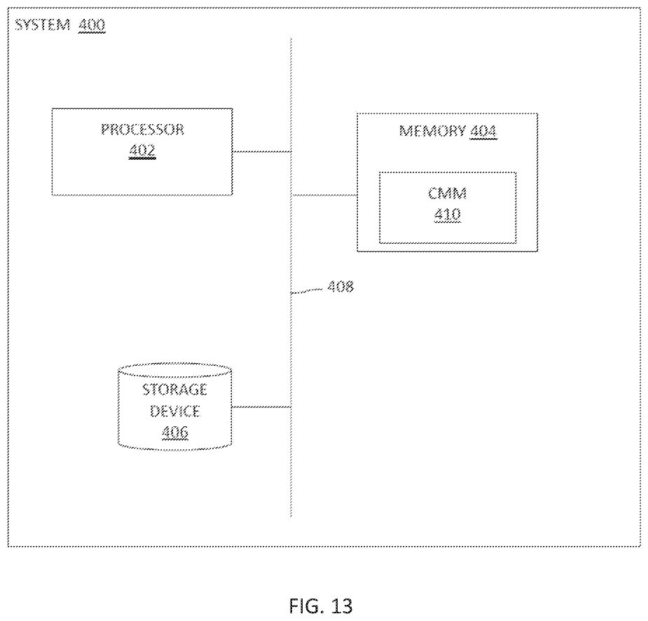

In some embodiments, the presently disclosed subject matter provides a process of extracting a file comprising a set of DNA strands from a large database of many more strands. In some embodiments, the presently disclosed subject matter describes the hierarchical use of primers or file addresses to expand the number of unique files that can be addressed with the same number of total primers. In some embodiments, the presently disclosed subject matter describes how temperature and concentration can be used to differentially read specific subsets of information. In some embodiments, the presently disclosed subject matter describes how a new ssDNA overhang structure on DNA information can be used for isothermal file access while simultaneously preventing non-specific, off-target access of undesired DNA information. See FIGS. 6B, 11, and 14C.

Current electronic storage technology is much less information dense than nucleic acid polymers such as DNA. It requires significant energy usage to maintain compared to DNA. It also has poor stability, often less than a decade, compared to DNA which can last hundreds of year. These advantages of DNA suggest its use as a highly efficient storage technology for archival (low access) data. The field of DNA-based storage is young and has focused on schemes to encode information into DNA. The field has not yet addressed the many both physical and computational problems inherent in truly extreme capacity DNA-based storage. In particular, from both economic and technological standpoints, DNA synthesis and sequencing technologies cannot work with DNA databases greater than several gigabytes (10⁹ bytes) unless a technology is invented to access and separate out specific subsets of data of a size manageable for DNA sequencing without destroying the original database. The presently disclosed subject matter provides this capability.

The presently disclosed subject matter provides for the extraction of a small file from a very large background. In one representative, non-limiting example, 0.01% of an entire database was effectively extracted successfully. While the background strands that were used were not all unique (due to expense to synthesize), 1 gigabyte of DNA strands was effectively extracted from 10 terabytes of DNA strands. If it were currently financially feasible to order 10¹² completely distinct and unique DNA strands, the presently disclosed processes and systems would be able to execute file manipulations on the order of contemporary electronic devices from an overall database size exceeding contemporary electronic devices.

Possible uses for the presently disclosed subject matter includes information storage systems for the long-term archival storage of information, such as old photos, texts, literature, music, and videos. Other uses of this technology could be in improving the manipulation and separation of DNA molecules in molecular biology processes and bioengineering applications.

The presently disclosed subject matter provides experimental and computational models that address physical and architectural challenges inherent to the development of extreme-scale systems. The presently disclosed subject matter demonstrates how a combination of molecular biology technologies and data encoding strategies can break the barriers to practicality of extreme scale DNA storage systems. In one example, the accessing of a 9 kB file from an overwhelming background of a terabyte quantity of DNA is mimicked, supporting the increase of the theoretical information capacity of DNA storage by 10⁵ through new encoding and file access schemes. Furthermore, the presently disclosed subject matter describes how molecular strategies and simulations directly address scalability barriers and emulate their solutions. The presently disclosed subject matter supports that DNA storage systems are capable of satisfying the extreme densities and capacities required to fulfill society's projected data storage needs.

### Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

While the following terms are believed to be well understood by one of ordinary skill in the art, the following definitions are set forth to facilitate explanation of the presently disclosed subject matter.

As used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like, in connection with the recitation of claim elements, or use of a “negative” limitation.

Ranges can be expressed herein as from “about” one particular value, and/or to “about” another particular value. When such a range is expressed, some embodiments includes from the one particular value and/or to the other particular value. Similarly, when values are expressed as approximations, by use of the antecedent “about,” it will be understood that the particular value forms an embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” are also disclosed. It is also understood that the throughout the application, data are provided in a number of different formats, and that these data represent in some embodiments endpoints and starting points and in some embodiments ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “and/or”, when used in the context of a list of entities, refers to the entities being present singly or in combination.

The terms “optional” and “optionally” as used herein indicate that the subsequently described event, circumstance, element, and/or method step may or may not occur and/or be present, and that the description includes instances where said event, circumstance, element, or method step occurs and/or is present as well as instances where it does not.

As used herein, the terms “complement,” “complementary,” “complementarity,” and the like, refer to the capacity for precise pairing between nucleobases in an oligonucleotide primer and nucleobases in a target sequence. Thus, if a nucleobase (e.g., adenine) at a certain position of an oligonucleotide primer is capable of hydrogen bonding with a nucleobase (e.g., thymidine, uracil) at a certain position in a target sequence in a target nucleic acid, then the position of hydrogen bonding between the oligonucleotide primer and the target nucleic acid is considered to be a complementary position. Usually, the terms complement, complementary, complementarity, and the like, are viewed in the context of a comparison between a defined number of contiguous nucleotides in a first nucleic acid molecule (e.g., an oligonucleotide primer) and a similar number of contiguous nucleotides in a second nucleic acid molecule (e.g., a DNA molecule bearing a data file in a database), rather than in a single base to base manner. For example, if an oligonucleotide primer is 25 nucleotides in length, its complementarity with a target sequence is usually determined by comparing the sequence of the entire oligonucleotide primer, or a defined portion thereof, with a number of contiguous nucleotides in a target molecule. An oligonucleotide primer and a target sequence are complementary to each other when a sufficient number of corresponding positions in each molecule are occupied by nucleobases which can hydrogen bond with each other. Positions are corresponding when the bases occupying the positions are spatially arranged such that, if complementary, the bases form hydrogen bonds. As an example, when comparing the sequence of an oligonucleotide primer to a similarly sized sequence in a target sequence, the first nucleotide in the oligonucleotide primer is compared with a chosen nucleotide at the start of the target sequence. The second nucleotide in the oligonucleotide primer (3′ to the first nucleotide) is then compared with the nucleotide directly 3′ to the chosen start nucleotide. This process is then continued with each nucleotide along the length of the oligonucleotide primer. Thus, the terms “specifically hybridizable”, “selectively hybridizable”, and “complementary” are terms which are used to indicate a sufficient degree of precise pairing or complementarity over a sufficient number of contiguous nucleobases such that stable and specific binding occurs between the oligonucleotide primer and a target nucleic acid.

Hybridization conditions under which a first nucleic acid molecule will specifically hybridize with a second nucleic acid molecule are commonly referred to in the art as stringent hybridization conditions. It is understood by those skilled in the art that stringent hybridization conditions are sequence-dependent and can be different in different circumstances. Thus, stringent conditions under which an oligonucleotide primer of this disclosure specifically hybridizes to a target sequence are determined by the complementarity of the oligonucleotide primer sequence and the target sequence and the nature of the assays in which they are being investigated. Upon a review of the instant disclosure, persons skilled in the relevant art are capable of designing complementary sequences that specifically hybridize to a particular target sequence for a given assay or a given use. Particular variations of hybridization conditions can be modified in accordance with aspects of the presently disclosed subject matter for accessing data files.

Once a target sequence has been identified, the oligonucleotide primer is designed to include a nucleobase sequence sufficiently complementary to the target sequence so that the oligonucleotide primer specifically hybridizes to the target nucleic acid. More specifically, the nucleotide sequence of the oligonucleotide primer is designed so that it contains a region of contiguous nucleotides sufficiently complementary to the target sequence so that the oligonucleotide primer specifically hybridizes to the target nucleic acid. Such a region of contiguous, complementary nucleotides in the oligonucleotide primer can be referred to as an “antisense sequence” or a “targeting sequence.”

It is well known in the art that the greater the degree of complementarity between two nucleic acid sequences, the stronger and more specific is the hybridization interaction. It is also well understood that the strongest and most specific hybridization occurs between two nucleic acid molecules that are fully complementary. As used herein, the term fully complementary refers to a situation when each nucleobase in a nucleic acid sequence is capable of hydrogen binding with the nucleobase in the corresponding position in a second nucleic acid molecule. In some embodiments, the targeting sequence is fully complementary to the target sequence. In some embodiments, the targeting sequence comprises an at least 6 contiguous nucleobase region that is fully complementary to an at least 6 contiguous nucleobase region in the target sequence. In some embodiments, the targeting sequence comprises an at least 8 contiguous nucleobase sequence that is fully complementary to an at least 8 contiguous nucleobase sequence in the target sequence. In some embodiments, the targeting sequence comprises an at least 10 contiguous nucleobase sequence that is fully complementary to an at least 10 contiguous nucleobase sequence in the target sequence. In some embodiments, the targeting sequence comprises an at least 12 contiguous nucleobase sequence that is fully complementary to an at least 12 contiguous nucleobase sequence in the target sequence. In some embodiments, the targeting sequence comprises an at least 14 contiguous nucleobase sequence that is fully complementary to an at least 14 contiguous nucleobase sequence in the target sequence. In some embodiments, the targeting sequence comprises an at least 16 contiguous nucleobase sequence that is fully complementary to an at least 16 contiguous nucleobase sequence in the target sequence. In some embodiments, the targeting sequence comprises an at least 18 contiguous nucleobase sequence that is fully complementary to an at least 18 contiguous nucleobase sequence in the target sequence. In some embodiments, the targeting sequence comprises an at least 20 contiguous nucleobase sequence that is fully complementary to an at least 20 contiguous nucleobase sequence in the target sequence.

It will be understood by those skilled in the art that the targeting sequence may make up the entirety of an oligonucleotide primer of this disclosure, or it may make up just a portion of an oligonucleotide primer of this disclosure. For example, in an oligonucleotide primer consisting of 30 nucleotides, all 30 nucleotides can be complementary to a 30 contiguous nucleotide target sequence. Alternatively, for example, only 20 contiguous nucleotides in the oligonucleotide primer may be complementary to a 20-contiguous nucleotide target sequence, with the remaining 10 nucleotides in the oligonucleotide primer being mismatched to nucleotides outside of the target sequence. In some embodiments, oligonucleotide primers of this disclosure have a targeting sequence of at least 10 nucleobases, at least 11 nucleobases, at least 12 nucleobases, at least 13 nucleobases, at least 14 nucleobases, at least 15 nucleobases, at least 16 nucleobases, at least 17 nucleobases, at least 18 nucleobases, at least 19 nucleobases, at least 20 nucleobases, at least 21 nucleobases, at least 22 nucleobases, at least 23 nucleobases, at least 24 nucleobases, at least 25 nucleobases, at least 26 nucleobases, at least 27 nucleobases, at least 28 nucleobases, at least 29 nucleobases, or at least 30 nucleobases in length.

In accordance with some embodiments of the presently disclosed subject matter, the inclusion of mismatches between a targeting sequence and a target sequence is possible without eliminating the functionality of the oligonucleotide primer. Moreover, such mismatches can occur anywhere within the interaction between the targeting sequence and the target sequence, so long as the oligonucleotide primer is capable of specifically hybridizing to the targeted nucleic acid molecule. Thus, oligonucleotide primers of this disclosure may comprise up to about 50% nucleotides that are mismatched, thereby disrupting base pairing of the oligonucleotide primer to a target sequence, as long as the oligonucleotide primer specifically hybridizes to the target sequence. In some embodiments, oligonucleotide primers comprise no more than 50%, no more than 45%, no more than 40%, no more than 35%, no more than 30%, no more than 25%, no more than 20%, no more than about 15%, no more than about 10%, no more than about 5% or not more than about 3% of mismatches, or less. In some embodiments, there are no mismatches between nucleotides in the oligonucleotide primer involved in pairing and a complementary target sequence. In some embodiments, mismatches do not occur at contiguous positions. For example, in an oligonucleotide primer containing 3 mismatch positions, in some embodiments the mismatched positions can be separated by runs (e.g., 3, 4, 5, etc.) of contiguous nucleotides that are complementary with 15 nucleotides in the target sequence.

The use of percent identity is a common way of defining the number of mismatches between two nucleic acid sequences. For example, two sequences having the same nucleobase pairing capacity would be considered 100% identical. Moreover, it should be understood that both uracil and thymidine will bind with adenine. Consequently, two molecules that are otherwise identical in sequence would be considered identical, even if one had uracil at position x and the other had a thymidine at corresponding position x. Percent identity may be calculated over the entire length of the oligomeric compound, or over just a portion of an oligonucleotide primer. For example, the percent identity of a targeting sequence to a target sequence can be calculated to determine the capacity of an oligonucleotide primer comprising the targeting sequence to bind to a nucleic acid molecule comprising the target sequence. In some embodiments, the targeting sequence is at least 80% identical, at least 85% identical, at least 90% identical, at least 95% identical, at least 97% identical, at least 98% identical or at least 99% identical over its entire length to a target sequence in a target nucleic acid molecule. In some embodiments, the targeting sequence is identical over its entire length to a target sequence in a target nucleic acid molecule. It is understood by those skilled in the art that an oligonucleotide primer need not be identical to the oligonucleotide primer sequences disclosed herein to function similarly to the oligonucleotide primers described herein. Shortened versions of oligonucleotide primers taught herein, or non-identical versions of the oligonucleotide primers taught herein, fall within the scope of this disclosure. Non-identical versions are those wherein each base does not have 100% identity with the oligonucleotide primers disclosed herein. Alternatively, a non-identical version can include at least one base replaced with a different base with different pairing activity (e.g., G can be replaced by C, A, or T). Percent identity is calculated according to the number of bases that have identical base pairing corresponding to the oligonucleotide primer to which it is being compared. The non-identical bases may be adjacent to each other, dispersed throughout the oligonucleotide primer, or both. For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 18-mer is 78% identical to the 18-mer. Such calculations are well within the ability of those skilled in the art. Thus, oligonucleotide primers of this disclosure comprise oligonucleotide sequences at least 80% identical, at least 85% identical, at least 90% identical, at least 92% identical, at least 94% identical at least 96% identical or at least 98% identical to sequences disclosed herein, as long as the oligonucleotide primers are able to bind and/or amplify a given target sequence.

Before the present compounds, compositions, articles, devices, and/or processes are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Processes of the Presently Disclosed Subject Matter

In some embodiments, the presently disclosed subject provides a process for extracting a data file from a database, wherein the data file comprises information encoded into one or more polynucleotide strands and wherein the database comprises a plurality of polynucleotide strands. In some embodiments the process comprises providing an oligonucleotide primer that selectively binds one or more polynucleotide strands bearing the data file; and extracting the one or more polynucleotide strands bearing the data file. In some embodiments, the primer is labeled with a chemical moiety and the process comprises contacting the database with the primer and with a magnetic bead comprising a corresponding chemical group that binds the primer moiety; and extracting the one or more polynucleotide strands bearing the data file using a magnet. In some embodiments, files can be repeatedly extracted from the same database and the process is a nondestructive process.

In some embodiments, the one or more polynucleotide strands comprise a deoxyribonucleic acid (DNA) strand. The DNA strand can be single stranded (ss) and/or double stranded (ds). In some embodiments, the chemical moiety on the primer and the corresponding chemical group are selected from the group comprising biotin-streptavidin, fluorescein-antibody, digoxigenin-antibody, and polyA-polyT oligomers, and combinations thereof. See Table 1. In some embodiments, the method comprises amplifying the one or more polynucleotide strands bearing the data file prior to extracting the file using the magnet.

TABLE 1

| | Primer 1 | Primer 2 | Primer 3 | Primer 4 | Primer 5 |
|---|---|---|---|---|---|
| Sample | qPCR | qPCR | qPCR | qPCR | qPCR |
| File 1 Biotin elution | 94% | 1% | 1% | 3% | 2% |
| File 1 Biotin supe | 32% | 16% | 19% | 18% | 15% |
| File 2 Biotin elution | 1% | 95% | 0% | 2% | 2% |
| File 2 Biotin supe | 6% | 29% | 11% | 24% | 29% |
| File 3 Biotin elution | 1% | 2% | 92% | 3% | 3% |
| File 3 Biotin supe | 5% | 13% | 41% | 22% | 20% |
| File 2 FL eluiton | 0% | 85% | 0% | 8% | 7% |
| File 2 FL supe | 6% | 10% | 8% | 34% | 43% |
| File 3 DIG elution | 0% | 0% | 88% | 6% | 6% |
| File 3 DIG supernatant | 5% | 5% | 9% | 38% | 43% |

In some embodiments, the process comprises sequencing the one or more polynucleotide strands bearing the data file. In some embodiments, the process comprises decoding the data file from sequencing data obtained from sequencing the one or more polynucleotide strands bearing the data file; performing error analysis of sequencing data to infer where errors in the process might be occurring and/or the frequency of the errors; or a combination thereof. In some embodiments, the other remaining data files are still left available for future access (see ‘supe’ or ‘supernatant’ in Table 1).

Provided in accordance with some embodiments of the present disclosed subject matter are processes for expanding a number of unique data files in a database that can be addressed with a predetermined number of oligonucleotide primers, wherein the unique data files each comprise information encoded into one or more polynucleotide strands and wherein the database comprises a plurality of polynucleotide strands. In some embodiments, the process comprises designing two or more primers within the predetermined number of oligonucleotide primers that each selectively bind the one or more polynucleotide strands; and assigning a hierarchy to the two or more oligonucleotide primers within the predetermined number of oligonucleotide primers. In some embodiments, the polynucleotide strand comprises a deoxyribonucleic acid (DNA) strand. The DNA strand can be single stranded (ss) and/or double stranded (ds).

In some embodiments, assigning a hierarchy to primers comprises nesting two or more primer binding sites adjacent to a data file to be amplified using an oligonucleotide primer complementary to one of the two or more primer binding sites. In some embodiments, the process comprises amplifying a data file using a primer for which a hierarchy has been assigned, optionally wherein the primer binds to one of the two or more primer binding sites. In some embodiments, one amplification is with the first primer in a hierarchy, then extraction, another amplification is done with a second primer, then by a third if designed with 3 primer binding sites, 4 if designed with 4 primer binding sites (etc.). See Table 2.

TABLE 2

| Sample name | 1st primer | Separation? | 2nd Primer | Separation? | File 1 | File 2 | File 3 | primer 4 | primer 5 |
|---|---|---|---|---|---|---|---|---|---|
| Working library | — | — | — | — | 17.7% | 24.8% | 29.9% | 12.2% | 15.4% |
| p4 control | p4 | — | — | — | 27.6% | 20.0% | 23.2% | 10.7% | 18.4% |
| p4B elution | p4B | Y | — | — | 38.1% | 16.5% | 8.4% | 18.9% | 18.1% |
| p4B supernatant | p4B | Y | — | — | 28.6% | 20.0% | 23.5% | 9.4% | 18.5% |
| p4 p5 control | p4 | — | p5 | — | 0.1% | 0.0% | 0.0% | 19.1% | 80.7% |
| p4B elution p5 control | p4B | Y | p5 | — | 0.0% | 0.0% | 0.0% | 3.0% | 96.9% |
| p4B elution p5 elution | p4B | Y | p5FL | Y | 0.0% | 0.0% | 0.0% | 2.0% | 98.0% |
| p4B elution p5 supernatant | p4B | Y | p5FL | Y | 0.0% | 0.0% | 0.0% | 2.0% | 97.9% |
| p4 p4 control | p4 | — | p4 | — | 0.1% | 0.1% | 0.0% | 68.8% | 31.0% |
| p4B elution p4 control | p4B | Y | p4 | — | 0.0% | 0.0% | 0.0% | 61.8% | 38.2% |
| p4B elution p4 elution | p4B | Y | p4FL | Y | 0.1% | 0.1% | 0.0% | 56.4% | 43.4% |
| p4B elution p4 supernatant | p4B | Y | p4FL | Y | 0.1% | 0.1% | 0.0% | 60.8% | 39.0% |
| Working library | — | — | — | — | 17.7% | 24.8% | 29.9% | 12.2% | 15.4% |
| p5 control | p5 | — | — | — | 31.1% | 19.1% | 17.9% | 9.6% | 22.3% |
| p5B elution | p5B | Y | — | — | 43.6% | 19.9% | 7.3% | 8.0% | 21.2% |
| p5B supernatant | p5B | Y | — | — | 30.9% | 18.2% | 21.6% | 8.5% | 20.7% |
| p5 p4 control | p5 | — | p4 | — | 0.1% | 0.1% | 0.0% | 71.2% | 28.5% |
| p5B elution p4 control | p5B | Y | p4 | — | 0.3% | 0.1% | 0.1% | 86.8% | 12.7% |
| p5B elution p4 elution | p5B | Y | p4FL | Y | 0.1% | 0.1% | 0.0% | 86.5% | 13.3% |
| p5B elution p4 supernatant | p5B | Y | p4FL | Y | 0.0% | 0.1% | 0.0% | 85.4% | 14.5% |
| p5 p5 control | p5 | — | p5 | — | 0.1% | 0.0% | 0.0% | 18.5% | 81.4% |
| p5B elution p5 control | p5B | Y | p5 | — | 0.1% | 0.0% | 0.0% | 20.1% | 79.8% |
| p5B elution p5 elution | p5B | Y | p5FL | Y | 0.1% | 0.7% | 0.0% | 19.6% | 79.6% |
| p5B elution p5 supernatant | p5B | Y | p5FL | Y | 0.1% | 0.1% | 0.0% | 19.5% | 80.3% |

In some embodiments, the primer is labeled with a chemical moiety and the process further comprises contacting the database with the primer and with a magnetic bead comprising a corresponding chemical group that binds the primer moiety; and extracting the polynucleotide strands bearing the data file using a magnet. In some embodiments, the chemical moiety on the primer and the corresponding chemical group are selected from the group consisting of biotin-streptavidin, fluorescein-antibody, digoxigenin-antibody, and polyA-polyT oligomers. In some embodiments, the method comprises amplifying the one or more polynucleotide strands bearing the data file prior to extracting the file using the magnet.

In some embodiments, the process comprises sequencing the one or more polynucleotide strands bearing the data file. In some embodiments, the process comprises decoding the data file from sequencing data obtained from sequencing the one or more polynucleotide strands bearing the data file; performing error analysis of sequencing data to infer where errors in the process might be occurring and/or the frequency of the errors; or a combination thereof.

Provided in accordance with some embodiments of the present disclosed subject matter are processes for differentially reading information encoded into one or more polynucleotide strands. In some embodiments, the processes comprise: providing a database comprising a plurality of the polynucleotide strands; providing an oligonucleotide primer that selectively binds one or more polynucleotide strands bearing information; contacting the database with the primer under conditions where the selective binding of the primer to the polynucleotide strand bearing information is controlled; and differentially reading information encoded into the polynucleotide strand based on the binding conditions. In some embodiments, the one or more polynucleotide strands comprise a deoxyribonucleic acid (DNA) strand. The DNA strand can be single stranded (ss) and/or double stranded (ds).

In some embodiments, the conditions where the selective binding of the primer to the polynucleotide strand bearing information is controlled comprise conditions wherein one or more mis-match interactions between the primer and the polynucleotide strand bearing information occur. In some embodiments, the conditions where the selective binding of the primer to the file is controlled comprise lowering a temperature under which the binding is allowed to proceed, increasing a concentration of primer, varying a binding buffer composition, length of primer, and combinations thereof. Representative conditions are disclosed in the Examples. Representative conditions also include addition of dimethylsulfoxide, betaine, MgCl₂, and template concentration. In some embodiments, the process comprises amplifying the polynucleotide strand bearing information under the conditions where the selective binding of the primer to the file is controlled.

Provided in accordance with some embodiments of the present disclosed subject matter are processes for extracting a data file from a database, wherein the data file comprises information encoded into one or more polynucleotide strands. In some embodiments, the process comprises blocking access to the data file. In some embodiments, blocking access to the data file comprises providing a physical occlusion that prevents primers from binding off-target sequences non-specifically. As described more fully herein, one approach involves overhangs and another approach involves a nucleosome-based approach. In some embodiments, nucleosomes are used to block access of primers to offtarget sites. This approach can particularly be used in accessing data from fully double-stranded DNA, although nucleosomes can enhance blocking offtarget sites in conjunction with the ssDNA overhang structures. Further, in some embodiments, upon realizing primers will not work in the situation of using nucleosomes because necessary increases in temperature to allow primer melting and binding would denature the nucleosomes, biotin-labeled dCas9 protein or other sequence programmable DNA-binding protein is used to pull out a data file, instead of primers. By way of further exemplification and not limitation, this approach is employed with dsDNA coiled around nucleosomes.

Thus, in some embodiments, a process for extracting a data file from a database is provided. In some embodiments, the data file comprises information encoded into a polynucleotide strand and the process comprises providing a database comprising a plurality of polynucleotide strands, wherein the data file comprises information encoded into one or more polynucleotide strands, optionally one or more double stranded (ds) polynucleotide strands; providing a physical occlusion that provides selective access to the data file; contacting the database with a reagent that selectively binds at a location on one or more of the polynucleotide strands not occluded by the physical occlusion; and extracting the one or more polynucleotide strands bearing the data file using the reagent. In some embodiments, the database comprises a plurality of DNA strands, wherein the DNA strands comprise doubled-stranded DNA (dsDNA).

In some embodiments, the physical occlusion comprises a single strand polynucleotide overhang (ss overhang) on the one or more doubled-stranded (ds) polynucleotide strands bearing the data file; and the process comprises: providing an oligonucleotide primer that selectively binds the ss overhang; and extracting the one or more polynucleotide strands bearing the data file. In some embodiments, the primer is labeled with a chemical moiety and the process comprises contacting the database with the primer and with a magnetic bead comprising a corresponding chemical group that binds the primer moiety; and extracting the one or more polynucleotide strands bearing the data file using a magnet. In some embodiments, the chemical moiety on the primer and the corresponding chemical group are selected from the group comprising biotin-streptavidin, fluorescein-antibody, digoxigenin-antibody, and polyA-polyT oligomers.

In some embodiments, the ss overhang is hidden by a ss sequence comprising a sequence that is complementary to the ss overhang and a toehold switch sequence, whereby the data file is hidden from extraction; and extracting the one or more polynucleotide strands bearing the data file further comprises contacting the database with a key nucleic acid strand comprising the ss overhang and a sequence complementary to the toehold sequence, whereby the ss overhang on the ds polynucleotide strand is revealed and the labeled primer that selectively binds the ss overhang can bid the ss overhang.

In some embodiments, a sequence adjacent to the ss overhang comprises a RNA promoter sequence, such as a T7 promoter sequence. A representative configuration is as follows: [ssOverhang] [RNA polymerase promoter] [index-data]. In some embodiments, the process can further comprise extracting the polynucleotide strand bearing the data file. In some embodiments, the extracting comprises extracting the ss overhang strand using the labeled primer, adding RNA polymerase, such as but not limited to T7 RNA polymerase, to transcribe the polynucleotide strand bearing the data file, and creating ribonucleic acid (RNA), wherein information in the data file can be derived from the RNA. In some embodiments, the extracted polynucleotide strand bearing the data file can be returned to the original database while the RNA is used to derive the data file.

By way of further exemplification and not limitation, “toehold switches” from synthetic biology are used to create “Hidden Files”. In some embodiments provided is one of the overhang file strand structures with overhang sequence 1′. A “toehold switch” strand is added and the toehold switch comprises toehold sequence 2 and a sequence 1 that is complementary to 1′. When this strand is added, the system will not be able to extract the file, even with a biotin primer with sequence 1. However, if one adds a “key strand”, which is composed of 2′ and 1′, the 2′ will bind the toehold sequence 2, it will unravel the toehold switch strand from the overhang file strand based on thermodynamics, and “unlock” or unblock the overhang file strand. Now the overhang file strand can be accessed by a biotin-primer method, for example as disclosed elsewhere herein.

In some embodiments, when creating the overhang structure, the primer is designed so that the sequence directly adjacent to the overhang structure is the same as a T7 promoter. This will allows a T7 RNA polymerase to be added so that RNA can be transcribed from the strand. In some embodiments, the workflow is: extract overhang strand using biotin-labeled primer, add T7 RNA polymerase to transcribe the strands, and create RNA. An advantage here is that the biotin-extracted overhang structures can then be put back into the original library, and the RNA can then be used in the sequencing. Other RNA polymerases that can be used include T3 RNA polymerase and SP6 RNA polymerase. An additional system that could be used to further expand the number of distinct sequences that transcription can initiate from (increase number of different RNA polymerase promoter sequences): use E. coli RNA polymerase core enzyme and add in different types of sigma factors that will make the E. coli RNA polymerase specifically bind and transcribe from different promoter sequences that depend on the specific sigma factor added.

In some embodiments, the physical occlusion comprises a DNA binding molecule or complex of molecules, such as but not limited to an archaeal histone proteins, a poly-cationic polymer, a dendrimer (such as but not limited polyethylenimine), and/or a nucleosome; and wherein the process comprises providing a reagent that binds a sequence not occluded by the DNA binding molecule or complex of molecules; and extracting the one or more polynucleotide strands bearing the data file. In some embodiments, the reagent is labeled with a chemical moiety and wherein the sequence not occluded by the DNA binding molecule or complex of molecules is associated with the data file and the process comprises contacting the database with the reagent and with a magnetic bead comprising a corresponding chemical group that binds the chemical moiety; and extracting the one or more polynucleotide strands bearing the data file using a magnet. In some embodiments, the reagent comprises an oligonucleotide and/or a binding protein, optionally wherein the oligonucleotide comprises a guide RNA and the binding protein comprises dCas9, optionally wherein the oligonucleotide is labeled with a chemical moiety, or optionally wherein the binding protein is a TALE and/or a ZF. dCas12a with its gRNA is another example. Additional examples include transcription activator like effectors and zinc fingers (TALEs and ZFs), which do not use gRNAs, but themselves are proteins that can be engineered to bind specific DNA sequences.

In some embodiments, the process comprises sequencing the one or more polynucleotide strands bearing the data file. In some embodiments, the process comprises decoding the data file from sequencing data obtained from sequencing the one or more polynucleotide strands bearing the data file; performing error analysis of sequencing data to infer where errors in the process might be occurring and/or the frequency of the errors; or a combination thereof. In some embodiments, the data file can be repeatedly extracted from the same database and wherein the process is a nondestructive process. Thus, the ability to repeatedly extract the same file as well as different files from the same database sample has been demonstrated. This further demonstrates the reusability of the DNA material, cutting down on the need to resynthesize DNA.

### Systems of the Presently Disclosed Subject Matter

Disclosed herein in accordance with some embodiments of the presently disclosed subject matter are systems suitable for use in carrying out any of the processes set forth elsewhere herein. For example, several systems are disclosed in the Figures.

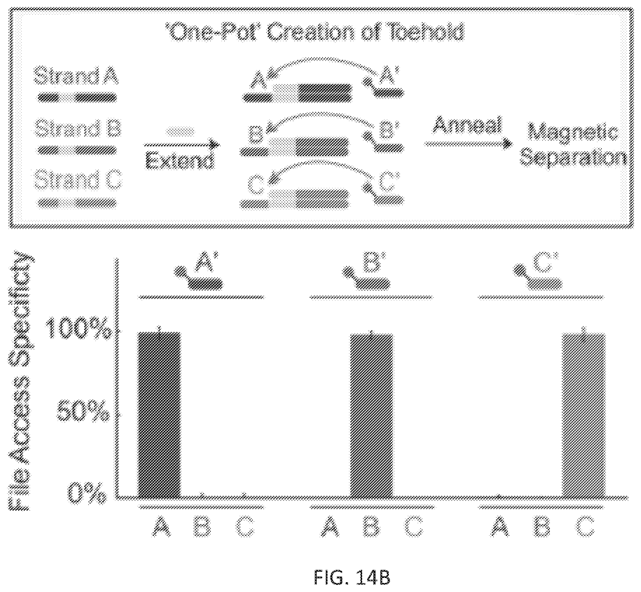

By way of exemplification and not limitation, FIG. 13 depicts a high level block diagram of a general purpose computer system suitable for use in performing functions described herein. As depicted in FIG. 13, system 400 comprises a processor 402, a memory 404, a storage device 406, and communicatively connected via a system bus 408. In some embodiments, processor 402 can comprise a microprocessor, central processing unit (CPU), or any other like hardware based processing unit. In some embodiments, a CMM 410 can be stored in memory 404, which can comprise random access memory (RAM), read only memory (ROM), optical read/write memory, cache memory, magnetic read/write memory, flash memory, or any other non-transitory computer readable medium. In some embodiments, processor 402 and memory 404 can be used to execute and manage the operation of CMM 410. In some embodiments, storage device 406 can comprise any storage medium or storage unit that is configured to store data accessible by processor 402 via system bus 408. Exemplary storage devices can comprise one or more local databases hosted by system 400.

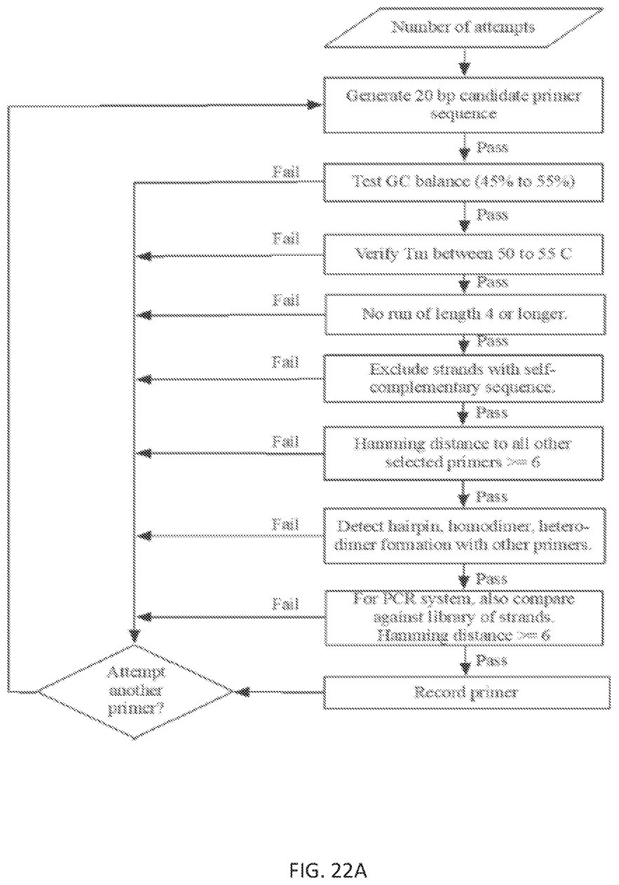

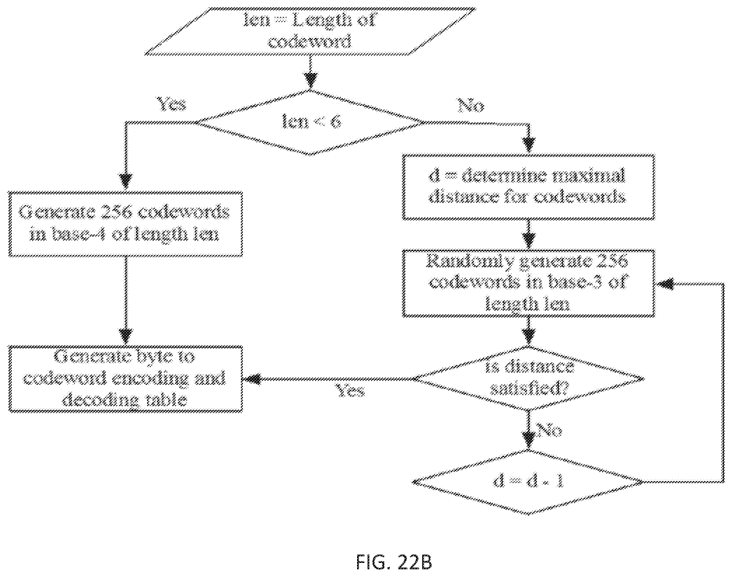

As indicated above, the subject matter described herein can be implemented in software in combination with hardware and/or firmware. For example, the subject matter described herein can be implemented in software executed by a processor. In one exemplary implementation, the subject matter described herein can be implemented using a computer readable medium having stored thereon computer executable instructions that, when executed by a processor of a computer, control the computer to perform steps. Exemplary computer readable mediums suitable for implementing the subject matter described herein include non-transitory devices, such as disk memory devices, chip memory devices, programmable logic devices, and application specific integrated circuits. In addition, a computer readable medium that implements the subject matter described herein can be located on a single device or computing platform or can be distributed across multiple devices or computing platforms. As used herein, the term “module” refers to hardware, firmware, or software in combination with hardware and/or firmware for implementing features described herein. See also FIGS. 22A and 22B.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Introduction

The concept of storing information in DNA first arose in the last two decades of the 20^{th} century. In fact, the idea of a storage system comprising many distinct ˜100-200 base pair (bp) DNA strands, with content addressable through ˜20 bp-long unique DNA sequences on each strand, still serves as the foundation of modern DNA storage systems. The rationale for using DNA was its extreme theoretical information density of nearly a zettabyte per cm³, its half-life of over 100 years, and its low maintenance and energy requirements. Even slow read and write times did not discourage the idea of using DNA for information storage, as DNA's benefits could be used for cold storage or data security applications. Yet, two important factors limited the development of DNA storage. First, the pressures for extreme scale information storage that exist now, evidenced by Facebook's $1.4 billion dollar, 170,000 m², 11 story tall data center planned for Singapore, were nowhere as exigent 3 decades ago. Second, the cost and scale of sequencing and synthesizing DNA were hard to project being economical, especially before sequencing technologies were accelerated by incentives like the Human Genome Project. However, it is becoming clear that DNA sequencing and synthesis are rapidly dropping in cost. In just 6 short years, the field has transitioned from storing and sequencing back a 0.69 megabyte book in DNA to a 200 megabyte collection of files including a music video. Given these and other trends, and given the maturity and industrially driven nature of much of the research and development in both DNA sequencing and synthesis, it is exciting and reasonable to project that these specific limitations will be surpassed in the near future. For some uses including data security, DNA storage capacities are feasible for practical applications.

The rapid improvements in synthesis and sequencing technologies, and the corresponding increases in sizes of DNA storage systems, is bringing about a metaphorical ‘tipping point’. As system capacities increase, new challenges inherent only to high capacity systems arise. Put simply and intuitively, these challenges derive from the fact that high density and high capacity systems necessarily have many strands in a very small space without any spatial order. One can imagine that in such a situation, it is increasingly difficult to: create a file system with specific addresses for all the content; search for and access only the content that is desired by a user without noise from the very large ‘background’ of other DNA strands; and avoid errors arising from similar but not identical DNA strands physically interacting with each other. To provide some context, the largest 200 megabyte storage system is comprised of roughly 10⁷ unique strands of 150 bp-long DNA, while the solubility of DNA in water is closer to 10¹⁵ strands per microliter volume (˜1 exabyte per microliter). Thus, this drastic difference presents both the physical challenges inherent to high capacity systems, as well as the incredible opportunity to achieve practical extreme scale storage if these challenges can be addressed.

### Synopsis

The following Examples present an analysis of four primary barriers to surmounting this tipping point. The following Examples outline a coordinated suite of experimental and computational capabilities that break down these barriers. The barriers are framed in a thermodynamic context and molecular biology and biochemical technologies are implemented in conjunction with computational design and simulation to both mitigate and harness thermodynamics. In accordance with the presently disclosed subject matter, these Examples fundamentally enable high capacity and high density DNA storage as well as provide useful new functionalities such as file ‘Previewing’, search-in-storage, and metadata retrieval. The following Examples describe results from DNA storage experiments

### Barriers to Practical, High Capacity, and High Density DNA Storage Systems

Barrier 1: High density DNA storage precludes physical partitioning or structuring of DNA strands. A central design question for DNA storage systems is how data will be physically organized. The physical and spatial organization of the DNA strands on a solid surface, on nanoparticles, or even through DNA origami relative to each other could facilitate file access, searching, and indexing. However, any structural features, especially when provided by a scaffolding material, inherently and drastically reduces the storage density of DNA. The reason for this is two-fold. First, even if micron-thin walls were used, for example, to create microwells holding different pools of DNA, the very volume of the walls themselves could have held significant amounts of data in the form of unstructured DNA. The second reason is that even in a 200 megabyte system, there are 10⁷ unique strands of DNA, and it would defeat the purpose of DNA storage to partition these 10⁷ strands into different wells to store just 200 megabytes of data, not to mention the number of wells required to store peta- or exabyte levels of information. Thus, it is an unavoidable challenge for practical DNA storage systems that there will be many unique strands of DNA in close proximity to each other.

Barrier 2: The number of distinct DNA sequence addresses, and total system capacity, is limited by thermodynamics. A resulting corollary of Barrier 1 is that a large number of DNA strands have the potential to interact with each other or with DNA and other molecules that are introduced into the system to access specific content. One's intuition may suggest that as the number of distinct DNA strands increases in a system, it will become increasingly difficult to make sure these strands do not interact non-specifically. For example, if a short oligomer of DNA is used to address a specific strand within a system by hybridizing to it, it will be increasingly likely it could bind a similar but incorrect strand. This concept can be formalized by a thermodynamic framework, as will be described below.

Barrier 3: Lack of techniques to model and experimentally study extreme scale systems. In addition to the two fundamental barriers to extreme scale DNA storage described above, there are also challenges posed by the lack of research infrastructure capable of studying extreme scale systems. This is in part because DNA synthesis technologies are not yet advanced enough for de novo synthesis of gigabyte and higher levels of DNA at reasonable costs for research groups. In addition, many of the computational tools and simulations have not been adequately developed due to a lack of experimental data that can inform them. Likewise, without computational tools, it is difficult to design experiments to efficiently test hypotheses about extreme scale systems. What are needed are methods to mimic the properties and behaviors of extreme scale systems through a combination of simulations and high throughput experimental approaches.

Barrier 4: High capacity DNA storage will preclude the ability to sequence an entire library in a cost-effective or efficient way, even as sequencing costs drop and speeds increase. For archival storage, there will be no digital copy available for faster access. Finding the desired data in a large archive may be dependent on a costly and serialized sweep of an entire library. Strategies are needed for mitigating the relatively limited sequencing bandwidth, for example molecular processing-instorage or techniques to make better use of sequencing bandwidth. Intriguingly, in the context of processing-in-storage, selectively increasing the likelihood of non-specific hybridization may yield benefits for more efficient searching of data within storage.

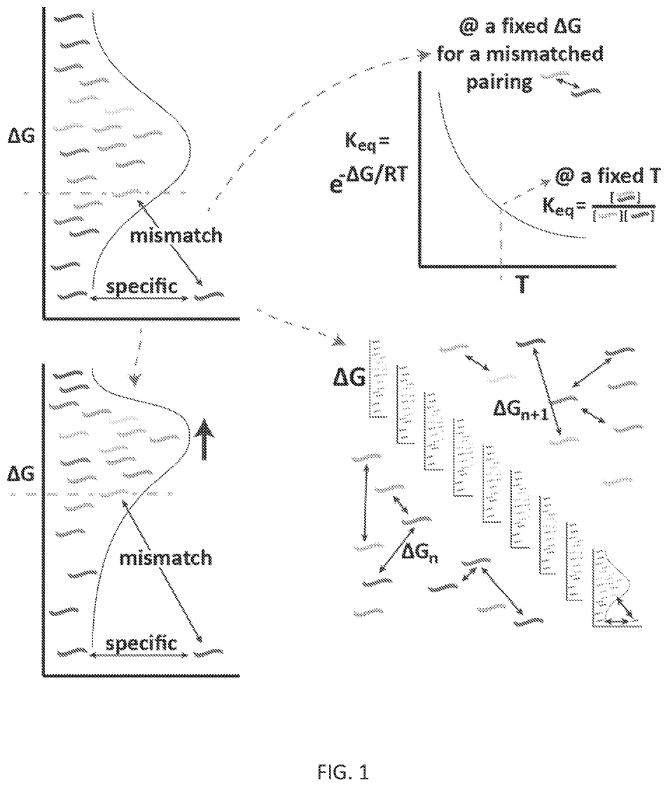

It is useful to conceptualize the barriers to DNA storage within a statistical thermodynamic framework (FIG. 1), as it is the field of science describing the configurations of molecules within systems based upon differences in the energetics of molecular interactions and entropy. FIG. 1 (top, left) depicts a collection of different DNA strands, with strands having similar DNA sequences matching closer in gray scale to each other. One way to depict the likelihood two strands will bind to each other based upon DNA base pairing complementarity (A binding to T or C binding to G) is through a calculation of the Gibbs free energy of binding (ΔG). The greater (or less negative) ΔG is, the less likely two strands are to bind (‘mismatch’). Within this thermodynamic framework, strategies to achieve desired goals, engineer specific data manipulations, and create overall effective architectures for DNA storage systems are provided through the following approaches.

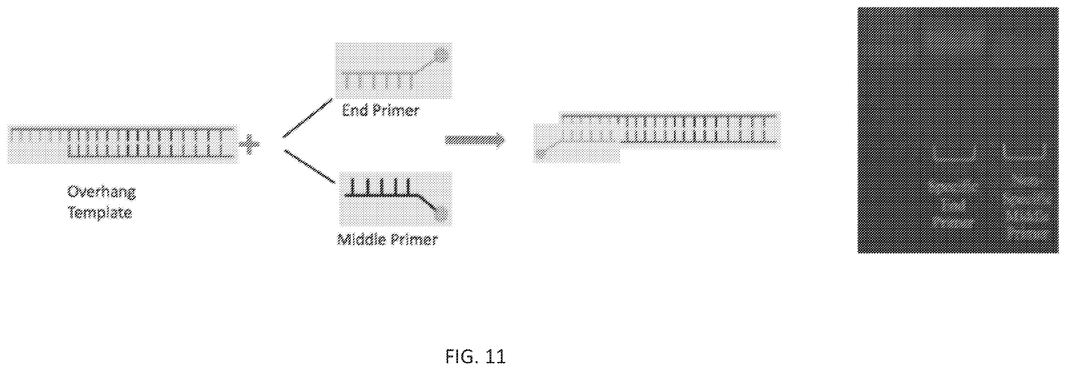

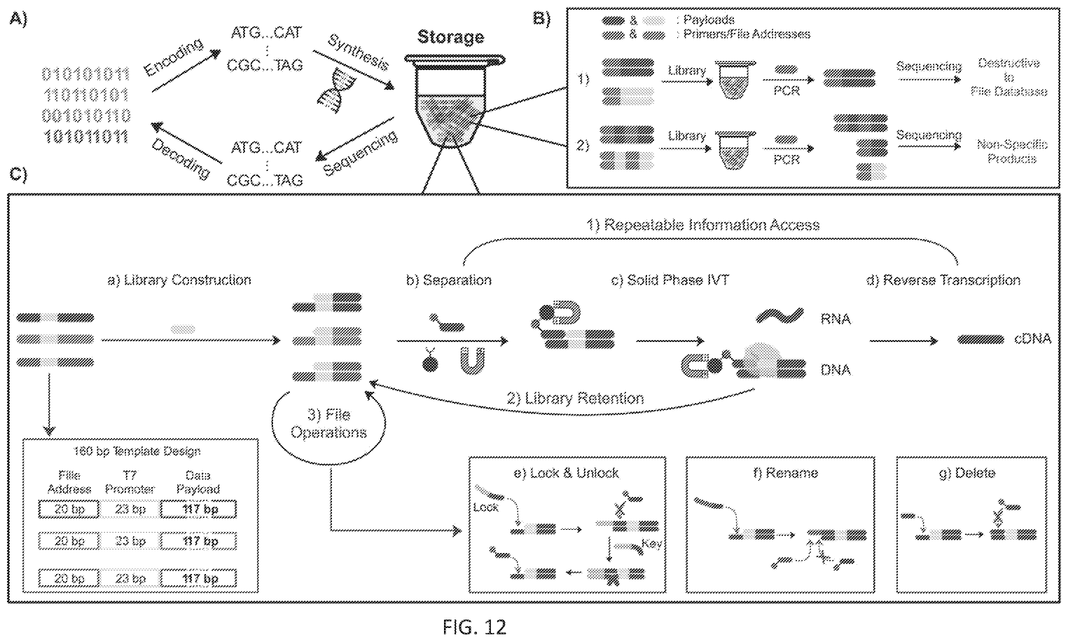

In accordance with the presently disclosed subject matter as set for in the following non-limiting Examples, temperature and concentration are used as control knobs to implement novel processing in storage such as “Preview”, keyword-tag retrieval, and metadata functions. The concept here is to take advantage of the fact that some DNA strands are more similar to each other than others, and that these ‘mismatch’ interactions can be harnessed for a range of uses. For example, for two particular mismatched strands (FIG. 1, shown by arrows), by changing either the concentration or temperature, one should be able to control the amount of mismatch binding. This can be used for ‘previewing’ a file at one temperature or concentration, while accessing the full file at another temperature or concentration. A collection of binary mixtures of DNA strands were created where a 60 bp strand had a 4 Hamming Distance difference from the primer, and a 200 bp strand had perfect complementarity to the primer. PCR was performed at high (60 C) or low (45 C) temperatures or at high (500 nM) or low (125 nM) primer concentrations. The amplification of the incorrect or correct strands were able to be distinguished on a Fragment Analyzer based upon their different sizes. This procedure identified primers and sequences that differentially amplified strands based upon temperature or primer concentration. See also FIGS. 10A and 10B.

In accordance with the presently disclosed subject matter as set forth in the following non-limiting Examples, non-specific random file access is reduced and address space is increased by physical occlusion of non-specific binding sites. In a more standard scenario, mismatches would typically not be desired at all as they would lead to errors in accessing information. Restricting all DNA strands to have very different sequences to one another is one approach but would be detrimental to the capacity of the system. Instead, a more elegant solution maintains the same sequence space used, but shift the energetics of binding so that mismatches have greater ΔG (FIG. 1, bottom left). In accordance with the presently disclosed subject matter as set for in the following non-limiting Examples, this is achieved through physical occlusion of most of the DNA in the system except for the addresses (sequences used to access information or files).

In accordance with the presently disclosed subject matter as set for in the following non-limiting Examples, the maximum number of addresses possible is computationally and experimentally estimated. This approach addresses a longstanding, unanswered question in the biological sciences, systems biology, and DNA storage: how many unique addresses are possible in a storage system? This question strikes at the heart of understanding the capacity limits of storage systems. In accordance with the presently disclosed subject matter as set for in the following non-limiting Examples, computational and high throughput experimental approaches are provided to more generally understand molecular DNA interactions and to determine the design rules relating DNA sequence to ΔG.

In accordance with the presently disclosed subject matter as set for in the following non-limiting Examples, searches are performed in storage by engineering binding interactions amongst DNA strands. With no electronic record as backup, searching a DNA library can be expensive and time consuming, requiring many files to be sequenced to find the one that is desired. By designing a short oligo that hybridizes to the sought-after data, that oligo can act as a query in solution, and the hybridized strands can be extracted as an answer to the query. Computational tools are designed to construct such a thermodynamically conducive encoding and query that hybridizes, and are experimentally verified in a DNA-storage system in accordance with the presently disclosed subject matter.

### Overview of Examples 1-6

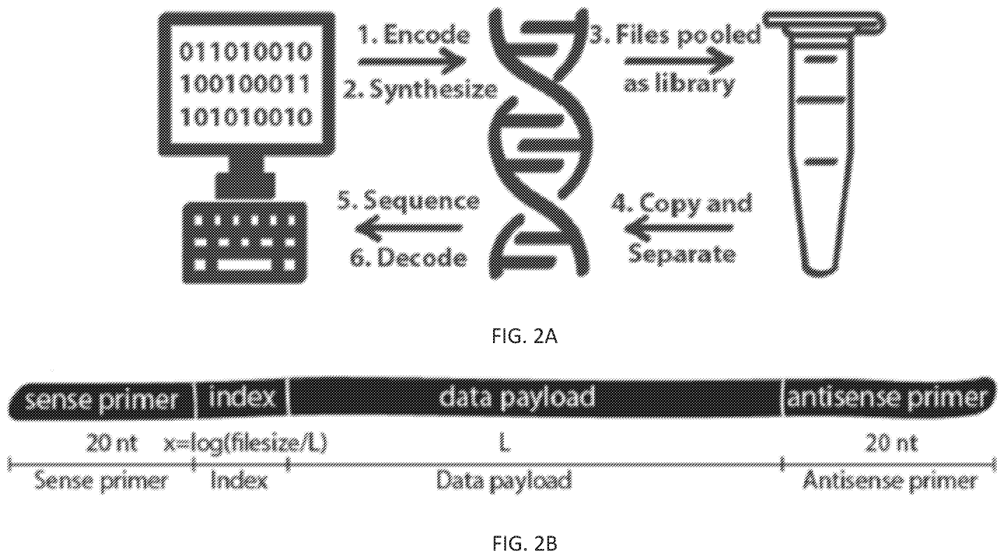

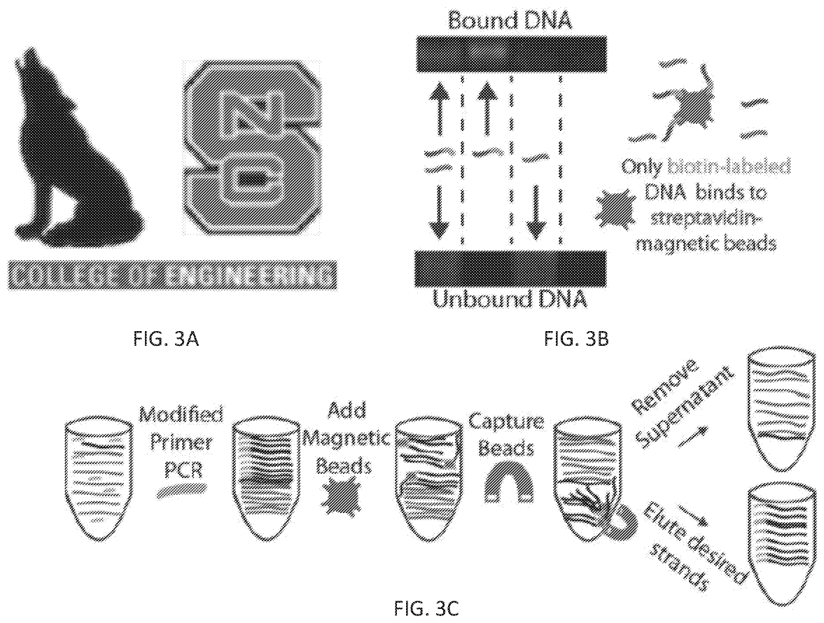

Examples 1-6 relate in some aspects to extreme-scale DNA-based storage systems. These Examples analyze the limitations of current state-of-the-art DNA storage systems. The general framework for such systems is shown in FIG. 2A: 1. Digital data is converted into DNA sequences through a choice of encoding schemes. 2. The DNA is chemically synthesized. 3. All of the different DNA strands are mixed into one pool in a solvent and stored in a test tube. 4. To read the data, many copies of the DNA strands are generated by polymerase chain reaction (PCR) amplification. A particular file can be read by using PCR primers that amplify a specific set of DNA strands. The remainder of the strands become lower in abundance relative to the desired file's strands. 5. The DNA is then read by next generation sequencing (e.g. Illumina or Oxford Nanopore sequencing). 6. the DNA sequences obtained are decoded. The architecture of the DNA strands is shown in FIG. 2B and comprises a pair of primer binding sites that are used to amplify specific data. An index sequence allows the order of many strands comprising a file to be deduced, while the data payload comprises the remainder of each strand.

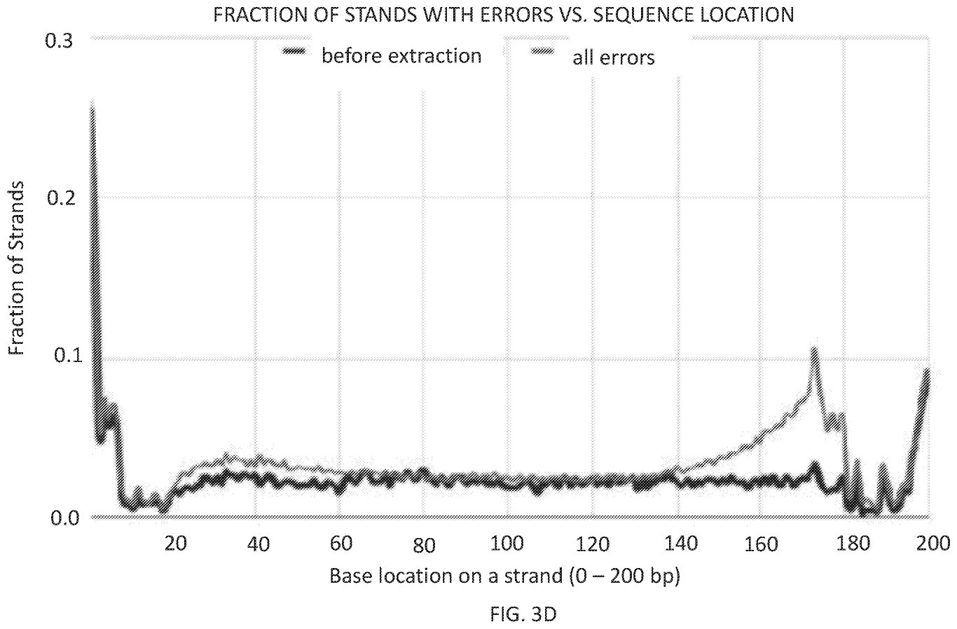

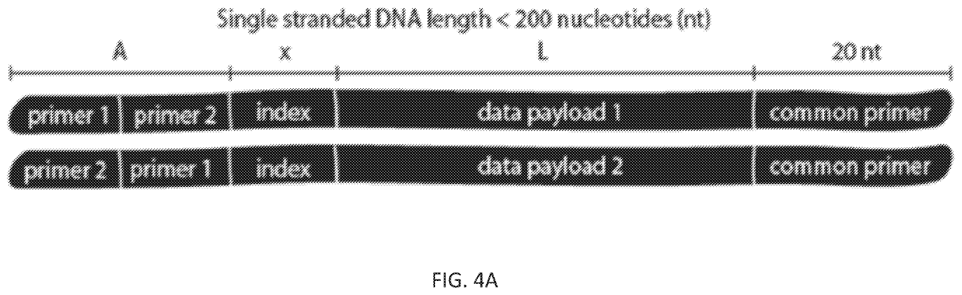

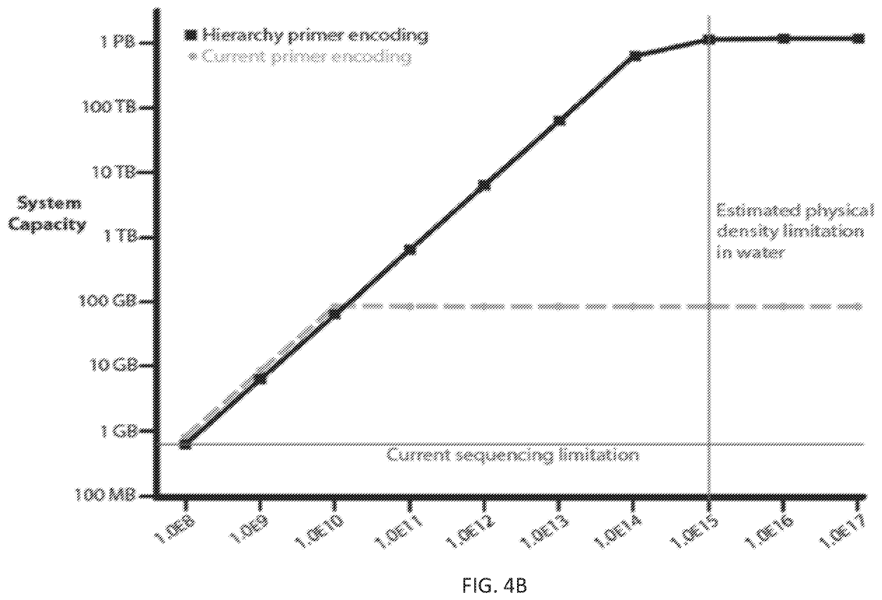

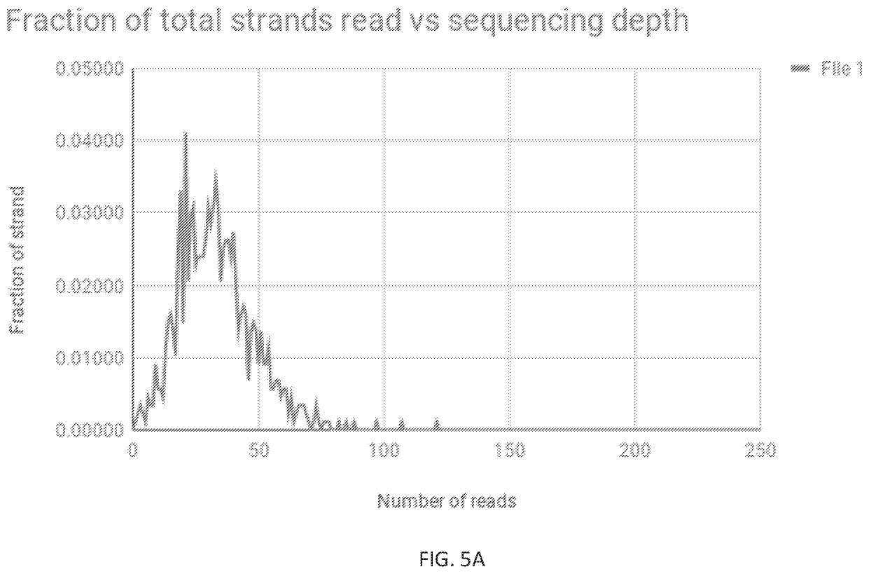

Most of the work in the art has focused on improving steps 1 & 6 (encoding strategies), and steps 2 & 5 (DNA synthesis and sequencing technologies). However, when envisioning the physical nature of a truly extreme scale system (steps 3 & 4), clear challenges arise that require a combination of simulations and molecular technologies to solve. To explore and address these challenges, a pool/library of DNA comprising 6000 unique DNA strands (FIG. 3A) was prepared. This library comprised 5 files, the Declaration of Independence, three image files of NC State University logos, and the Bill of Rights.

### Example 1

### Extraction and Error Analysis of Individual Files from a Database

At high capacity, there will be many DNA strands. This poses numerous challenges. First and foremost, current approaches rely on exponential PCR amplification of a desired file to overwhelm the rest of the DNA library/database. However, at extreme scales, a file will comprise such a small percentage of the total database that even after PCR, the background noise of the library can be overwhelming (library will be ˜10¹²-10¹⁵ strands/μL, while PCR typically amplifies target DNA to <10¹² strands/μL). This also means background data of the database. (The highest end next generation sequencing capabilities are able to read ˜10⁸ strands total, inclusive of redundant copies of strands.) Furthermore, given sequencing limitations, all desired strands may not even be able to be sequenced since so much of the sequencing space is taken up by background strands. To address this problem, an extraction scheme was implemented (FIG. 3C), where a chemical moiety is linked to a primer used to amplify DNA strands of a specific 1000-strand file. A magnetic bead is functionalized with a corresponding chemical group that binds the primer moiety, enabling the specific file to be extracted using a magnet. It has been demonstrated that 4 distinct systems are capable of this form of efficient file extraction (biotin-streptavidin, fluorescein antibody, digoxigenin-antibody, polyA-polyT oligomers) (Table 3). Next generation sequencing results of the extracted file (‘elution’) and the rest of the retained library that was not extracted (‘supernatant’) show that this method is able to greatly enrich for the desired file (to >90%) while retaining all files within the original library so it can be reused (Table 4). It has also been demonstrated that the same 1000-strand file can be repeatedly extracted from the same 6000-strand library (i.e. this process is nondestructive). Additionally, it has been shown that error analysis of the next generation sequencing data can be performed to infer where at what frequency errors in the process are occurring. This plays a role in benchmarking how robust encoding strategies and algorithms need to be.

TABLE 3
Superior Capability of 4 Extraction Methods Compared to PCR Alone
Fraction of Total Strands ~3%:97% ratio

| Sample Name | File 3 | Background |
|---|---|---|
| Random Access - 30 cycles | 23% | 77% |
| 6 cycle unmodified control | 1% | 99% |
| Biotin elution | 58% | 42% |
| Biotin supernatant | 1% | 99% |
| Fluorescein elution | 50% | 50% |
| Fluorescein supernatant | 1% | 99% |
| Digoxigenin elution | 92% | 8% |
| Digoxigenin supernatant | 0% | 100% |
| Poly(A)-25 elution | 100% | 0% |
| Poly(A)-25 supernatant | 2% | 98% |

TABLE 4
Sequencing Data Demonstrates the Successful Separation
and Enrichment of 3 Different Files

| Sample | File 1 | File 2 | File 3 | File 4 | File 5 |
|---|---|---|---|---|---|
| Library | 11% | 19% | 17% | 33% | 26% |
| File 1 supernatant | 25% | 15% | 21% | 24% | 15% |
| File 1 elution | 89% | 0% | 2% | 0% | 9% |
| File 2 supernatant | 10% | 29% | 16% | 25% | 20% |
| File 2 elution | 1% | 90% | 1% | 6% | 1% |
| File 3 supernatant | 8% | 8% | 28% | 26% | 29% |
| File 3 elution | 1% | 0% | 91% | 6% | 2% |

### Example 2

### Extraction Methods are Useful as Library Capacity/Strand Number Increases

Because sequencing space is limited, and it is also economically prudent to sequence only files that are desired, the advantage of the extraction method was directly demonstrated over standard PCR approaches (Table 1). All extraction methods reduced the background data that was sequenced compared to PCR, even in cases where the file comprises a small fraction (3%) of the total database. Due to financial constraints of ordering very large databases of DNA strands, the library size was only 6000 unique strands. Many copies of the same strands were extracted. However, it can be projected that if all strands were unique, this and other experiments that were performed demonstrate the extraction of 1 gigabyte of data from 10 terabytes of background database data. Thus, with the capability to order higher diversity DNA libraries, this system can already handle common electronic file sizes.

### Example 3

### Hierarchical Encoding with Extraction Technologies Solve Current System Capacity Limits

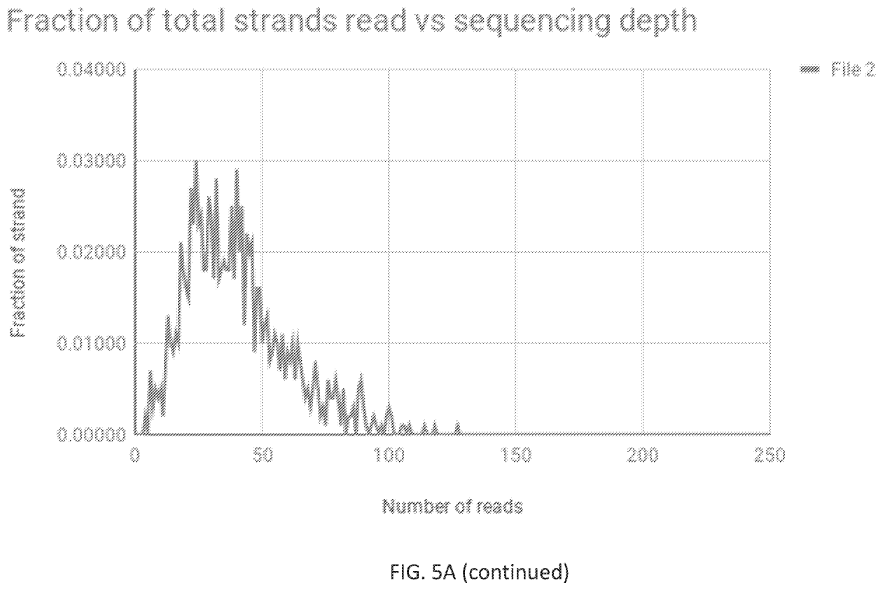

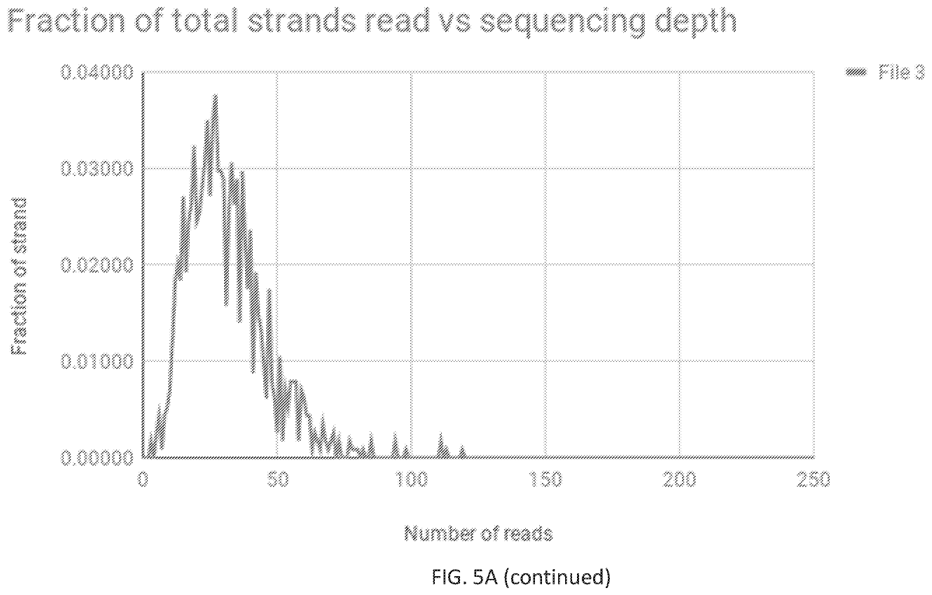

As the number of strands in a database increases, another challenge that arises is that the number of primers available to specify distinct files becomes limiting. This is because even though there are theoretically 4²⁰ distinct 20 bp-long primers possible, most of these will be similar enough that they will interact non-specifically with each others' binding sites. The length of primers is also constrained by thermodynamics and the temperature at which the storage system will reasonably operate (between room temperature and 100° C.). It has been estimated that there may only be <20,000 usable primers. Assuming an average file size of ˜6 MB, this limitation results in a limit on total system capacity of ˜100 GB (FIGS. 4A and 4B). To break this limitation, a hierarchical encoding scheme was implemented where primers are nested. This more than exponentially increases the number of unique ‘addresses’ for files, without increasing the total number of unique primers needed. Nesting 2 primers would enable 1 PB capacities. Nesting even more than 2 primers would result in even larger numbers of total addresses (i.e. 20,000 unique primers^{N number of nests}). This strand architecture was combined with the extraction methodology of Examples 1 and 2 and successful extraction decoding of two different files (one encoded by primer 1 followed by primer 2, the other primer 2 followed by primer 1) was demonstrated. The desired file in each case comprised over >90% of the sequencing data, showing the specificity of hierarchical addresses.

### Example 4

### Error and Noise Analysis Demonstrates an Advantage of Extraction Strategies Over PCR

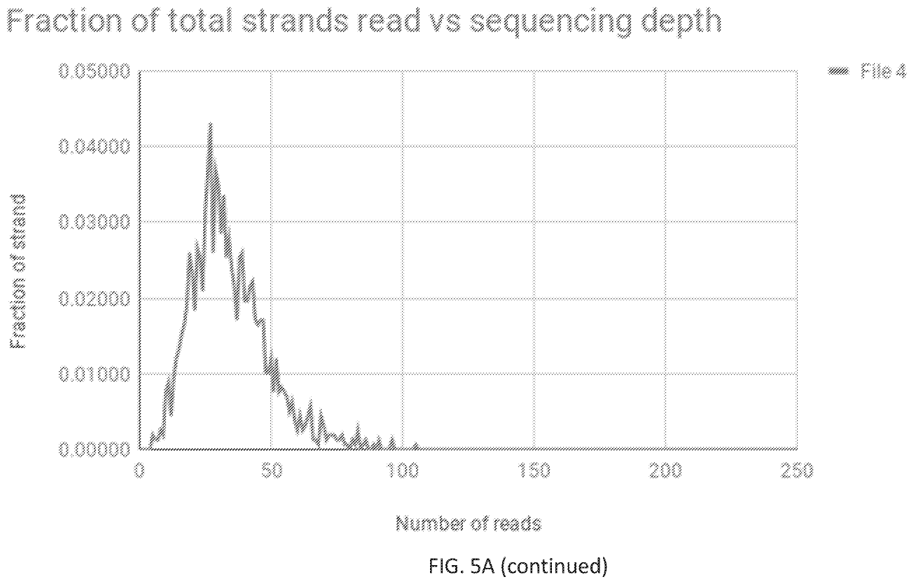

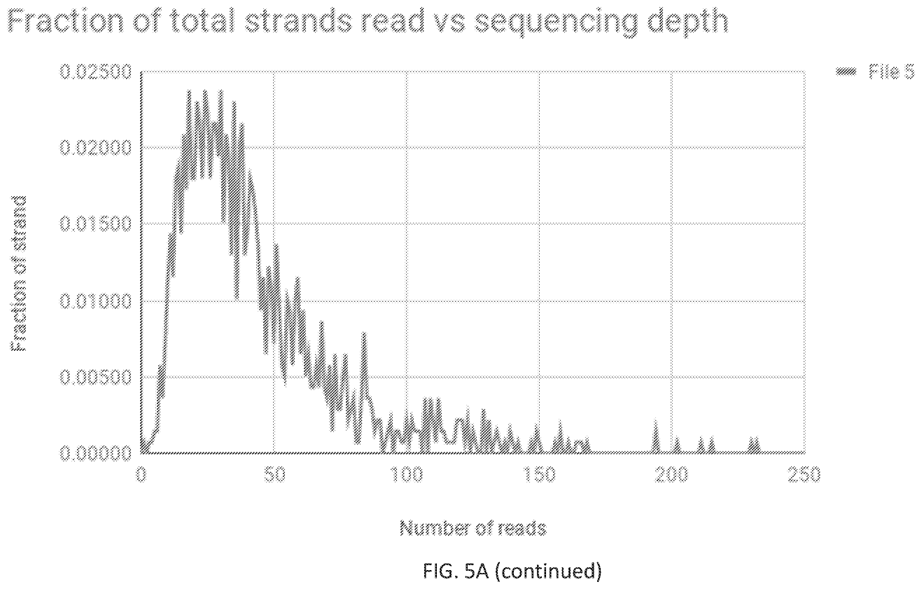

With next generation sequencing it was demonstrated that the distribution of strands of a file (FIG. 5A, left) increases with PCR cycles and remains high (FIG. 5B, right, top line). A broader distribution of strands per file means that more copies of a file need to be sequenced to make sure at least one copy of each of the 1000 unique strands is captured in sequencing.

### Example 5

### Harnessing Non-Specific Interactions for Useful In-Storage Functions

While ostensibly undesirable, it may be useful to intentionally enable interactions between mismatched DNA strands to occur, especially if the interaction can be switchably controlled. Based upon a simple thermodynamic analysis (FIG. 1), lowering the system temperature or increasing the concentration of primer should increase the equilibrium binding of a primer to a mismatched binding site on a DNA strand or file. This was demonstrated by mixing a primer with both a 150 bp-long DNA strand containing a perfect binding site match (FIG. 6A, top bands) and a DNA strand containing a mismatched binding site (FIG. 6A, bottom bands). PCR of the mixture was performed at either 60° C. or 43.5° C. and it was found that the PCR product formed (i.e. ‘file’ or strand ‘accessed’) could be shifted from the perfect match to mismatch strands simply by lowering the temperature. Similarly, increasing primer concentration allowed the mismatch strand to be amplified (FIG. 6A, bottom).

### Example 6

### Physical Occlusion of Data Payload Regions Prevents Errors in File Access

Mismatch interactions could also occur between primers and the middle or payload regions of DNA strands. This in fact could be a more pressing issue as the data payload regions contain a much larger sequence space. Current approaches to simply select primers with sequences that are very different from all data payload sequences would place a significant restriction on the number of primers available to use, and a corresponding limitation on overall system capacity. To address this issue, the use of PCR was avoided, where double stranded DNA (dsDNA) is melted into single stranded DNA (ssDNA) in each cycle as this allows primers physical access to all of the sequence space. Instead, DNA strands comprised primarily of dsDNA were created but with a 20 bp ssDNA overhang (FIG. 6B). Without using PCR, it was shown that a biotin-modified primer that binds to the exposed ssDNA overhang is able to extract the DNA strand through interaction with a streptavidin-functionalized magnetic bead. In contrast, a biotin-modified primer complementary to a sequence in the dsDNA region was not able to bind and extract the DNA strand.

### Summary of Examples 1-6

Examples 1-6 show the extraction of a specific file from a larger DNA-encoded information database. Examples 1-6 demonstrated ability to access and analyze data from an actual 5-file, 6000-strand DNA database; physically mimicked accessing a 1 GB file from a 10 TB database; modeled and implemented hierarchical strand encoding to increase capacity by 10⁵; demonstrated, with a small number (1-3) of strands, that temperature and concentration can be used to intentionally control primer binding to off-target or mismatch DNA strands; demonstrated, with a small number (1-3) of strands, that a hybrid ssDNA and dsDNA strand structure can block primers binding to off-target or mismatch DNA sequences in the data payload region; demonstrated that direct primer hybridization to a ssDNA overhang can abrogate the need for PCR in accessing or extracting a file.

### Overview of Examples 7-9

Examples 7-9 specifically address challenges arising from the physically ‘crowded’ and diverse nature of high capacity systems. Despite the fact that DNA synthesis technologies are not yet economical enough to synthesize GB and higher systems, creative approaches are provided that can either mimic or truly create GB through PB level systems. This approach and focus on capacity limitations plays a role in provided practical DNA storage systems. Examples 7-9 relate to temperature and concentration as control knobs to implement novel processing instorage such as “Preview”, keyword-tag retrieval, and metadata functions.

In Examples 1-4, it was demonstrated that temperature and concentration control the binding of PCR primers to mismatched sequences. This phenomenon can be useful in implementing in-storage functions such as “Previewing” a file, keyword or tag retrieval, or reading metadata. Example 7 relates to a predictive model to design PCR primers that can exploit temperature and concentration differences. Example 8 relates to an experimental strategy to screen millions of combinations of primers and mismatched binding sites. This both identifies real primer sequences that can be used in practical storage systems, but also inform the model generated in Example 7. Finally, in Example 9 the model and tools are harnessed to implement a 10 KB “Preview” of a 10 MB file.

### Example 7

Thermodynamically Informed Model and Simulation to Design and Predict Putative Primers with Differential Binding across Temperature and Concentration Gradients

There are several methods to predict the likelihood of primers binding to mismatched sequences. These include Hamming Distance (number of mismatched base pairs in a linear position-by-position comparison), Edit Distance (number of transformations that include deletions, additions, mutations to convert one sequence to another), and binding energy calculations such as Gibbs free energy.

Three models are created, one based upon Hamming Distance, one on Edit Distance, and one based purely on Gibbs free energy. These are coded to be as computationally inexpensive as possible to allow exploring a large set of primers. (The actual calculation of Gibbs free energy is difficult due to the many molecular conformations and hairpin structures that even short DNA strands can adopt. Fortunately, many open source tools have already been developed to perform thermodynamic analysis of short oligos, such as NUPACK (Zadeh et al., (2011) J. Comput. Chem. 32, 170-173), Primer3 (Untergasser A, et al., (2012) Nucleic Acids Res. 40(15):e115-e115), and Oligo Calc (Kibbe W A. (2007) Nucleic Acids Res. 35(webserver issue): May 25). The existing algorithm is built upon to first screen out ‘bad’ primers that have properties falling outside of traditional specification bounds used in molecular biology (balanced GC content, melting temperature within a predetermined range of 50 to 60° C., no hairpins or high likelihood of self-hybridization). Using this model, a set of 1,000 primers with distinct 20 bp sequences are designed to be very different and highly unlikely to bind to each other and each others' target sequences (if possible as the model may suggest fewer than 1,000 primers are available). In addition, for each primer, 5,000 variants with different Hamming and Edit distances are created. In a represensentative, non-limiting approach, this entire collection of 5 million primers is ordered from a commercial source, such as Twist Biosciences, and tested for their ability to bind each others' target sequences in different primer concentrations and temperatures in a high throughput, single-pot experiment described in Example 8.

Using the results from Example 8, thermodynamically inspired heuristics against the observed experimental results are prepared. The heuristics can be tuned based on the findings. However, machine learning techniques are employed as needed to help infer a more predictive model that relates the computed properties against the experimental results. Linear classifiers may be sufficient for this task, since it is only needed to predict. Using this model, a new set of 1,000 primers is predicted that are not expected to interact, and an additional 5,000 variants of each primer. This pool of primers is tested experimentally, compared against the models' predictions of binding, and further iterated as necessary.

### Example 8

### High Throughput Screen to Identify Design Rules for Concentration, Temperature, and Primer Sequence Space

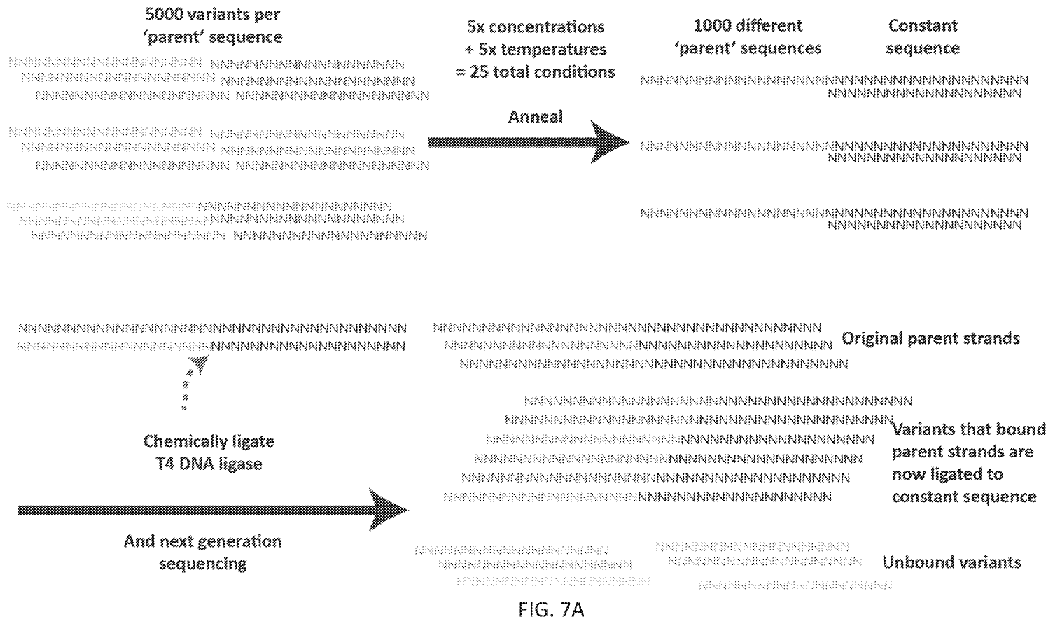

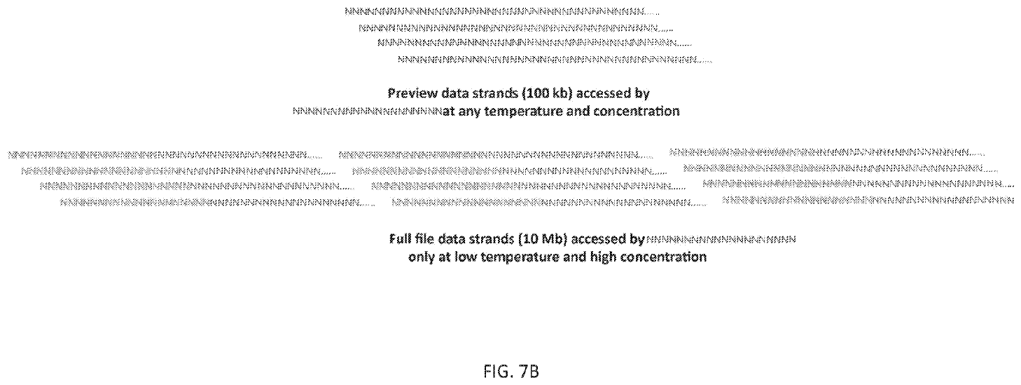

In conjunction with Example 7, a high throughput approach is provided to measure the interactions between millions of primers as a function of primer concentrations and temperature. Next generation sequencing's ability to measure ˜300 million distinct strands of 150 bp-long DNA is employed. To do this, primer-binding events are recorded into the sequence of DNA. To do this, the strategy orders the 1,000 20 bp-long ‘parent’ primers designed in Example 7 appended to a constant 20 bp DNA sequence, to create an overall 40 bp-long ssDNA (FIG. 7A, middle). An equal concentration of a primer complementary to the constant 20 bp sequence (black) is added to create a DNA strand that is half dsDNA and half ssDNA. The additional 5 million ‘variant’ primers designed in Example 7, are then added at 0.01, 0.1, 1, 10, and 100 fold differences to the parent primers' concentrations. The mixture is incubated at a range of temperatures: 40° C., 45° C., 50° C., 55° C., 60° C.

Variant primers that bind to the ssDNA parent primer overhang sequences complete a fully double stranded 40 bp-long DNA strand, and a DNA ligase enzyme is added to covalently link the bound variant primer to the adjacent constant primer. This ‘locks’ in the primer binding event into the sequence of the DNA. Any unbound ssDNA overhang parent primer DNA and variant primers, but not dsDNA, are enzymatically degraded by addition of Mung Bean Nuclease. The resultant mixture is comprised only of 40 bp-long dsDNA and some leftover 20 bp-long dsDNA which is just the constant primer sequence. To maximize use of sequencing space, this mixture of DNA strands is randomly ligated to each other using blunt end ligation until strands approximately 160 bp-long are obtained. There are 5 temperature and 5 primer concentrations tested (25 total samples). Each sample is prepared for next generation sequencing using Illumina barcodes appended to all strands within a sample. This allows all samples to be sequenced in one ‘lane’ of a sequencing run. Variant primers that are enriched by temperature or primer conditions are chosen for further validation, in isolation of other variants.

### Example 9

### 100 KB “Preview” of a 10 MB File

Variant primers with the best temperature- and concentration-dependent differential PCR amplifications in Example 8 are selected for a scaled-up implementation of a common function associated with personal computing: ‘Preview’. Preview provides a low-resolution image or one-page preview of a file so the user can identify if it is a file he or she wishes to open. A 10 MB video file is encoded into approximately 2 million 200 bp-long DNA strands (FIG. 7B). The majority of the strands are flanked by 20 bp sequences that bind with mismatches to the primer. A subset of the strands will have perfect 20 bp complementarity to the primer. This subset of strands encode a 100 KB low resolution sequence of 4 Preview screenshot images from the video. Two PCRs are performed: one at high temperature and low primer concentration which should access only the 10 KB of preview strands; the other at low temperature and high primer concentration which should access all strands of the entire 10 MB video file.

Alternative Approaches for Examples 7-9. If excessive non-specific primer binding is observed, it is difficult to identify suitable primers for temperature and concentration swing. This is unlikely in a high throughput scenario, ˜10 primers that work using a low-throughput screen are identified. However, if this occurs, discovering this through a high throughput screen is useful information for the research community. The quantification of the extent of non-specific interactions would be useful and further suggest the need for temperature and concentration based manipulation of DNA systems to augment storage capabilities. If the models initially have difficulty predicting the experimental results, two approaches are employed: 1) primer designs are made more stringent and distinct from each other in order to create a set of practical primers, even if this means losing a significant portion of potentially suitable primers; 2) the opposite approach is taken and machine learning is implemented with an order of magnitude larger experimental data sets.

### Overview of Examples 10-12

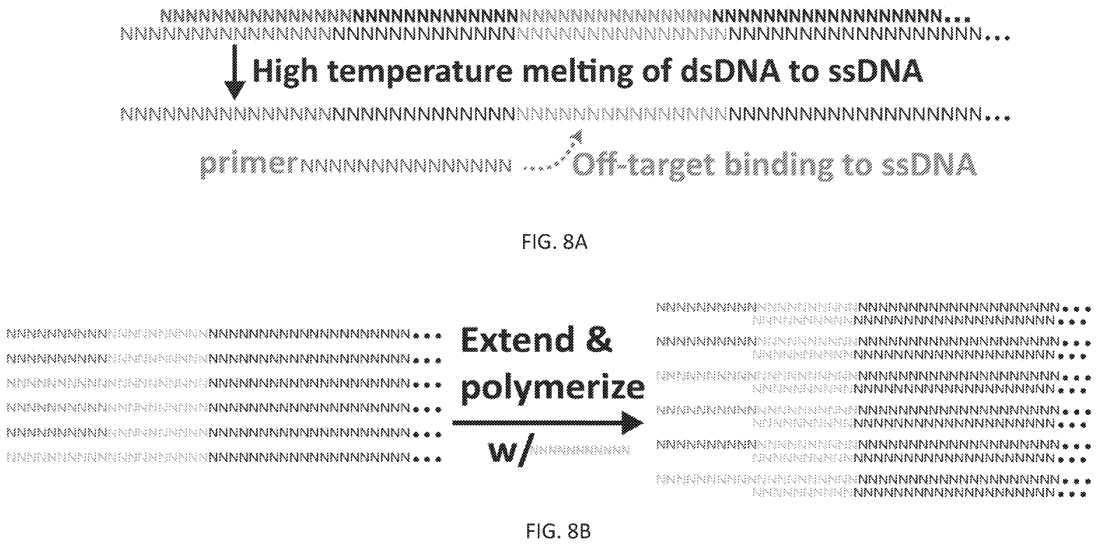

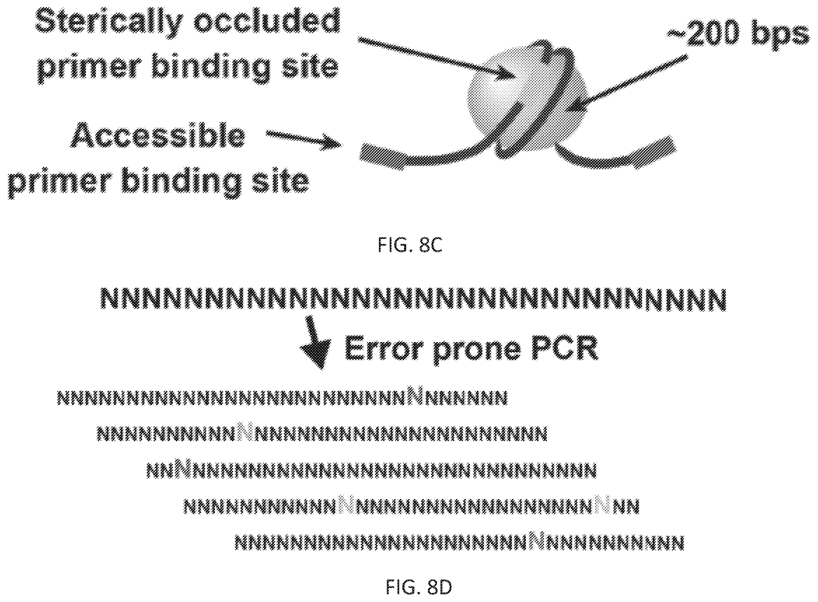

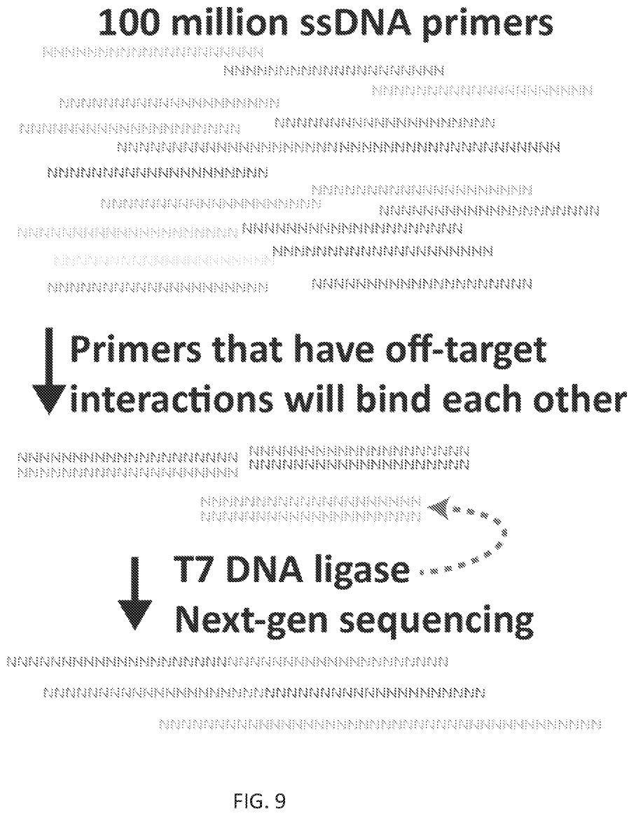

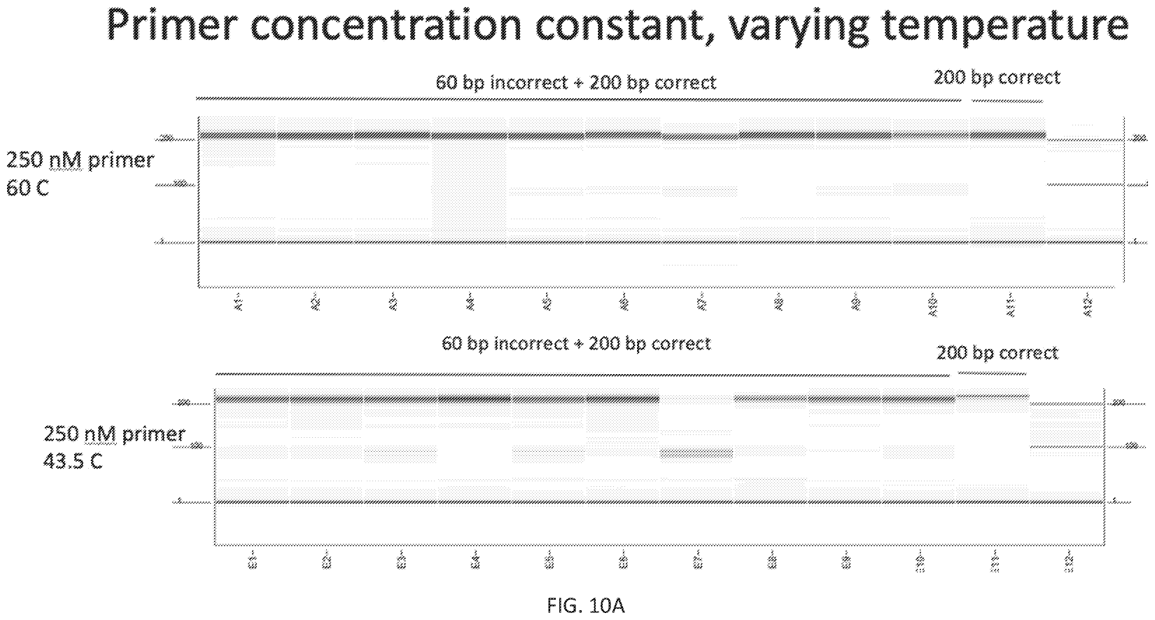

Examples 10-12 reduce non-specific random file access and increase address space by physical occlusion of non-specific binding sites. In general, in high capacity systems there are many potential mismatch interactions that are undesired, even at a single operating temperature and primer concentration. A related concern is that in a PCR reaction, dsDNA is ‘melted’ into ssDNA in a high temperature step (typically >90° C.) (FIG. 8A). This allows a primer to bind the ssDNA and begin polymerization. However, this process also means that primers could bind non-specifically in the middle of DNA strands, including in the payload region, not just to other primer binding sites. In other words, off-target interactions with other primer binding sites is actually a very small fraction of the potential off-target interactions possible in a high capacity, PCR-based storage system. A goal of Examples 10-12 is to develop physical methods to block primers from ever binding data payload regions; in other words, the free energy of a primer binding is increased to any region except the primer binding regions of DNA strands (FIG. 1).

### Example 10

### ssDNA-dsDNA Hybrids to Block Erroneous File Access

In Examples 1-6, it was demonstrated (FIG. 6B) that dsDNA with single stranded ‘overhangs’ are able to block primers from binding to the payload region of DNA strands. This approach is first scaled to a KB-sized file to test that this ‘direct pullout’ will work on a file comprised of thousands of distinct DNA strands. A library of diverse dsDNA strands that have a 20 bp ssDNA overhang is efficiently created. This form of DNA cannot be ordered from DNA synthesis companies. Previously, two complementary ssDNAs, one being 20 bp longer than the other were ordered, mixed together, heated the sample to 98° C., and gradually lowered the temperature until the two ssDNAs annealed together. This approach could pose difficulties in a scaled-up system, as many diverse ssDNAs need to be annealed together in a single ‘pot’ or test tube. It would be infeasible to individually anneal each pair of ssDNAs in their own test tube when storage systems scale to 10¹² or more distinct DNA strands. Therefore, a method is designed in which the longer 200 bp ssDNAs are chemically synthesized (such as by ordering from a commercial source, such as Twist Biosciences) where every ssDNA has the same primer binding site located 20 bp inset from its end (FIG. 8B). This primer site is designed to be very different than all other sequences that would show up in the data payload regions. A simple way to do this is to ensure multiple dinucleotide repeats be in the primer, as repeated nucleotides are often avoided in data payload regions through rotating encodings to minimize issues with secondary DNA structures forming or causing issues with downstream DNA sequencing. This primer is then added to the mixture of ssDNAs and several cycles of PCR are carried out to create a library of dsDNA strands with 20 bp ssDNA overhangs. This library is purified using a silica column to remove free primers and ssDNAs, and the size of the remaining strands is confirmed to be between 180 and 200 bp using, for example, a Fragment Analyzer machine (Advanced Analytical, Inc.). 10 files of 10 KB each are encoded into the library, with each file having a unique ssDNA overhang. Biotin-labeled primers are used to pull out each file from the database, and next generation sequencing is performed to confirm specific access of only the desired files of interest.

### Example 11

### Nucleosome-Based Occlusion to Block Erroneous File Access

Example 10 creates libraries of dsDNA strands with 20 bp ssDNA overhangs. However, an alternative approach is pursued that does not require this unique strand structure and instead use dsDNA alone. Inspiration is drawn from the natural structure of genomic DNA in eukaryotic cells (nucleus-containing cells including those of yeast, plants, and mammals). Eukaryotic DNA is actually ubiquitously complexed with octamers of histone proteins to create units known as nucleosomes (FIG. 8C). ˜150 bps of dsDNA wraps around each protein octamer in a nucleosome. Interestingly, it has been reproducibly reported that nucleosomes occlude the ability of DNA-binding proteins to access and bind their target DNA sequences. This property of nucleosomes is harnessed to block mismatch interactions in data payload regions of DNA strands. A library of 200 bp dsDNAs ordered and assembled into nucleosomes using previously published protocols (Mattirioli et al., (2018) Bio-protocol 8(3): e2714). Nucleosome assembly is confirmed by native western blots and fluorescent labeling of DNA on polyacrylamide gels. Controls include the 601 Widom sequence complexed to nucleosomes, which can be ordered from multiple companies (e.g., ActiveMotif, Inc.). To access files, the exposed ends of the 200 bp dsDNA strands include 20 bp address sequences (ends are not occluded by the nucleosome, FIG. 8C). These sequences can be bound by a protein called dCas9, provided a complementary ‘guide RNA’ is also provided. This system, known as CRISPR, is especially useful because the dCas9 protein can be retargeted simply by providing a guide RNA with a complementary sequence to the target DNA. The dCas9 protein is purchased linked to biotin. Specific files are pulled out of the DNA library using a streptavidin-conjugated magnetic bead.

### Example 12

### True Extreme Capacity System of 1 Petabyte Using an Error-Prone Generated Background Library

With either one or both of the two systems developed in Example 10 and 11, they are challenged to function in a truly extreme capacity system. One of the major challenges with studying extreme scale systems, and a reason they essentially have not been studied in the context of DNA storage, is that they cannot be chemically synthesized with modern DNA synthesis technologies. The largest system to date (Organick et al., Nature Biotechnology volume 36, pages 242-248 (2018)) was comprised of 12 million distinct 150 bp-long strands of DNA and required the resources beyond that available to a standard academic research group. While it is widely anticipated that DNA synthesis costs will drop dramatically in the coming decade (Carlson, R., Nature Biotechnology volume 27, pages 1091-1094 (2009)), physical approaches are needed to study extreme scale systems before the synthesis capabilities actually arrive. This Example involves borrowing a method from the molecular biology and protein engineering fields called Directed Evolution, in which a sequence of DNA encoding a protein is randomly mutagenized in a PCR reaction through the use of an ‘error prone’ polymerase enzyme or error prone nucleotide analogues. In each cycle of an error prone PCR, random mutations to the DNA sequence are introduced (FIG. 8D). It has been demonstrated that just one initial 500 bp DNA sequence can be converted into a library with a diversity of 108 different sequences. A 1 petabyte library (˜10¹⁵ distinct DNA strands of 200 bp length) through error prone PCR by starting with a chemically synthesized library of 10⁶ unique DNA strands and randomly mutagenizing the library. Because DNA sequencing is not able to read 10¹⁵ strands (currently it can only sequence about 3 GB per run), Random samplings of the PB-scale library are taken and the system is modeled with a binomial distribution to estimate the true diversity and information capacity of the system. The strands of this library are randomly generated so it is not be able to encode actual files or data. However, this library is used to physically simulate a true 1 PB scale DNA storage system by dosing in smaller files on the order of KB to MB in size and asking whether those files are accessed using the systems in Examples 10 and 11.

Alternative Approaches for Examples 10-12. A ‘physical occlusion’ system to inhibit nonspecific access of data is obtained, particularly in light of a strong demonstration of a successful ssDNA overhang system herein above. Furthermore, Examples 10 and 11 are alternative approaches of the other. The greatest risk is in creating an extreme scale system through error-prone PCR in Example 12. Specifically, it may be difficult to obtain high diversity of strands due to duplicates arising during the PCR process, and furthermore, strands obtained in the manner will be related through mutations so could physically interact with each other in unexpected ways that could have unknown effects on the system. To mitigate these risks, another Direct Evolution approach called ‘DNA shuffling’ (Werkman et al., (2011) Directed Evolution Through DNA Shuffling for the Improvement and Understanding of Genes and Promoters. In: Yuan L., Perry S. (eds) Plant Transcription Factors. Methods in Molecular Biology (Methods and Protocols), vol 754. Humana Press) that creates high diversity through swapping large segments of DNA between strands, rather than create individual mutations. Serial rounds of evolution are performed to increase the Hamming and Edit Distances between strands.

### Overview of Examples 13-14

Example 13 and 14 relate to approaches to computationally and experimentally estimate the maximum number of addresses possible. A long-standing challenge in molecular biology has been identifying design rules to predict whether a primer is ‘good’ and will bind specifically only to its perfect sequence match. In practice, biologists use some standard rules of thumb based upon experience and simple thermodynamic models to determine primer sequences, concentrations, and binding/annealing temperatures to use in PCRs. It is also common knowledge, that often PCR reactions do not work or give rise to non-specific products, and trial and error is also a large part of primer design. However, unlike biological research applications, a data storage system requires more stringent engineering. It is desired to accurately predict primers that work, and also predict what the maximum number of ‘good’ primers is as that places a hard limit on total system capacity. To address these challenges, computational and physical systems are developed that can directly measure key parameters of extreme scale DNA storage systems.

### Example 13

### Thermodynamically Driven Model and Simulation to Design DNA-DNA Interactions and Assess the Maximum Numbers of Primers Available

A collection of primers is designed to sample a large sequence space, as well as have subsets of primers be closely related to each other to study their interactions. Open source tools are leveraged to analyze primers individually and pairwise. For example, each primer can be analyzed independently for hairpins and homodimer formation (binding to itself). Also, each primer must be compared to every other primer to detect if non-specific binding is possible. In Examples 1-6, it was found that even primers with significant Hamming or Edit Distances (>9) are susceptible to nonspecific binding and must be analyzed. It is feasible to employ a greedy algorithm that builds a list of compatible primers with at least a constant (e.g. −10) ΔG difference between them all. A representative embodiment of a greedy algorithm to exhaustively search all length 20 primers currently attempts to select primers at a Hamming distance greater than 9, a ΔG of at least −10 to all other primers, and balanced GC content. The code is implemented in Python and runs as a set of distributed tasks on a high-performance computer (HPC) system, such as can be found at North Carolina State University, Raleigh, N.C., United States of America. Based on initial results, it is projected that the number of primers could be on the order of 10⁴ to 10⁶. See also FIG. 22A.

The current implementation is improved by accelerating key parts of the algorithm in C code, potentially allowing for an exhaustive search of the entire space in a relatively short period of time. The projected time is under two weeks on an HPC system. Repeated experiments are performed which randomly start with different sets of seed primers, to encourage the selection of different primer sets each run. From the accumulated results, the possible size of the maximum set of primers is statistically inferred. Finally, the threshold for ΔG is varied to determine how many primers can be selected under greater and less stringent thermodynamic constraints. Runs can also be varied to exclude Hamming and Edit Distance as factors in primer design and base decisions solely on their own thermodynamic properties.

### Example 14

### Massively Parallel “One-Pot” Approach to Measure a Large Sequence Space of DNA-DNA Interactions

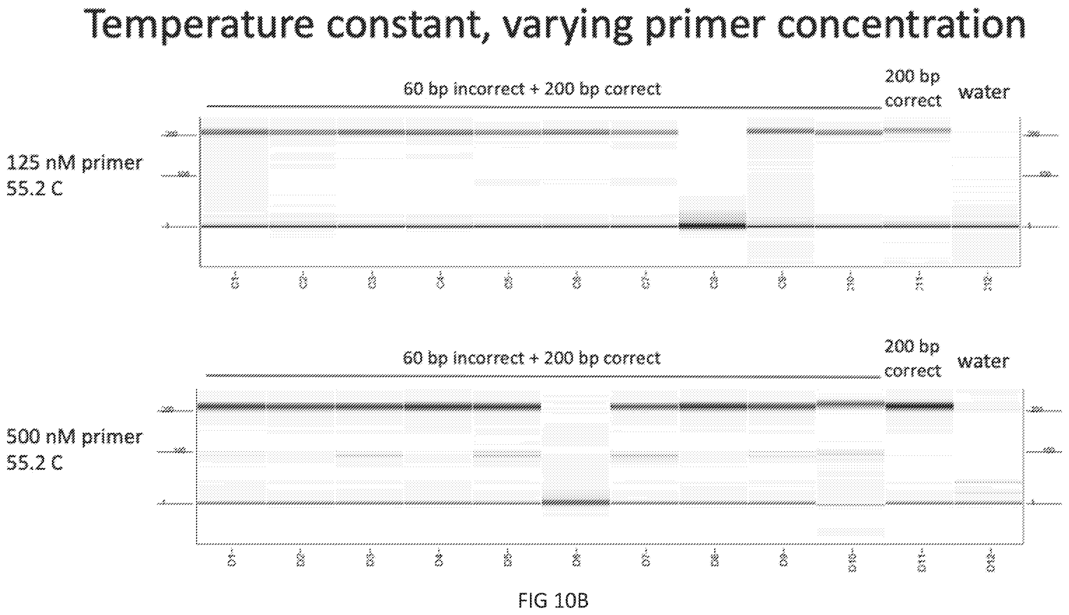

A library of 100 million primers as designed in Example 13 is ordered. Many of these primers have intentionally low ΔG and low Hamming Distance differences from each other. These are melted at 98° C. to ensure all primers are initially in a ssDNA form, then gradually the temperature is decreased to room temperature by 1° C. a minute. This allows primers to find as many binding partners as possible. Primers that bind together form dsDNA. Borrowing a technique from biochemistry and molecular biology, an enzyme called T7 DNA ligase is used to ligate the ends of the dsDNA primer pairs so they become one single piece of ssDNA that is hairpinned on itself. Next generation sequencing is performed, where the newly formed ssDNA will be read as one consecutive sequence. Therefore, primer sequences that show up on the same sequencing read (consecutive sequence/directly adjacent to each other) are indicative of primers that bound to each other. By analyzing the results, the computed result is compared with the experimental result and estimates for capacity are refined, as are thermodynamic binding models. See FIG. 9.

Alternative Approaches for Examples 13 and 14. Experimentally, the biggest risk is inefficient ligations, or over-representation of offtarget binding than what would occur in typical DNA storage systems. To address inefficient ligations, bound primers are isolated by gel electrophoresis to separate unbound ssDNA from bound dsDNA. It is also tested how well this system represents true off-target binding in practical storage systems by selecting a sample of 100 ‘good’ and 100 ‘bad’ primers and implementing them in a true 100,000 strand DNA storage system with random data payload sequences. The inherent risk of the models will be over- or underestimating the total number of primers possible. Experimental tests are iterated (between Examples 13 and 14) to refine the model and improve the estimate of total possible primers. An approach to further improve the model is to identify optimal hybrid contributions from ΔG and Edit/Hamming Distance.

### Overview of Examples 15-17

Examples 15-17 involve the performance of a search in storage through engineering binding interactions amongst DNA strands. With no electronic record as backup, searching a DNA library can be expensive and time consuming, requiring many files to be sequenced to find the one that is desired. By designing a short oligo that hybridizes to the sought-after data, that oligo can act as a query in solution, and the hybridized strands can be extracted as an answer to the query. Instead of reading all of the strands of one file, we sequence a few strands from lots of files, to find the desired files or data. Whereas Examples 7-9 leverage thermodynamics to make primers different from data to prevent unwanted binding and erroneous file access, now primers that are similar to data and data encodings are wanted. For such primers, ΔG and other thermodynamic properties are exploited to encourage binding not only with data that is an exact match data, but also data that is semantically similar to the desired answer; for example, a primer searching for a lower-case word will also match one that is uppercase because they have the same meaning. Computational tools are designed to construct such an encoding and experimentally verify that it works in a DNA-storage system in accordance with the presently disclosed subject matter. Furthermore, findings from Examples 7-9 are used control the degree of coupling it is desired for a search to have.

### Example 15

### Thermodynamically Driven Data Encodings for Efficient Search

Data encodings are optimized to enhance thermodynamic similarity between symbols that encode related information. Data is typically encoded such that each byte of raw data is a short fixed-length DNA sequence. It can then be chosen to encode bytes that have similar meaning with sequences that are thermodynamically similar. For example, capital ‘A’ and lower case ‘a’ could be given sequences that are very close thermodynamically. A primer for searching for the word “Abe” can be constructed that prefers capital ‘A’ but will also bind to lowercase ‘a’. However, a shortcoming of this approach is that search queries could at most encode 2 or 3 letters (length 20 primer divided by 6 bp/letter), limiting their usefulness. However, if the type of data encoded in a file is exploited to create denser encodings, for example word-based encodings, it may be possible to encode an entire dictionary in 10 nt and search for a two-word sequence, or use a longer primer and search with an even longer query. In this setting of word-based encodings, there are many relationships between words that can be exploited by assigning them thermodynamically coupled encodings. For example, synonyms can be grouped, terms often used together can be grouped, or related concepts can be grouped thermodynamically. The desired application-level and algorithm-level goals ultimately drive the choices made for grouping encodings.

A thermodynamically driven encoding algorithm is developed. Possible inputs to the algorithm include a set of symbols to be encoded, a set of relationships indicating which symbols should be thermodynamically similar and likely to bind, their strength of coupling, and a specification of the preferred thermodynamic gap between unrelated symbols. The output of the algorithm is a best-effort set of codewords that matches the desired constraints. The design of codewords should also account for known limitations in DNA sequencing and synthesis, like avoiding repeated bases in codewords, providing GCbalance, and coping with errors. To implement the algorithm, techniques are well known for global optimization problems, like simulated annealing or genetic algorithms to search the space of possible encodings and rank possible encodings according to the fitness metrics supplied as input. The algorithm is designed in a general way so as to facilitate re-use across a variety of search applications. See also FIG. 22B.

### Example 16

### Thermodynamically Encoded Data Along with Temperature and Concentration can Control the Degree and Quality of Search in Storage

A likely first algorithm is demonstrating performance of a synonym search over common English dictionary words. A book is encoded with each chapter in its own file. Search queries are designed that are known a priori to yield matches in the DNA library. A large set of queries, some which should match only one or a few strands, and others that should match many strands. Also, queries are designed that demonstrate either specific matches or non-specific matches based on variations in concentration and temperature of the primer. Using a similar methodology to Example 9, multiple PCRs are performed: ones at high temperature and low primer concentration should access only exact data matches; others at low temperature and high primer concentration should access all close matches within the encoded book. The results are sequenced and compared with computed predictions. The degree of specificity at the high temperatures and low temperatures is examined and compared with models to refine the algorithm developed in Example 15. Importantly, strands extracted from the search contain the index and primer sequences for the rest of the strands in the same file as the extracted strands. These can be used to obtain the remainder parts or content of any of the searched files.

### Example 17

### Performance and Efficiency Analysis of in Storage Search Compared with Random Access Driven Search

As a final step, a variety of conventional search algorithms are simulated operating on a DNA-based storage system and compared to techniques set forth in the Examples. For example, a linear search is performed through the entire storage system (inefficient), or an index of search terms is encoded as part of the storage that can used to quickly identify files of interest. The number of sequencing runs, the overhead of storage for the algorithm versus others including encoding overheads, the resilience to error, the cost, and overall execution time overhead are analyzed and compared. Because execution time will likely be dominated by sequencing time, analytical models may be sufficient for performance comparison. If needed, detailed computer system simulations using architectural and storage system simulators are implemented and analyzed.

### Example 18

### Process and System for Dynamic DNA-based Information Storage

This Example describes a process and system in accordance with the presently disclosed subject matter. In some aspects, temperature optimizations allowed for the performance of many of the steps at or near room temperature and for the avoidance of repeated cycling of temperatures such as required in PCR-based file-access methods common to other DNA-storage systems. Isothermal and room temperature operation helps to reduce the complexity of a DNA-based information storage device, and also increases the stability and longevity of the DNA because it does not add to high temperatures as often. The process and system is referred to collectively as DORIS (Dynamic Operations and Reusable Information Storage) for convenience.

### Methods:

Creation of Toehold Strands: Toehold strands were created by “filling in” ssDNA templates (IDT) with primer TCTGCTCTGCACTCGTAATAC (SEQ ID NO: 1, Eton Biosicence, San Diego, Calif., United States of America) at a ratio of 1:40 using 0.5 μL of Q5 High-Fidelity DNA Polymerase (NEB, Ipswich, Mass., United States of America; M0491S) in a 50 μL reaction containing 1×Q5 polymerase reaction buffer (NEB, Ipswich, Mass., United States of America; B9072S) and 2.5 mM each of dATP, dCTP, dGTP, and dTTP (all from NEB, Ipswich, Mass., United States of America; N0440S, N0441S, N0442S, and N0443S, respectively). The reaction conditions were 98° C. for 30 seconds and then 4 cycles of: 98° C. for 10 seconds, 53° C. for 20 seconds, 72° C. for 10 seconds, with a final 72° C. extraction step for 2 minutes. Toehold strands were purified using AMPure XP beads (Beckman Coulter, Brea, Calif., United States of America; A63881) and eluted in 214 of water.

File Separations: Oligos were purchased with a 5′ biotin modification (Eton Bioscience, San Diego, Calif., United States of America). Toehold strands were diluted to 10¹¹ strands and mixed with biotinylated oligos at a ratio of 1:40 in a 50 μL reaction containing 2 mM MgCl₂ (Invitrogen, Carlsbad, Calif., United States of America; Y02016) and 50 mM KCl (NEB, Ipswich, Mass., United States of America; M0491S). Oligo annealing conditions were 45° C. for 2 minutes, followed by a temperature drop at 1° C./minute to 14° C. Streptavidin magnetic beads (NEB, Ipswich, Mass., United States of America; S1420S) were prewashed using high salt buffer containing 20 mM Tris-HCl, 2 M NaCl, and 2 mM EDTA at pH 8 and incubated with toehold strands at room temperature for 30 minutes. The retained library was recovered by collecting the supernatant of the separation. The beads were washed with 100 μL of high salt buffer and used directly in the in vitro transcription reaction. After transcription, the beads with the bound files were washed twice with 100 μL of low salt buffer containing 20 mM Tris-HCl, 0.15 M NaCl and 2 mM EDTA at pH 8 and subsequently eluted with 95% formamide (Sigma, St. Louis, Mo., United States of America; F9037) in water. The quality and quantity of the DNA in the retained library and file were measured by quantitative real time PCR (Bio-Rad Laboratories, Hercules, Calif., United States of America).

In vitro Transcription: Immobilized toehold strands bound on the magnetic beads were mixed with 30 μL of in vitro transcription buffer (NEB, Ipswich, Mass., United States of America; E2050) containing 2 μL of T7 RNA Polymerase Mix and ATP, TTP, CTP, GTP, each at 6.6 mM. The mixture was incubated at 37° C. for 8, 16, 32, and 48 hours, followed by a reannealing process where the temperature was reduced to 14° C. at 1° C./minute to enhance the retention of toeholds on the beads, the newly generated RNA transcripts were separated from the streptavidin magnetic beads and their quantity measured using the Qubit RNA HS Assay Kit (ThermoFisher Scientific, Waltham, Mass., United States of America; Q32852).

Reverse Transcription: First-strand synthesis was generated by mixing 5 μL of separated RNA transcript with 500 nM of reverse primer in a 20 μL reverse transcription reaction (Bio-Rad Laboratories, Hercules, Calif., United States of America; 1708897) containing 4 μL of reaction supermix, 2 μL of GSP enhancer solution and 1 μL of reverse transcriptase. The mixture was incubated at 42° C. for 30 or 60 minutes, followed by a deactivation of the reverse transcriptase at 85° C. for 5 minutes. The resultant cDNA was diluted 100-fold, and 1 μL was used as the template in a PCR amplification containing 0.5 μL of Q5 High-Fidelity DNA Polymerase (NEB, Ipswich, Mass., United States of America; M0491S), 1×Q5 polymerase reaction buffer (NEB, Ipswich, Mass., United States of America; B9072S), 0.5 μM of forward and reverse primer, 2.5 mM each of dATP, dCTP, dGTP, and dTTP (each from NEB, Ipswich, Mass., United States of America; N0440S, N0441S, N0442S, and N0443S, respectively) in a 50 μL total reaction volume. The amplification conditions were 98° C. for 30 seconds and then 25 cycles of: 98° C. for 10 s, 55° C. for 20 seconds, 72° C. for 10 seconds with a final 72° C. extension step for 2 minutes. The quality of amplification was measured using gel electrophoresis.

Locking and Unlocking: Lock and key strands were purchase from Eton Bioscience (San Diego, Calif., United States of America). To lock the file, purified toehold strands were mixed with lock strands at a molar ratio of 1:10 in a 25 μL reaction containing 2 mM MgCl₂ and 50 mM KCl. The mixture was annealed to 98° C., 45° C. or 25° C. for 2 minutes, followed by a temperature drop at 1° C./minute to 14° C. To unlock the file, key strands were added into the locked file mixture at a molar ratio of 10:1 to the original toehold strand amount. The mixtures were annealed to 98, 77, 55, 35, or 25° C. for 2 minutes, followed by a temperature drop at 1° C./minute to 14° C. To access the unlocked strands, file specific biotin-modified oligos were added into the mixture at a ratio of 15:1 to the original toehold strand amount supplemented with additional MgCl2 and KCl to a final concentration of 2 mM and 50 mM, respectively, in a 30 μL reaction.

Renaming and Deleting: Toehold strands were mixed with renaming or deleting oligos at a ratio of 1:20 in a 25 μL reaction containing 2 mM MgCl₂ and 50 mM KCl. The mixture was heated to 35° C. for 2 minutes, followed by a temperature drop at 1° C./minutes to 14° C. To delete the file, oligos were mixed with purified target file strands at a ratio of 1:20.

Real-Time PCR (qPCR): qPCT was performed in a 6 μL, 384 well plate format using SsoAdvanced Universal SYBR Green Supermix (BioRad Laboratories, Hercules, Calif., United States of America; 1725270). The amplification conditions were 95° C. for 2 minutes and then 50 cycles of: 95° C. for 15 seconds, 51.5° C. for 20 seconds, and 60° C. for 20 seconds. Quantities were interpolated from the linear ranges of standard curves performed on the same qPCR plate.

Theoretical Thermodynamic Calculations: To theoretically estimate the fraction of bound oligos with various overhang lengths and at different temperatures, the equilibrium constants were calculated at each condition:

K=exp(−ΔG/RT)

where ΔG⁰ is the change in Gibbs Free Energy at standard conditions (25° C., pH=7 in this case; R is the gas constant, and T is the reaction temperature. The Gibbs Free Energy for each oligo was obtained using the Oligonucleotide Properties Calculator. See Sugimoto et al., Nucleic Acids Res. 24, 4501-4505 (1996); Kibbe, Nucleic Acids Res. 35, W43-W46 (2007); and Lomzov et al., J. Phys. Chem. B 119, 15221-15234 (2015). The equilibrium constant at each condition was equated to:

K=[Oligo−Toehold Strand]/([Toehold Strand]×[Oligo])

with

[Oligo−Toehold Strand]/[Toehold Strand]=K×[Oligo]

representing the fraction of accessed strands (strands separated out) to the total original amount of toehold strands. This amount, expressed as a percentage, is referred to as the access efficiency.

Density and Capacity Calculation: Experimental work was performed using the oligos listed in Table 5 below. Simulation densities were measured by calculating the number of bytes in a 160 bp data payload with 5 codewords used for the strand index (see Organick et al., Nat. Biotechnol. 36, 242-248 (2018)), with the codeword length given as L:

Density=(160−5 ×L)/L

The size of the index is chosen to accommodate 10⁹ strands.

TABLE 5
Template and oligo design

| DNA Oligo | Sequence |
|---|---|
| ssDNA (File A) | CGTACGTACGTACGTCGACGGATGACAGCTCGCATCTACGAGCT |
| | CGAGATGACACAGAGTATCGCATCTACGACACAGTCTCTCGCGA |
| | GCTAGAGATGAGTGATCGAGCTCTGCTCGGCGCGCTATAGTGAG |
| | TCGTATTACGAGTGCAGAGCAGACTCAC (SEQ ID NO: 2) |
| ssDNA (File A-2 | CGTACGTACGTACGTCGACGGATGACAGCTCGCATCTACGAGCT |
| for Truncated | CGAGATGACACAGAGTATCGCATCGAGTGCAGAGCAGACTCACA |
| PCR) | GCTAGAGATGAGTGATCGAGCTCTGCTCGGCGCGCTATAGTGAG |
| | TCGTATTACGAGTGCAGAGCAGACTCAC (SEQ ID NO: 3) |
| ssDNA (File B) | CAGGTACGCAGTTAGCACTCCGTACGTACGTACGCAGCTAGCTC |
| | GATGAGTACTCTGCTCGATGAGTACTCTGCTCGACGAGATGAGA |
| | CGAGTCTCTCGTAGACGAGAGCAGACTCAGTCATCGCGCTAGAG |
| | AGCATAGAGTCGTGATCTATGCTCAGCGCGCTATAGTGAGTCGT |
| | ATTATCCGTAGTCATATTGCCACG (SEQ ID NO: 4) |
| ssDNA (File C) | GGGAGTAATCCCCTTGGCGGTCGCGGGGGACAGCGCGTACGTGC |
| | GTTTAAGCGGTGCTAGAGCTGTCTACGACCAGCGCGCGCTATAG |
| | TGAGTCGTATTAGGATTCTCCAGGGCATCCGG (SEQ ID NO: |
| | 5) |
| Extension Primer | TAATACGACTCACTATAGCGCGC (SEQ ID NO: 6) |
| File Access Oligo | GTGAGTCTGCTCTGCACTCG (SEQ ID NO: 7) |
| A′ | |
| File Access Oligo | CGTGGCAATATGACTACGGA (SEQ ID NO: 8) |
| B′ | |
| File Access Oligo | CCGGATGCCCTGGAGAATCC (SEQ ID NO: 9) |
| C′ | |
| PCR Oligo B | CTACGACACAGTCTCTCGCG (SEQ ID NO: 10) |
| Reverse Oligo | CGTACGTACGTACGTCGACG (SEQ ID NO: 11) |
| File A | |
| Reverse Oligo | CAGGTACGCAGTTAGCACTC (SEQ ID NO: 12) |
| File B | |
| Reverse Oligo | GGGAGTAATCCCCTTGGCGGT (SEQ ID NO: 13) |
| File C | |
| cDNA Forward | CGTACGTACGTACGTCGACG (SEQ ID NO: 14) |
| Oligo | |
| cDNA Reverse | GAGCAGAGCTCGATCACTCA (SEQ ID NO: 15) |
| Oligo | |
| File A Lock | CTCCATCAGAGTGATATGCCCAGCTTAGGTGAGTCTGCTCTGCA |
| | CTCG (SEQ ID NO: 16) |
| File A Key | CGAGTGCAGAGCAGACTCACCTAAGCTGGGCATATCACTCTGAT |
| | GGAG (SEQ ID NO: 17) |
| File A -> B | TCCGTAGTCATATTGCCACGGTGAGTCTGCTCTGCACTCG |
| Rename Oligo | (SEQ ID NO: 18) |
| File A -> C | GGATTCTCCAGGGCATCCGGGTGAGTCTGCTCTGCACTCG |
| Rename Oligo | (SEQ ID NO: 19) |
| File A Delete | GTGAGTCTGCTCTGCACTCG (SEQ ID NO: 20) |
| Oligo | |

Capacity: For each density and corresponding number of oligos, system capacity is calculated assuming 10⁹ strands per file, which roughly corresponds to the number of strands that can be sequenced at a time in next generation sequencing. It was assumed that each strand occurs 10 times in the replicate,

Capacity=10⁹×(number of primers)×Density/10

The capacity calculations are based on the number of oligos found in the search, not the total number that could be available by searching the entire space of all possible 20 bp oligos. Note, these capacity calculations are based on the number of oligos found in a search (FIGS. 22A and 22B), not the total number that may be available if the entire space of all possible 20 bp oligos was searched. Searching for more oligos will result in greater system capacity.

Referring to FIG. 22A, the overall process was a loop that repeated over some number of attempts to find a primer. Primer sequences were generated at random according to a uniform distribution of A, C, G, and T. Then, each primer was evaluated against several criteria. For estimating Tm (melting temperature), hairpins, and other dimer formation, the Primer3 software was used (Untergasser A, et al., Nucleic Acids Res. 2012; 40(15):e115-e115). It was required that all primers were at least a Hamming distance of 6 apart, and it was required that they were at least a Hamming distance of 6 away from the target library. It was approximated that requirement by comparing each primer to 1 MB of data that was randomly generated and encoded in each run of the program. The encoding of the library was also an input to the process that could be varied to evaluate the impact of coding density on primer selection.

Referring now to FIG. 22B, the data payload of strands, used to validate primers in the flowchart on the left, were created by encoding each byte one at a time as a codeword. The codeword tables at various lengths were created through a common algorithm. For length 4, all possible sequences are used, hence the creation is trivial. For length 5, all possible sequences are generated, and 256 of them are selected at random. For lengths of 6 or longer, the process is different. Codes are generated in base-3 (ternary) and are selected, one for each possible single byte value. To ensure the codewords were different enough from each other, it is first attempted to generate codes of maximal Hamming distance according to the Singleton bound. If this does not succeed, the distance is reduced and the process is tried again. The codeword tables created by the algorithm were manually verified to have a distance of 2 or more for all lengths greater than 6. For length 6 and higher, after encoding an entire strand, a rotating encoding is used to ensure no repetitions in the strand, neither within nor across codewords.

### Discussion:

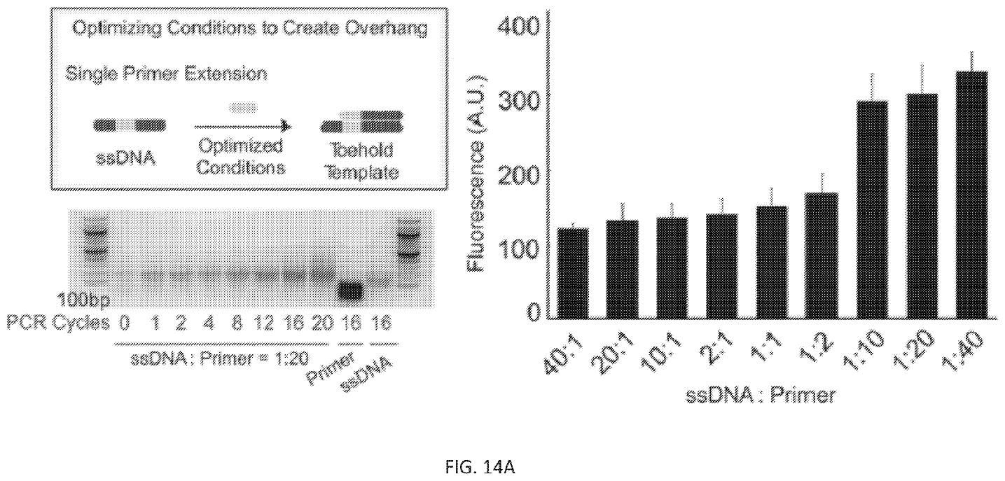

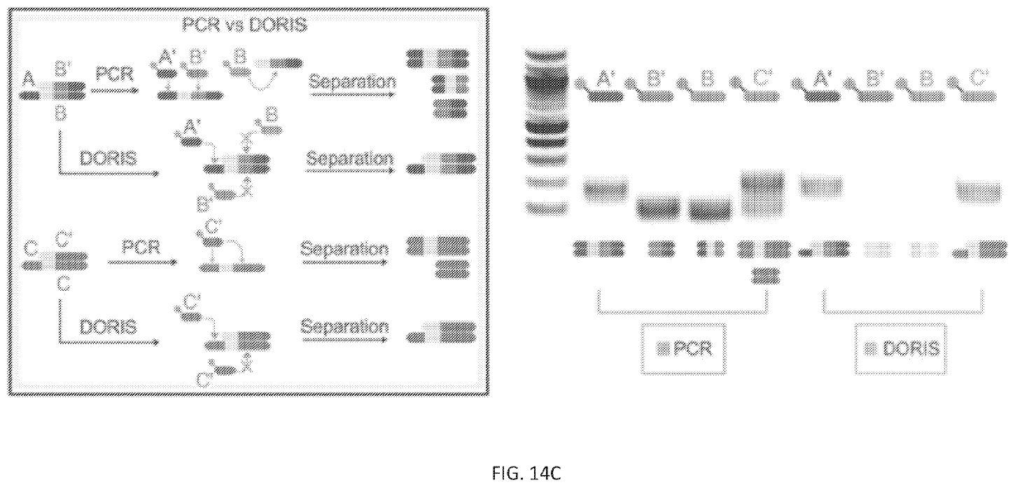

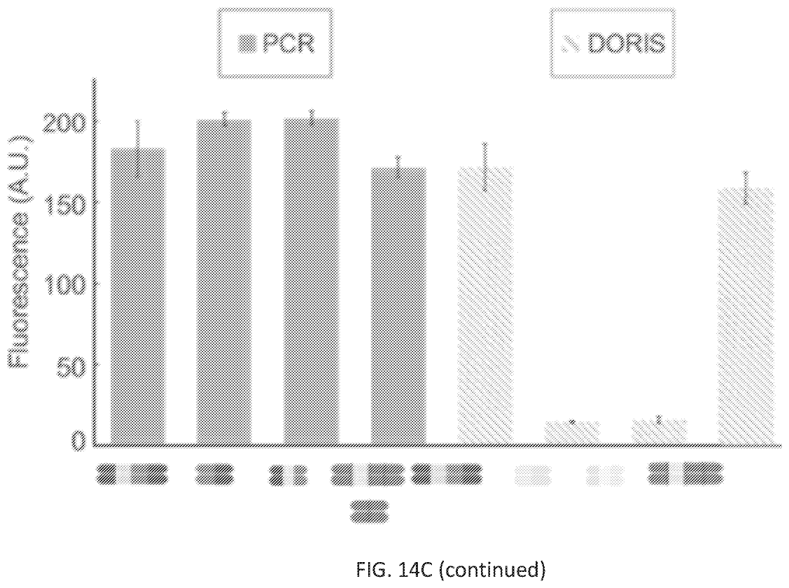

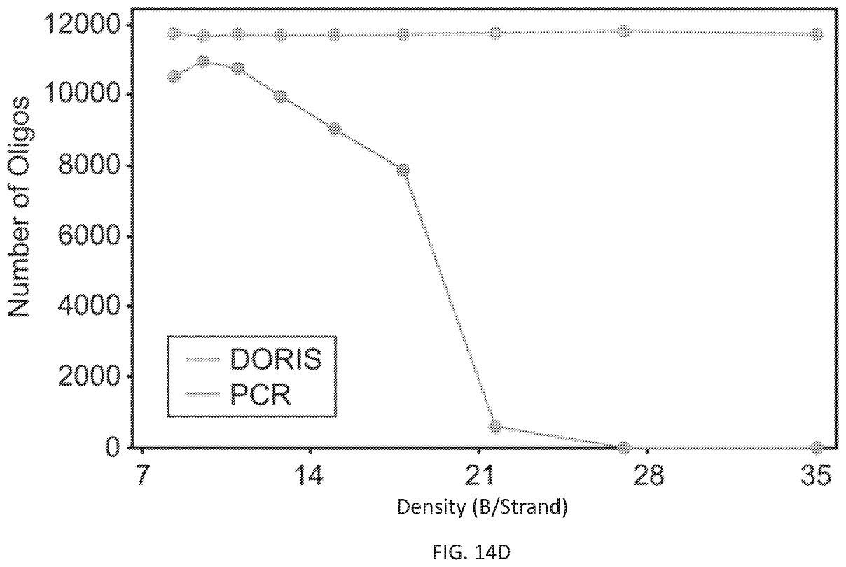

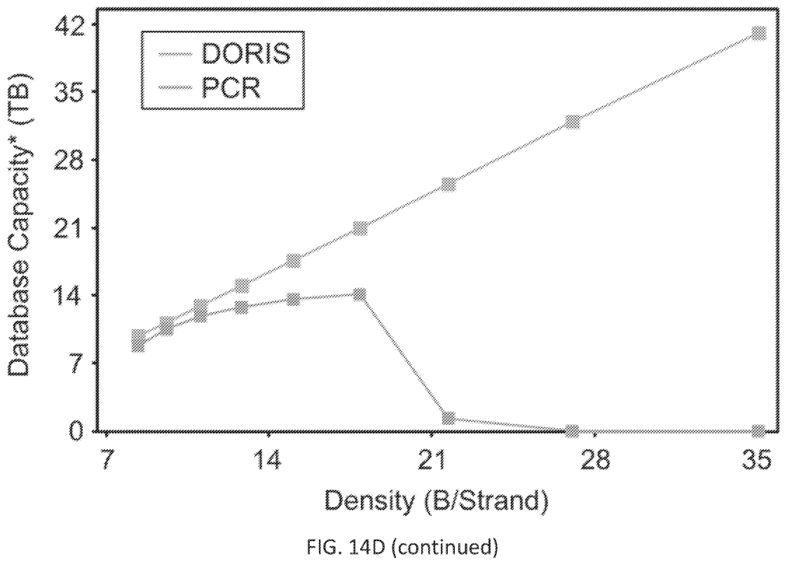

FIG. 14A shows the creation of an overhang/toehold structure for DNA strands. The toeholds serve as an architectural feature of the presently disclosed systems where DNA strands are comprised of dsDNA with 3′ ssDNA overhangs. See also FIG. 12C. Toeholds can serve as multi-purpose structures serving as file addresses as well as molecule “handles” for file operations. 160 bp ss DNA strands with a common 23 bp sequence inset 20 bp from the 3′ end were used. See FIG. 14A, top left. This 23 bp sequence contains the T7 RNA polymerase binding sequence (allowing data access directly by T7-based in vitro transcription (IVT)). In the system shown in FIG. 14A, the sequence was used to bind a primer that is common to all strands in a database, followed by several cycles of thermal annealing and DNA polymerase extension (e.g., “PCR cycles”, but with only one primer), resulting in toehold strands with a 20 bp 3′ss DNA overhang. The ratio of ssDNA to primer, the number of cycles, and other parameters were optimized to maximize the amount of ssDNA converted to toehold structures. In particular, by decreasing the ssDNA:primer ratio past 1:10 led to a step change in the amount of toeholds produced as quantified by gel electrophoresis. See FIG. 14A, bottom left and right. Accordingly, a 1:20 ssDNA:primer ratio was used in further studies, as at that ratio only 4 PCR cycles were needed to convert the ssDNA into toehold structures, as seen by the upward shift in the DNA gel. See FIG. 14A, bottom left.

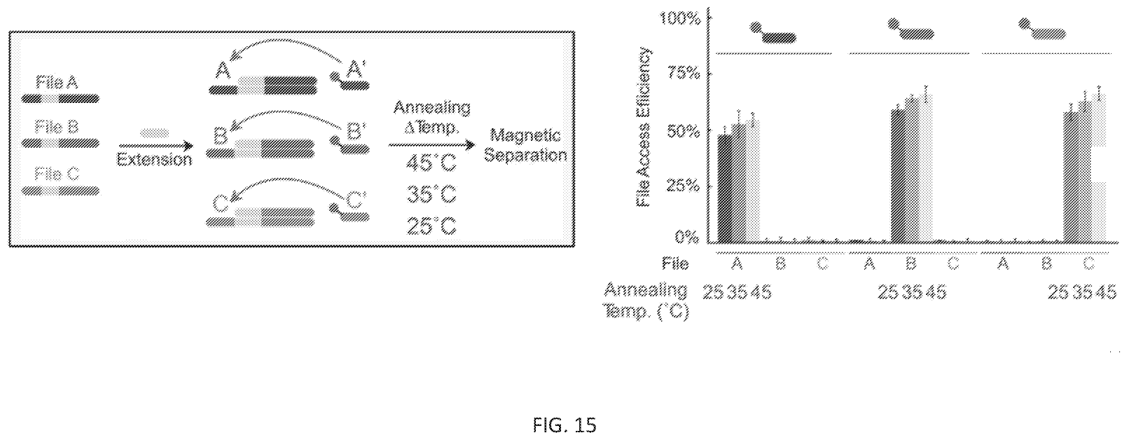

This method was then used to create 3 distinct toehold strands in one pot and the mixture was tested to see if each strand could then be specifically separated from the mixture. See FIG. 14B. More particularly, 3 distinct ssDNAs, “A”, “B”, and “C” were mixed together, T7 primer was added and 4 PCR cycles were performed. Biotin-linked, 20 bp DNA oligos were used to bind each strand (i.e., “file”) and separate them out from the mixture using streptavidin-linked magnetic beads. Each of these “file-access” oligos were able to specifically pull out only their corresponding strands without the other two. See FIG. 14B, bottom. The file separation step did not require repetitive and high temperature annealing steps, and was performed at room temperature (25° C.) with only minimal gains observed at higher oligo annealing temperatures of 35 or 45° C. See FIG. 15.

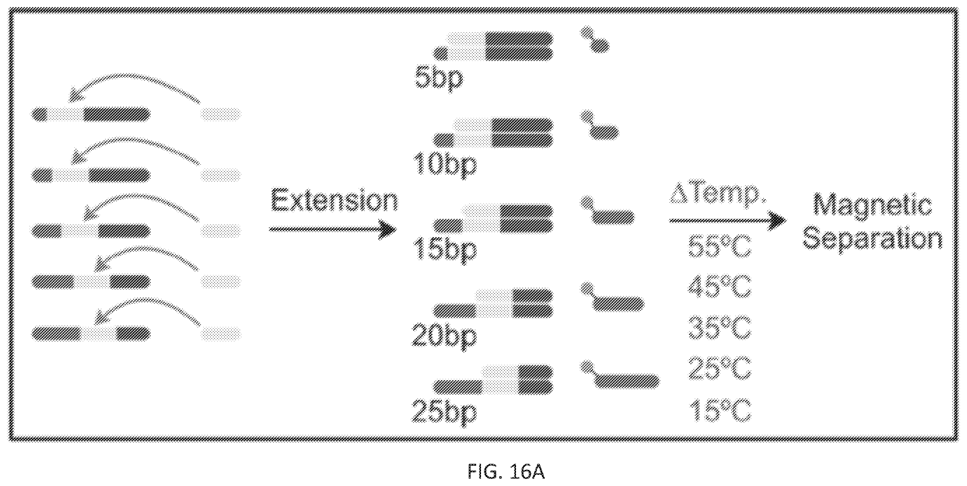

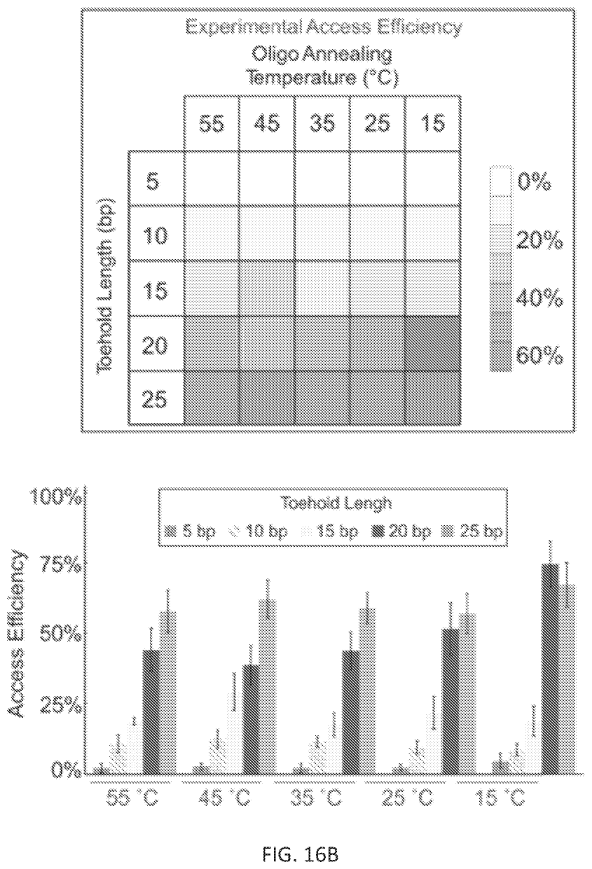

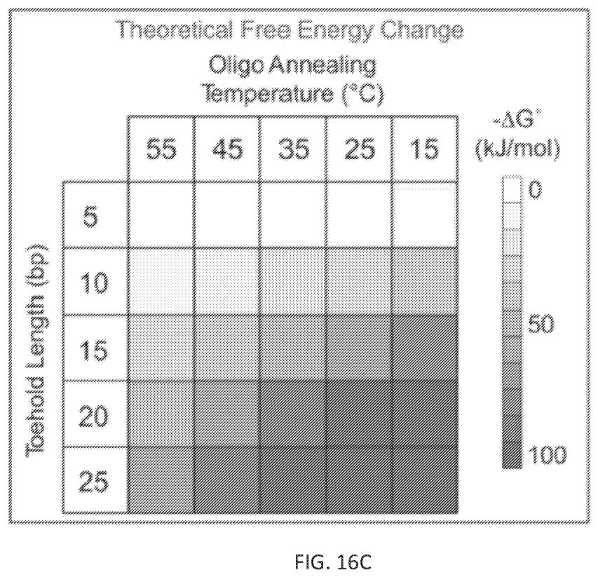

While 20 bp is a standard DNA primer or oligo length, the effects of toehold length and access temperate on file access efficiency were also studied. Five strands with 5-25 bp toeholds were designed. See FIG. 16A. Each strand was accessed using its specific biotin-linked oligo and magnetic separation was performed at 15-55° C. Enhanced access efficiency was observed for longer toehold sequences (20 and 25 bp) and at lower temperatures. See FIG. 16B. This was in agreement with a thermodynamic analysis. See FIG. 16C.

Without being bound to any one theory, it is believed that direct access of files through toehold structures can provide an advantage over PCR-based file access. For example, by eliminating the need to thermally anneal the system, the strands do not denature and can act as a natural barrier to oligos binding non-specifically within data payload regions. This could increase the theoretical information density and capacity of storage systems by allowing sequences similar to the oligos (file addresses) to appear in data payloads. To compare DORIS with PCR-based access, two toehold strands were created. See FIG. 14C, left. One strand hand an internal binding site for oligo B′ and a toehold that bound oligo A′. Using DORIS, only oligo A′ but not oligo B′ can access the strand. See FIG. 14C, middle and right. In contrast, when PCR was used, both oligo A′ and oligo B′ accessed the strand with oligo B′ producing undesired truncated products. The second strand that was tested had an internal binding site and toehold that both bound oligo C′. Using DORIS with oligo C′ separated out the full-length strand. In contrast, when using PCR, oligo C′ created both full length and truncated sequences.

To further assess the impact of the increased file specificity that DORIS provides, Monte Carlo simulations were performed to estimate the total number of oligo sequences and total capacities achievable when oligo sequences were or were not prohibited from appearing in the data payload regions. See FIG. 14D. The data payload region can be made more or less diverse in sequence, which corresponds to more or less information density, respectively. However, with increased diversity and density, the likelihood of an oligo sequence appearing in the data payload increases. For PCR (but not DORIS), this reduces the number of primers available to be used, leading to a reduction in total system capacity. It was found that capacity monotonically increases with increasing density for DORIS. In contrast, for PCR, increasing density initially provides a minor benefit to overall capacity, but eventually leads to a catastrophic drop in capacity as the number of non-conflicting primers quickly drops to zero. It is possible to increase the number of strands per file as encoding density is increased to make up for the loss of primers. However, this runs counter to the goals of random access since it can result in files too large to sequence and decode in a single sequencing run. These simulations were based upon conservative database sizes of only 10⁹ DNA strands, while future storage systems are likely to exceed 10¹² strands or greater. As database sizes and the total amount of DNA sequence space increase, the number of primers available for PCR-based systems will drop even further while DORIS will remain unaffected. Therefore, the theoretical capacity and density improvements DORIS provides could be orders of magnitude greater than what is estimated in the present simulation.

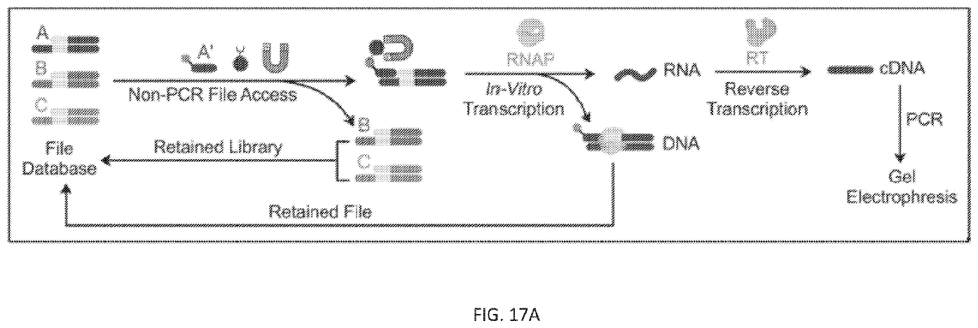

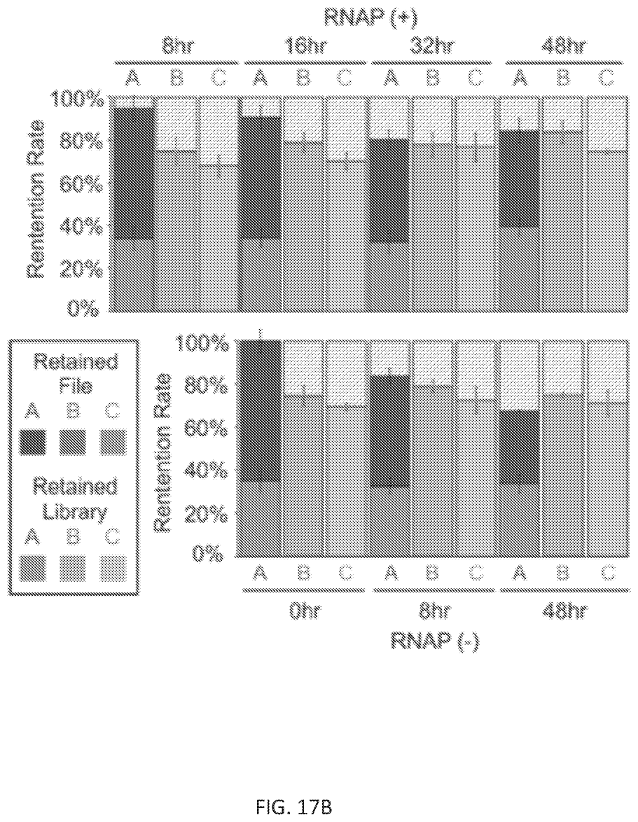

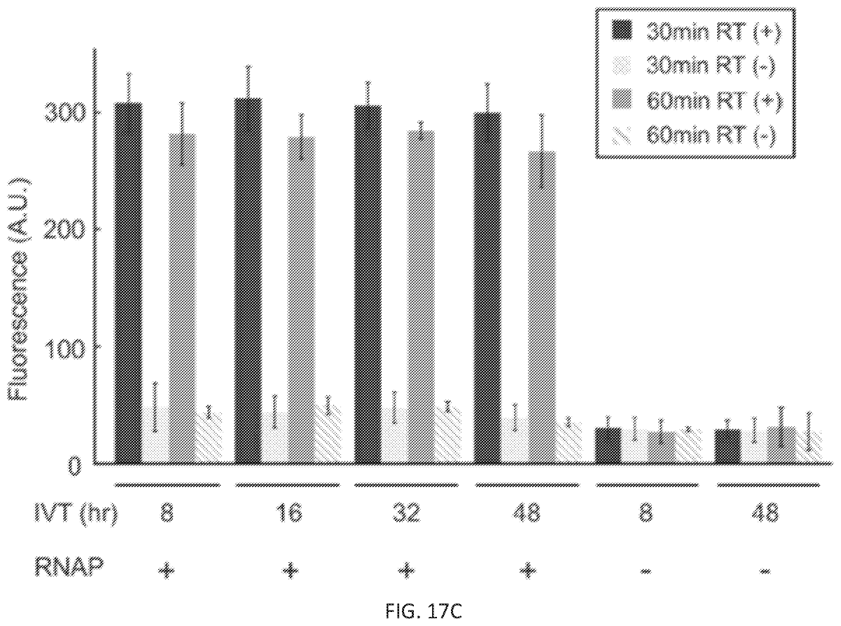

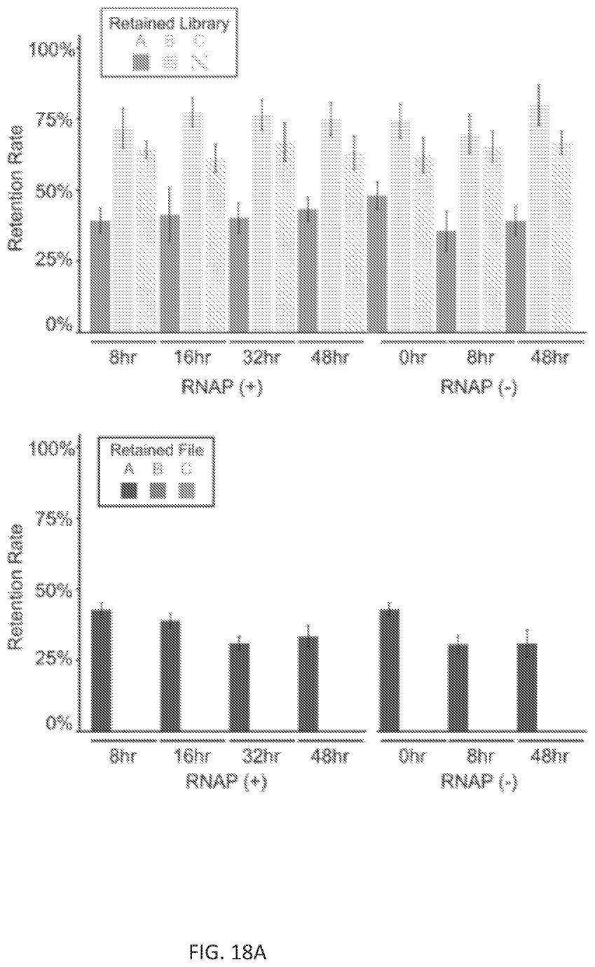

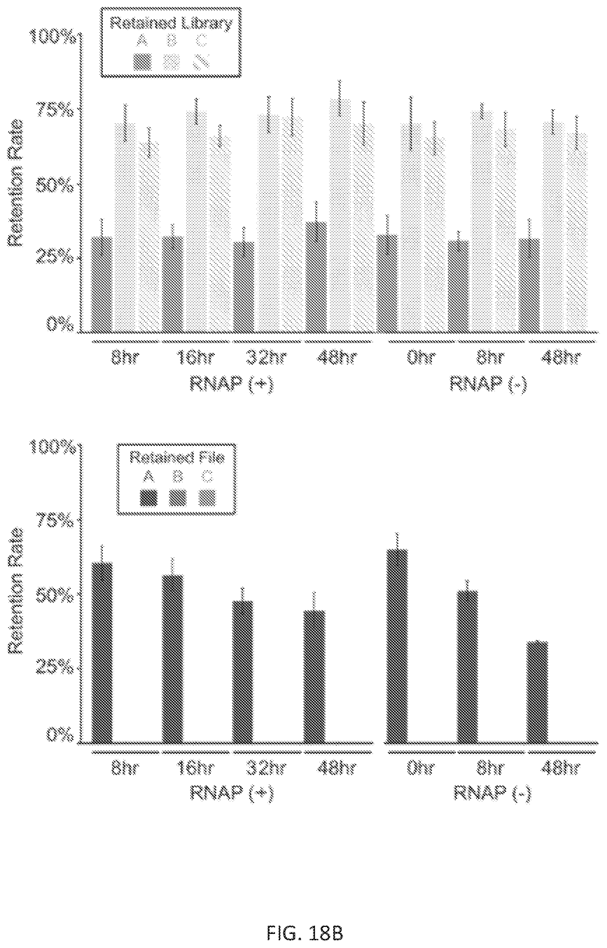

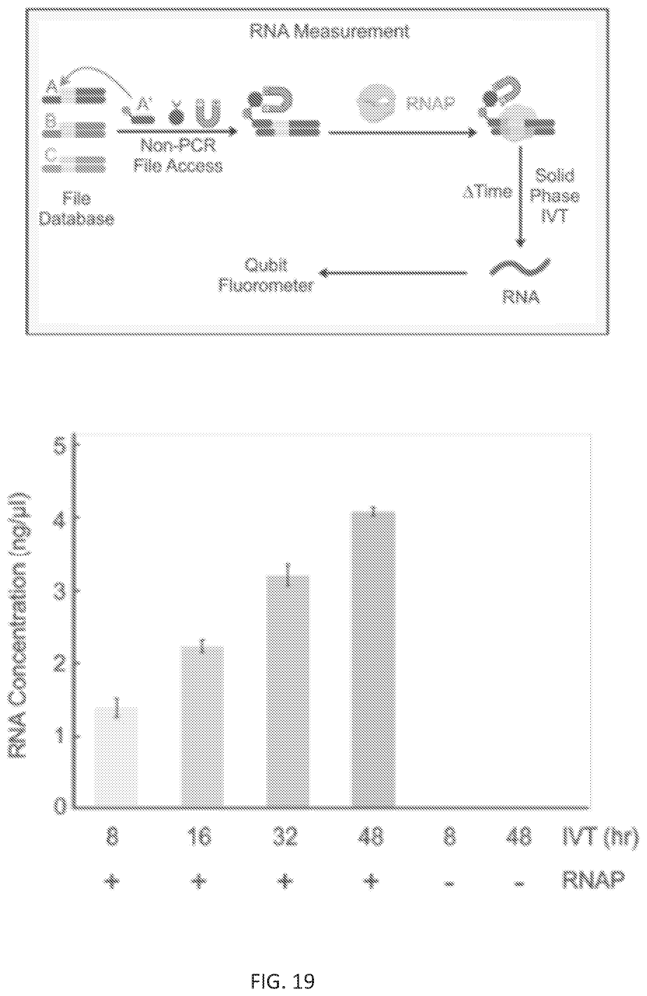

Since it would be preferable to return a file to the database for future use, studies were conducted to determine how to access a file's information without needing to destroy the file itself through the DNA sequencing process. In natural biological systems, information is repeatedly accessed from a single permanent copy of genomic DNA through the transcription process. Accordingly, a T7 promoter was designed to be common to all strands and to serve to synthesize the toe hold structures and also be able to initiate transcription. FIG. 17A shows a system wherein a file of interest is physically retrieved using biotin-linked oligos and streptavidin coated magnetic beads, RNA is in vitro transcribed directly from the bead-coupled file, the file is returned to the library, and the RNA is reverse transcribed into cDNA for downstream analysis or sequencing. The system was implemented with 3 distinct toehold strands representing a file database and file A was accessed with a biotin-linked oligo A′. The amounts and compositions of the “retained library” and the “retained file” were determined. See FIG. 17B. The retained library had higher levels of files B and C compared to A, as some of the file A strands had been magnetically removed. The retained file contained only file A strands, with no B or C. The net total amount of file A recovered from the retained library and retained file was 95% of what was originally in the database, suggesting that the loss of file A throughout the process was minimal. The length of time of the IVT step was studied to see if it affected the amount of recovered DNA, as IVT operates at an elevated temperature of 37° C. and could destabilize DNA. There was a slight trend of DNA loss with longer incubation times of up to 48 hours. See FIG. 18A. However, this loss can be improved by adding an annealing step after IVT. See FIG. 18B. The retrieval of information from file A following IVT by reverse transcription and PCR amplification was studied. Increasing IVT times from 8 to 48 house improved the yield of RNA (see FIG. 19), but the reverse transcription step appeared to be saturated as it did not improve the yield of dsDNA subsequently created. See FIG. 17C. Therefore, 8-hour IVTs are believed sufficient for maximum file access. In addition, 60-minute reverse transcriptions had no beneficial effects over 30 minutes. Finally, leaving out either T7 RNA polymerase or reverse transcriptase abrogated file access, indicating that the information recovered was specifically derived from RNA production and not due to any contaminating strands from the original database. See FIG. 17C. Thus, overall, DORIS is able to access specific information while retaining significant amounts of the original database and accessed.

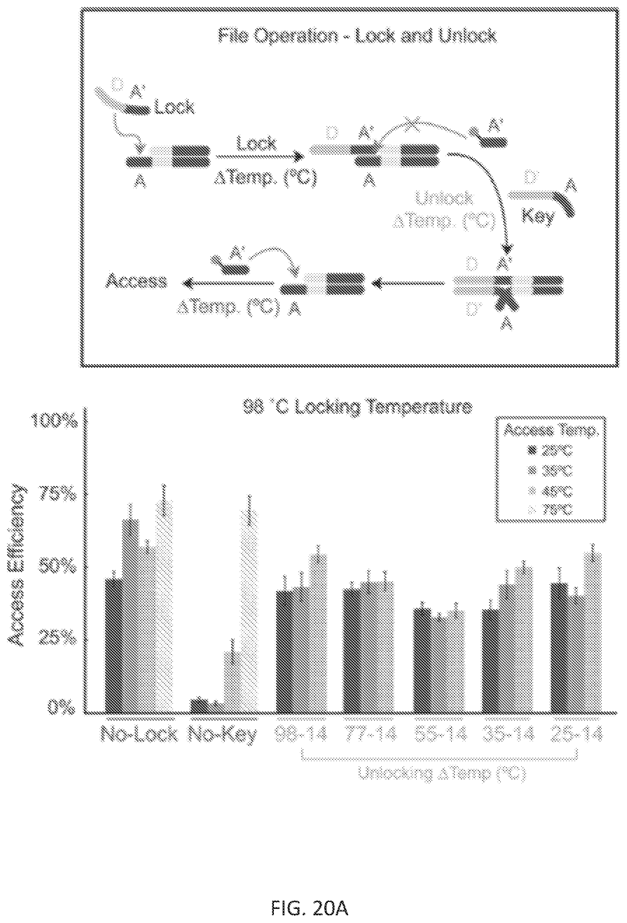

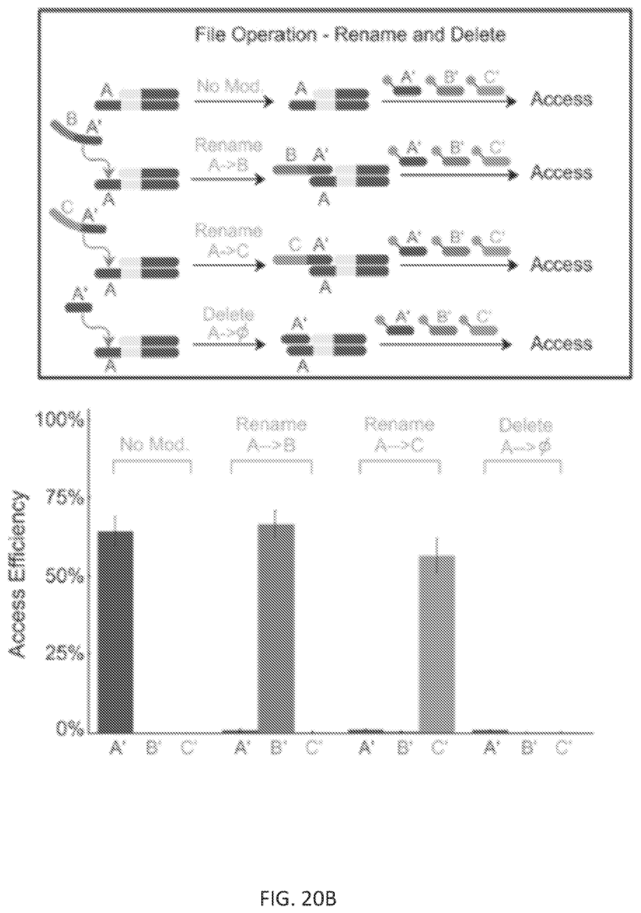

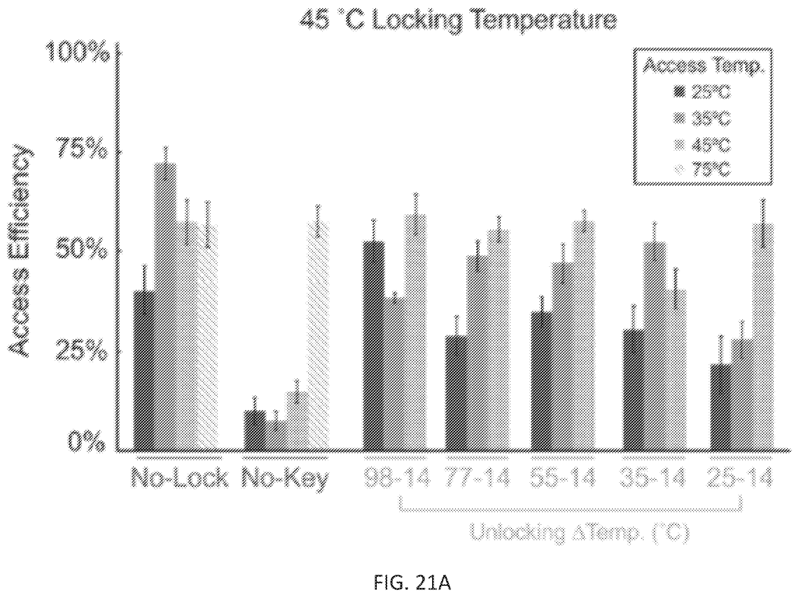

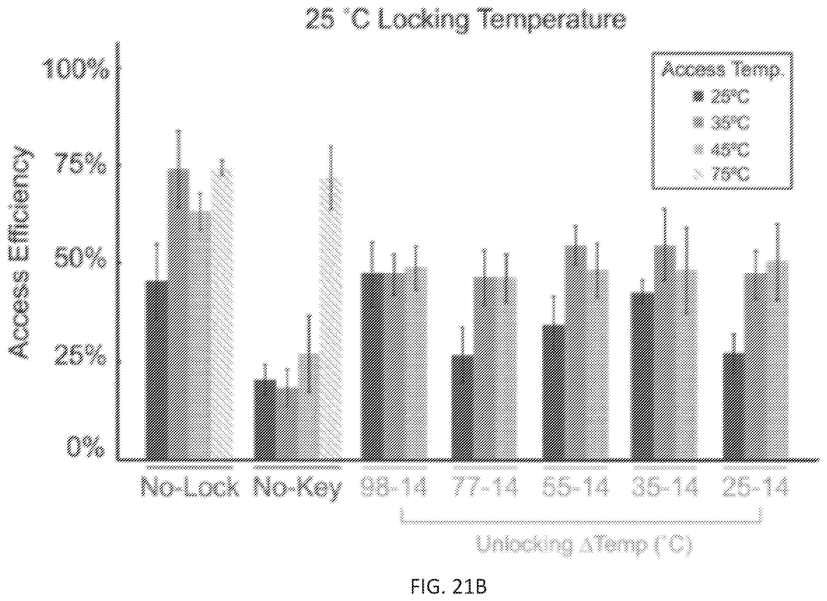

Many organic information storage systems, even cold storage archives, maintain the ability to dynamically manipulate files. Similar capabilities in DNA-based systems can significantly enhance their value and competitiveness. As a proof-of-principle, locking, unlocking, renaming, and deleting file operations were implemented and it was determined that these operations could be performed at room temperature. See FIGS. 20A, 20B, 21A, and 21B.

More particularly, a 3-file database was tested for the ability of a biotin-linked oligo A′ to bind and access file A at a range of temperatures from 25 to 75° C. See FIG. 20A, bottom, “no-lock”). File A was successfully accessed at all temperatures with roughly 50% of its strands accessed. To lock file A, file A was extracted from the 3-file database and mixed in a long 50 bp ssDNA “lock” that had a 20 bp complementary sequence to the toehold of file A. With the lock in place, oligo A′ was no longer able to access the file except at higher temperatures above 45° C. (“no-key”). Without being bound to any one theory, this is believed to be because the lock was melted from the toehold, allowing for oligo A′ to compete for toehold binding. To unlock the file, a “key” that was a 50 bp ssDNA fully complementary to the lock was added. See FIG. 20A, top. Different unlocking temperatures were tested, and it was found that the key was able to remove the lock at room temperature with the same efficiency as at high temperatures. Without being bound to any one theory, this is likely due to the long 30 bp toehold presented by the lock, allowing the key to “unzip” the lock from file A. In addition, the relative molar ratios (file A:lock:key:oligo A′=1:10:10:15) were optimized to minimize off-target access and ensure proper locking. The temperature at which the lock was added appeared to influence the fidelity of the locking process. At 98° C., the locking process worked well. When the lock was added at 25° C., there was leaky access even when no key was added. See FIG. 21B. Without being bound to any one theory, this could be due to secondary structures preventing some file A strands from hybridizing with locks at low temperatures. Fortunately, locking at 45° C. had reasonable performance (see FIG. 21A), thus avoiding the need to elevate the system to 95° C. In the context of a further embodiment of a DNA storage system, files could first be extracted then locked or renamed at an elevated temperature, then returned to the database, thus avoiding exposure of the entire database to elevated temperatures. The entire process could otherwise be performed at room temperature.

File renaming and deletion operations were also implemented. To rename a file with address A to have address B, file A was mixed with 40 bp ssDNA that binds to A, with the resultant toehold being address B. See FIG. 20B, top. All components were added at similar ratios to the locking process (file:renaming oligo:accessing oligo=1:10:15) and the renaming oligo was added at 45° C. The number of file strands each oligo A′, B′, and C′ could access was tested, and it was found that the renaming process completely blocked oligos A′ or C′ from accessing the file. See FIG. 20B, bottom. Only oligo B′ was able to access the file, indicating that almost all strands were successfully renamed from A to B. Similarly, file A could be successfully renamed to file C. In addition, it was found that a short 20 bp oligo fully complementary to A could be used to completely block the toehold of file A and essentially “delete” it from the data base. See FIG. 20B, bottom. A file can also be simply extracted from a database to delete it as well. However, the presently disclosed form of “blocking” deletion can provide a way to ensure any leftover file strands that were not completely extracted would not be spuriously accessed in the future.

Conclusions:

FIGS. 14A-14D and 15 show the creation of an overhang/toehold structure for DNA strands. FIGS. 14A-14D and 15 also show how a DNA oligo can be used to bind the toehold and extract specific DNA strands. Different temperatures for this process were tested and it was found that operating at 25° C. was as effective as at 35 or 45° C. (FIG. 15). FIGS. 16A-16C provided additional studies where different temperatures between 15-55° C. and also the effect of different DNA oligo lengths on the efficiency with which oligos were able to bind the toehold and extract specific DNA strands were studied. Again, it was determined that temperature had little effect, but that a toehold and DNA oligo length of 20 bp or higher was preferred for strong file access efficiency.

FIGS. 17A-17C show how in vitro transcription (IVT) can be used to access information from strands by copying it to RNA without destroying the original DNA. The recovery of the DNA was quantified. In FIGS. 18A, 18D, and 19, the length of time used for IVT was studied and it was found that the longer the IVT, the lower the recovery of DNA. Accordingly, an 8 hour process was used in subsequent studies as an optimal IVT time. This optimization of minimizing IVT time shortens the time that the DNA is exposed to a 37° C. temperature step. In addition, it was determined that by extracting the file first, then performing IVT, exposure of the rest of the library to the 37° C. temperature IVT step can be avoided, which can improve library stability/longevity.

FIGS. 20A, 20B, 21A, and 21B show how lock, unlock, rename, and delete file operations can be performed through addition of specially designed DNA oligos that bind the toehold region of target file strands. The locking process appears to be optimized at 98° C., but can function adequately at 45° C. See FIG. 21A. Also, the unlocking process can be performed at 25° C. and is not improved by increasing the temperature to 35-98° C. See FIGS. 20A, 20B, 21A, and 21B. After unlocking, the file access temperature (where the DNA oligo is used to bind toeholds and pull out strands) can be held at 25° C. Increasing that temperature to 35-75° C. does not improve the file access efficiency.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
US2018/0265921A1
CA2878042 A1
WO2014014991 A2
EP2875458 A2
US20050053968 A1
WO2013178801 A2
US20130019572 A1
WO2004088585 A2
WO2013178801 A2
US20150261664 A1
WO2015090879 A1
CA2874540 A1
EP2856375 A2
US20040001371 A1
WO2004088585 A2
U.S. Pat. No. 7,659,175
U.S. patent application Ser. No. 11/766,222
U.S. Pat. No. 9,262,738
WO2014014991

While the systems and methods have been described herein in reference to specific aspects, features, and illustrative embodiments, it will be appreciated that the utility of the subject matter is not thus limited, but rather extends to and encompasses numerous other variations, modifications and alternative embodiments, as will suggest themselves to those of ordinary skill in the field of the present subject matter, based on the disclosure herein. Various combinations and sub-combinations of the structures and features described herein are contemplated and will be apparent to a skilled person having knowledge of this disclosure. Any of the various features and elements as disclosed herein can be combined with one or more other disclosed features and elements unless indicated to the contrary herein. Correspondingly, the subject matter as hereinafter claimed is intended to be broadly construed and interpreted, as including all such variations, modifications and alternative embodiments, within its scope and including equivalents of the claims.

1. A process for extracting a data file from a database, wherein the data file comprises information encoded into one or more polynucleotide strands and wherein the database comprises a plurality of polynucleotide strands, the process comprising:
providing an oligonucleotide primer that selectively binds a polynucleotide strand bearing the data file, wherein the primer is labeled with a chemical moiety;
contacting the database with the primer and with a magnetic bead comprising a corresponding chemical group that binds the primer moiety; and
extracting the one or more polynucleotide strands bearing the data file using a magnet.

2. The process of claim 1, wherein the one or more polynucleotide strands comprise a deoxyribonucleic acid (DNA) strand, optionally wherein the DNA strand can be single stranded (ss) or double stranded (ds).

3. The process of claim 1, wherein the chemical moiety on the primer and the corresponding chemical group are selected from the group consisting of biotin-streptavidin, fluorescein-antibody, digoxigenin-antibody, and polyA-polyT oligomers.

4. The process of claim 1, comprising amplifying the one or more polynucleotide strands bearing the data file prior to extracting the file using the magnet.

5. The process of claim 1, comprising sequencing the one or more polynucleotide strands bearing the data file.

6. The process of claim 5, comprising decoding the data file from sequencing data obtained from sequencing the one or more polynucleotide strands bearing the data file; performing error analysis of sequencing data to infer where errors in the process might be occurring and/or the frequency of the errors; or a combination thereof.

7. The process of claim 1, wherein the data file can be repeatedly extracted from the same database and wherein the process is a nondestructive process.

8. A process for expanding a number of unique data files in a database that can be addressed with a predetermined number of oligonucleotide primers, wherein the unique data files each comprise information encoded into one or more polynucleotide strands and wherein the database comprises a plurality of polynucleotide strands, the process comprising designing two or more primers within the predetermined number of oligonucleotide primers that each selectively bind the one or more polynucleotide strands; and assigning a hierarchy to the two or more oligonucleotide primers within the predetermined number of oligonucleotide primers.

9. The process of claim 8, wherein the one or more polynucleotide strands comprise a deoxyribonucleic acid (DNA) strand, optionally wherein the DNA strand can be single stranded (ss) or double stranded (ds).

10. The process of claim 8, wherein assigning a hierarchy to primers comprises nesting two or more primer binding sites adjacent to a data file to be amplified using an oligonucleotide primer complementary to one of the two or more primer binding sites.

11. The process of claim 8, comprising amplifying a data file using a primer for which a hierarchy has been assigned, optionally wherein the primer binds to one of the two or more primer binding sites.

12. The process of claim 8, wherein the primer is labeled with a chemical moiety and the process further comprises contacting the database with the primer and with a magnetic bead comprising a corresponding chemical group that binds the primer moiety; and extracting the one or more polynucleotide strands bearing the data file using a magnet.

13. The process of claim 12, wherein the chemical moiety on the primer and the corresponding chemical group are selected from the group consisting of biotin-streptavidin, fluorescein-antibody, digoxigenin-antibody, and polyA-polyT oligomers.

14. The process of claim 12, comprising amplifying the polynucleotide strand bearing the data file strand prior to extracting the file using the magnet.

15. The process of claim 8, comprising sequencing the one or more polynucleotide strands bearing the data file.

16. The process of claim 15, comprising decoding the data file from sequencing data obtained from sequencing the one or more polynucleotide strands bearing the data file; performing error analysis of sequencing data to infer where errors in the process might be occurring and/or the frequency of the errors; or a combination thereof.

17. The process of claim 8, wherein the data file can be repeatedly extracted from the same database and wherein the process is a nondestructive process.

18. A process for differentially reading information encoded into one or more polynucleotide strands, the process comprising:
providing a database comprising a plurality of the polynucleotide strands;
providing an oligonucleotide primer that selectively binds one or more polynucleotide strands bearing information;
contacting the database with the primer under conditions where the selective binding of the primer to the polynucleotide strand bearing information is controlled; and
differentially reading information encoded into the polynucleotide strand based on the binding conditions.

19. The process of claim 18, wherein the polynucleotide strand comprises a deoxyribonucleic acid (DNA) strand, optionally wherein the DNA strand can be single stranded (ss) and/or double stranded (ds).

20. The process of claim 18, wherein the conditions where the selective binding of the primer to the polynucleotide strand bearing information is controlled comprise conditions wherein one or more mis-match interactions between the primer and the polynucleotide strand bearing information occur.

21. The process of claim 18, wherein the conditions where the selective binding of the primer to the file is controlled comprise lowering a temperature under which the binding is allowed to proceed, increasing a concentration of primer, varying a binding buffer composition, length of primer, and combinations thereof.

22. The process of claim 18, comprising amplifying the polynucleotide strand bearing information under the conditions where the selective binding of the primer to the file is controlled.

23. A process for extracting a data file from a database, wherein the data file comprises information encoded into a polynucleotide strand, the process comprising:
providing a database comprising a plurality of polynucleotide strands, wherein the data file comprises information encoded into one or more double stranded (ds) polynucleotide strands;
providing a physical occlusion that provides selective access to the data file;
contacting the database with a reagent that selectively binds a location on one or more of the polynucleotide strands not occluded by physical occlusion; and
extracting the one or more polynucleotide strands bearing the data file using the reagent.

24. The process of claim 23, wherein the database comprises a plurality of DNA strands, wherein the DNA strands comprise doubled-stranded DNA (dsDNA).

25. The process of claim 23, wherein the physical occlusion comprises a single strand polynucleotide overhang (ss overhang) on the one or more doubled-stranded (ds) polynucleotide strands bearing the data file; and
wherein the process comprises:
providing an oligonucleotide primer that selectively binds the ss overhang, wherein the primer is labeled with a chemical moiety;
contacting the database with the primer and with a magnetic bead comprising a corresponding chemical group that binds the primer moiety; and
extracting the one or more polynucleotide strands bearing the data file using a magnet.

26. The process of claim 25, wherein the chemical moiety on the primer and the corresponding chemical group are selected from the group consisting of biotin-streptavidin, fluorescein-antibody, digoxigenin-antibody, and polyA-polyT oligomers.

27. The process of claim 25, wherein the ss overhang is hidden by a ss sequence comprising a sequence that is complementary to the ss overhang and a toehold switch sequence, whereby the data file is hidden from extraction; and extracting the one or more polynucleotide strands bearing the data file further comprises contacting the database with a key nucleic acid strand comprising the ss overhang and a sequence complementary to the toehold sequence, whereby the ss overhang on the ds polynucleotide strand is revealed and the labeled primer that selectively binds the ss overhang can bind the ss overhang.

28. The process of claim 25, wherein the polynucleotide strand comprises a RNA polymerase promoter sequence, optionally a T7 promoter sequence, and extracting the polynucleotide strand bearing the data file comprises extracting the ss overhang strand using the labeled primer, adding a RNA polymerase to transcribe the polynucleotide strand bearing the data file, and creating ribonucleic acid (RNA), wherein information in the data file can be derived from the RNA.

29. The process of claim 28, wherein the extracted polynucleotide strand bearing the data file can be returned to the original database while the RNA is used to derive the data file.

30. The process of claim 23, wherein the physical occlusion comprises a DNA binding molecule, such as but not limited to an archaeal histone proteins, a poly-cationic polymer, a dendrimer, and/or a nucleosome; and wherein the process comprises:
providing a reagent that binds a sequence not occluded by the DNA binding molecule, wherein the reagent is labeled with a chemical moiety and wherein the sequence not occluded by the DNA binding molecule is associated with the data file;
contacting the database with the reagent and with a magnetic bead comprising a corresponding chemical group that binds the chemical moiety; and
extracting the one or more polynucleotide strands bearing the data file using a magnet.

31. The process of claim 30, wherein the reagent comprises an oligonucleotide and/or a binding protein, optionally wherein the oligonucleotide comprises a guide RNA and the binding protein comprises dCas9, optionally wherein the oligonucleotide is labeled with a chemical moiety, or optionally wherein the binding protein is a TALE and/or a ZF.

32. The process of claim 23, comprising sequencing the one or more polynucleotide strands bearing the data file.

33. The process of claim 32, comprising decoding the data file from sequencing data obtained from sequencing the one or more polynucleotide strands bearing the data file; performing error analysis of sequencing data to infer where errors in the process might be occurring and/or the frequency of the errors; or a combination thereof.

34. The process of claim 23, wherein the data file can be repeatedly extracted from the same database and wherein the process is a nondestructive process.

35. A system suitable for use in carrying out a process as set forth in claim 1.
